SEQUENCE LISTING (132 sequences)

SEQ ID NO: 1 - protein, 122 aa, synthetic construct [sequence omitted]

SEQ ID NO: 2 - protein, 107 aa, synthetic construct
DIQMTQSPSSLSASVGDRVTITCHASQNINVWLSWYQQKPGKAPKLLIYKASNLHTGVPSRFTGSGSGTDFTFTISSLQPEDIATYYCQQGQTYPYTFGQGTKLEIK

SEQ ID NO: 3 - protein, 107 aa, synthetic construct
DIQMTQSPSSLSASVGDRVTITCHASQNINVWLSWYQQKPGKAPKLLIYKASNLHTGVPSRFSGSGSGTDFTFTISSLQPEDIATYYCQQGQTYPYTFGQGTKLEIK

SEQ ID NO: 4 - protein, 451 aa, synthetic construct
QVQLVQSGAEVKKPGASVKLSCKASGYIFTRYYMYWVKQRPGQGLEWIGEINPNNGGTKFNEKFKNRATLTVDKSISTAYMELSRLRSDDTAVYYCSREDDYDGTPFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 448 aa, synthetic construct
QVQLVQSGAEVKKPGASVKLSCKASGYIFTRYYMYWVKQRPGQGLEWIGEINPNNGGTKFNEKFKNRATLTVDKSISTAYMELSRLRSDDTAVYYCSREDDYDGTPFAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 6 - protein, 214 aa, synthetic construct
DIQMTQSPSSLSASVGDRVTITCHASQNINVWLSWYQQKPGKAPKLLIYKASNLHTGVPSRFTGSGSGTDFTFTISSLQPEDIATYYCQQGQTYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 214 aa, synthetic construct
DIQMTQSPSSLSASVGDRVTITCHASQNINVWLSWYQQKPGKAPKLLIYKASNLHTGVPSRFSGSGSGTDFTFTISSLQPEDIATYYCQQGQTYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - DNA, 1356 bp, synthetic construct
  source 1..1356: mol_type = other DNA
caggtccaactggtgcagtctggggctgaagtgaagaagcctggggcttcagtgaagttgtcctgcaaggcttctggctacatcttcaccagatactatatgtattgggtgaagcagaggcctggacaaggccttgagtggattggagagattaatcctaacaatggtggtactaagttcaatgagaagttcaagaacagggccacactgactgtagacaaatccatcagcacagcatacatggagctcagcaggctgagatctgacgacacggcggtctattattgttcaagagaggatgattacgacgggaccccctttgctatggactactggggtcaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaattcgaggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 9 - DNA, 1347 bp, synthetic construct
  source 1..1347: mol_type = other DNA
caggtccaactggtgcagtctggggctgaagtgaagaagcctggggcttcagtgaagttgtcctgcaaggcttctggctacatcttcaccagatactatatgtattgggtgaagcagaggcctggacaaggccttgagtggattggagagattaatcctaacaatggtggtactaagttcaatgagaagttcaagaacagggccacactgactgtagacaaatccatcagcacagcatacatggagctcagcaggctgagatctgacgacacggcggtctattattgttcaagagaggatgattacgacgggaccccctttgctatggactactggggtcaaggaaccctggtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaatgtagatcacaagcccagcaacaccaaggtggacaagagagttgagtccaaatatggtcccccatgcccaccatgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggttga

SEQ ID NO: 10 - DNA, 645 bp, synthetic construct
  source 1..645: mol_type = other DNA
gacatccagatgacccagtctccatccagtctgtctgcatccgtaggagacagagtcaccatcacttgccatgccagtcagaacattaatgtttggttatcttggtaccagcagaaaccaggaaaagcccctaaactcttgatctataaggcttccaacttgcacacaggcgtcccatcaagatttactggcagtggatctggaacagatttcacattcaccatcagcagcctgcagcctgaagacattgccacttactactgtcaacagggtcaaacttatccatacacgttcggacaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 11 - DNA, 645 bp, synthetic construct
  source 1..645: mol_type = other DNA
gacatccagatgacccagtctccatccagtctgtctgcatccgtaggagacagagtcaccatcacttgccatgccagtcagaacattaatgtttggttatcttggtaccagcagaaaccaggaaaagcccctaaactcttgatctataaggcttccaacttgcacacaggcgtcccatcaagatttagtggcagtggatctggaacagatttcacattcaccatcagcagcctgcagcctgaagacattgccacttactactgtcaacagggtcaaacttatccatacacgttcggacaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 12 - protein, 5 aa, synthetic construct
RYYMY

SEQ ID NO: 13 - protein, 17 aa, synthetic construct
EINPNNGGTKFNEKFKN

SEQ ID NO: 14 - protein, 13 aa, synthetic construct
EDDYDGTPFAMDY

SEQ ID NO: 15 - protein, 11 aa, synthetic construct
HASQNINVWLS

SEQ ID NO: 16 - protein, 7 aa, synthetic construct
KASNLHT

SEQ ID NO: 17 - protein, 9 aa, synthetic construct
QQGQTYPYT

SEQ ID NO: 18 - protein, 5 aa, synthetic construct
RYYLY

SEQ ID NO: 19 - protein, 17 aa, synthetic construct
EINPNNGGTKFNEKFKS

SEQ ID NO: 20 - protein, 13 aa, synthetic construct
EDDYDGTPDAMDY

SEQ ID NO: 21 - protein, 11 aa, synthetic construct
HASQNINLWLS

SEQ ID NO: 22 - protein, 7 aa, synthetic construct
RASNLHT

SEQ ID NO: 23 - protein, 9 aa, synthetic construct
QQGHSYPYT

SEQ ID NO: 24 - protein, 513 aa, Homo sapiens
MKMASFLAFLLLNFRVCLLLLQLLMPHSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGTLPVLLLLLGGAGYFLWQQQEEKKTQFRKKKREQELREMAWSTMKQEQSTRVKLLEELRWRSIQYASRGERHSAYNEWKKALFKPADVILDPKTANPILLVSEDQRSVQRAKEPQDLPDNPERFNWHYCVLGCESFISGRHYWEVEVGDRKEWHIGVCSKNVQRKGWVKMTPENGFWTMGLTDGNKYRTLTEPRTNLKLPKPPKKVGVFLDYETGDISFYNAVDGSHIHTFLDVSFSEALYPVFRILTLEPTALTICPA

SEQ ID NO: 25 - protein, 334 aa, Homo sapiens
MKMASSLAFLLLNFHVSLLLVQLLTPCSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSNLHVEVKGYEDGGIHLECRSTGWYPQPQIQWSNAKGENIPAVEAPVVADGVGLYEVAASVIMRGGSGEGVSCIIRNSLLGLEKTASISIADPFFRSAQPWIAALAGTLPILLLLLAGASYFLWRQQKEITALSSEIESEQEMKEMGYAATEREISLRESLQEELKRKKIQYLTRGEESSSDTNKSA

SEQ ID NO: 26 - protein, 584 aa, Homo sapiens
MKMASSLAFLLLNFHVSLFLVQLLTPCSAQFSVLGPSGPILAMVGEDADLPCHLFPTMSAETMELRWVSSSLRQVVNVYADGKEVEDRQSAPYRGRTSILRDGITAGKAALRIHNVTASDSGKYLCYFQDGDFYEKALVELKVAALGSDLHIEVKGYEDGGIHLECRSTGWYPQPQIKWSDTKGENIPAVEAPVVADGVGLYAVAASVIMRGSSGGGVSCIIRNSLLGLEKTASISIADPFFRSAQPWIAALAGTLPISLLLLAGASYFLWRQQKEKIALSRETEREREMKEMGYAATEQEISLREKLQEELKWRKIQYMARGEKSLAYHEWKMALFKPADVILDPDTANAILLVSEDQRSVQRAEEPRDLPDNPERFEWRYCVLGCENFTSGRHYWEVEVGDRKEWHIGVCSKNVERKKGWVKMTPENGYWTMGLTDGNKYRALTEPRTNLKLPEPPRKVGIFLDYETGEISFYNATDGSHIYTFPHASFSEPLYPVFRILTLEPTALTICPIPKEVESSPDPDLVPDHSLETPLTPGLANESGEPQAEVTSLLLPAHPGAEVSPSATTNQNHKLQARTEALY

SEQ ID NO: 27 - protein, 237 aa, Macaca fascicularis
MGSSLAFLLLSFHVCVLLLQLLMPHSAQFAVVGPPGPILAMVGEDADLPCHLFPTMSAETMELRWVSSNLRQVVNVYADGKEVEDRQSAAYRGRTSILRDGITAGKAALRIHNVTASDSGKYLCYFQDGDFYEKALVELKVAALGSDLHIDVKGYEDGGIHLECRSTGWYPQPQIRWSNDKGENIPAVEAPVFVDGVGLYAVAASVILRGSSGEGVSCTIRSSLLGLEKTTSISIAG

SEQ ID NO: 28 - protein, 237 aa, Macaca fascicularis
MGSSLAFLLLNFHVSFFLVQLLTPCSAQFSVLGPSGPILAMVGEDADLPCHLFPTMSAETMELRWVSSSLRQVVNVYADGKEVEDRQSAPYRGRTSILRDDIAAGKAALRIHNVTASDSGKYLCYFQDADFYEKALVELKVAALGSNLHVEVKGYEDGGIHLECRSTGWYPQPKIQWSNAKGQNIPAVEAPVVADGVGLYAVAASVIMRGGSGESVSCIIRNSVLGLEKTASISIAD

SEQ ID NO: 29 - protein, 237 aa, Macaca fascicularis
MANFLAFLLLNFRVCLLLVQLLTPCSAQFAVLGPHGPILAMVGEDVDLPCHLFPTMSAETMELRWVSSSLRQVVNVYSDGKEVEDRQSAPYRGRTSILRDGITAGKAALRIHNVTASDSGKYLCYFQDGDFYEKALVELKVAALGSDLHIEVKGYEDGGIHLECRSTGWYPQPQIQWSNTKGQHIPAVKAPVVADGVGLYAVAASVIMRGSSGEGVSCIIRNSLLGLEKTASISITD

SEQ ID NO: 30 - protein, 654 aa, synthetic construct
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGDDDDKGSGSGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 447 aa, synthetic construct
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 214 aa, synthetic construct
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 113 aa, synthetic construct [sequence omitted]

SEQ ID NO: 36 - protein, 107 aa, synthetic construct [sequence omitted]

SEQ ID NO: 37 - protein, 120 aa, synthetic construct
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSS

SEQ ID NO: 38 - protein, 107 aa, synthetic construct
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIK

SEQ ID NO: 39 - protein, 5 aa, synthetic construct
NSGMH

SEQ ID NO: 40 - protein, 17 aa, synthetic construct
VIWYDGSKRYYADSVKG

SEQ ID NO: 41 - protein, 4 aa, synthetic construct
NDDY

SEQ ID NO: 42 - protein, 11 aa, synthetic construct
RASQSVSSYLA

SEQ ID NO: 43 - protein, 7 aa, synthetic construct
DASNRAT

SEQ ID NO: 44 - protein, 9 aa, synthetic construct
QQSSNWPRT

SEQ ID NO: 45 - protein, 8 aa, synthetic construct
GGSFSDYY

SEQ ID NO: 46 - protein, 16 aa, synthetic construct
EINHRGSTNSNPSLKS

SEQ ID NO: 47 - protein, 12 aa, synthetic construct
GYSDYEYNWFDP

SEQ ID NO: 48 - protein, 11 aa, synthetic construct
RASQSISSYLA

SEQ ID NO: 49 - protein, 9 aa, synthetic construct
QQRSNWPLT

SEQ ID NO: 50 - protein, 446 aa, synthetic construct
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 51 - protein, 218 aa, synthetic construct
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 119 aa, synthetic construct
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSS

SEQ ID NO: 53 - protein, 111 aa, synthetic construct
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIK

SEQ ID NO: 54 - protein, 5 aa, synthetic construct
DYNVD

SEQ ID NO: 55 - protein, 17 aa, synthetic construct
DINPNDGGTIYAQKFQE

SEQ ID NO: 56 - protein, 10 aa, synthetic construct
NYRWFGAMDH

SEQ ID NO: 57 - protein, 15 aa, synthetic construct
KASQSLDYEGDSDMN

SEQ ID NO: 58 - protein, 7 aa, synthetic construct
GASNLES

SEQ ID NO: 59 - protein, 9 aa, synthetic construct
QQSTEDPRT

SEQ ID NO: 60 - protein, 271 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVQSGAEVKKPGASVKVSCKASGYTFTDYNMDWVRQAPGQGLEWMGDINPDNGVTIYNQKFEGRVTMTTDTSTSTAYMELRSLRSDDTAVYYCAREADYFYFDYWGQGTTLTVSSGGCGGGKVAACKEKVAALKEKVAALKEKVAALKE

SEQ ID NO: 61 - protein, 496 aa, synthetic construct
DIQMTQSPSSLSASVGDRVTITCRASQDVSSVVAWYQQKPGKAPKLLIYSASYRYTGVPSRFSGSGSGTDFTLTISSLQPEDFATYYCQQHYSTPWTFGGGTKLEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGEVAACEKEVAALEKEVAALEKEVAALEKESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 62 - protein, 118 aa, synthetic construct
QVQLVQSGAEVKKPGASVKVSCKASGYTFTDYNMDWVRQAPGQGLEWMGDINPDNGVTIYNQKFEGRVTMTTDTSTSTAYMELRSLRSDDTAVYYCAREADYFYFDYWGQGTTLTVSS

SEQ ID NO: 63 - protein, 111 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIK

SEQ ID NO: 64 - protein, 107 aa, synthetic construct
DIQMTQSPSSLSASVGDRVTITCRASQDVSSVVAWYQQKPGKAPKLLIYSASYRYTGVPSRFSGSGSGTDFTLTISSLQPEDFATYYCQQHYSTPWTFGGGTKLEIK

SEQ ID NO: 65 - protein, 5 aa, synthetic construct
DYNMD

SEQ ID NO: 66 - protein, 17 aa, synthetic construct
DINPDNGVTIYNQKFEG

SEQ ID NO: 67 - protein, 9 aa, synthetic construct
EADYFYFDY

SEQ ID NO: 68 - protein, 15 aa, synthetic construct
RASESVDNYGMSFMN

SEQ ID NO: 69 - protein, 7 aa, synthetic construct
AASNQGS

SEQ ID NO: 70 - protein, 9 aa, synthetic construct
QQSKEVPYT

SEQ ID NO: 71 - protein, 5 aa, synthetic construct
SYWMN

SEQ ID NO: 72 - protein, 17 aa, synthetic construct
VIHPSDSETWLDQKFKD

SEQ ID NO: 73 - protein, 10 aa, synthetic construct
EHYGTSPFAY

SEQ ID NO: 74 - protein, 11 aa, synthetic construct
RASQDVSSVVA

SEQ ID NO: 75 - protein, 7 aa, synthetic construct
SASYRYT

SEQ ID NO: 76 - protein, 7 aa, synthetic construct
HYSTPWT

SEQ ID NO: 77 - protein, 444 aa, synthetic construct
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 78 - protein, 218 aa, synthetic construct
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 118 aa, synthetic construct
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSS

SEQ ID NO: 80 - protein, 111 aa, synthetic construct
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIK

SEQ ID NO: 81 - protein, 5 aa, synthetic construct
SYNMH

SEQ ID NO: 82 - protein, 17 aa, synthetic construct
DIYPGNGDTSYNQKFKG

SEQ ID NO: 83 - protein, 9 aa, synthetic construct
VGGAFPMDY

SEQ ID NO: 84 - protein, 15 aa, synthetic construct
RASESVEYYGTSLMQ

SEQ ID NO: 85 - protein, 7 aa, synthetic construct
AASNVES

SEQ ID NO: 86 - protein, 9 aa, synthetic construct
QQSRKDPST

SEQ ID NO: 87 - protein, 443 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 227 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 449 aa, synthetic construct
EVQLVESGGGLVQPGGSLRLSCAASGFNIKTTYMHWVRQAPGKGLEWVGRIDPADDNTKYAPKFQGKATISADTSKNTAYLQMNSLRAEDTAVYYCVRDFGYVAWFAYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 90 - protein, 214 aa, synthetic construct
DIVMTQSPLSLPVTPGEPASISCRASQSVDNYVAWYLQKPGQSPQLLIYYASNRYIGVPDRFSGSGSGTDFTLKISRVEAEDVGVYYCQQHYSSPYTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 113 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKSS

SEQ ID NO: 92 - protein, 120 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSS

SEQ ID NO: 93 - protein, 119 aa, synthetic construct
EVQLVESGGGLVQPGGSLRLSCAASGFNIKTTYMHWVRQAPGKGLEWVGRIDPADDNTKYAPKFQGKATISADTSKNTAYLQMNSLRAEDTAVYYCVRDFGYVAWFAYWGQGTLVTVSS

SEQ ID NO: 94 - protein, 107 aa, synthetic construct
DIVMTQSPLSLPVTPGEPASISCRASQSVDNYVAWYLQKPGQSPQLLIYYASNRYIGVPDRFSGSGSGTDFTLKISRVEAEDVGVYYCQQHYSSPYTFGQGTKVEIK

SEQ ID NO: 95 - protein, 456 aa, synthetic construct
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 220 aa, synthetic construct
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 126 aa, synthetic construct
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSS

SEQ ID NO: 98 - protein, 113 aa, synthetic construct
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIK

SEQ ID NO: 99 - protein, 10 aa, synthetic construct
GDSVSSNSAA

SEQ ID NO: 100 - protein, 9 aa, synthetic construct
TYYRFKWYS

SEQ ID NO: 101 - protein, 16 aa, synthetic construct
TRESTTYDLLAGPFDY

SEQ ID NO: 102 - protein, 12 aa, synthetic construct
QTVLYSSNNKKY

SEQ ID NO: 103 - skipped

SEQ ID NO: 104 - protein, 9 aa, synthetic construct
QQYYSTPFT

SEQ ID NO: 105 - protein, 451 aa, synthetic construct
QVQLVESGGGVVQPGRSLRLSCAASGFTFSSYAMHWVRQAPGKGLEWVAVISYAGEVKYYADSVKGRFTISRDNSKNTLYLQMNSLRAEDTAVYYCARDPLPLHYYGMDVWGQGTTVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 220 aa, synthetic construct; identical to SEQ ID NO: 96

SEQ ID NO: 107 - protein, 452 aa, synthetic construct
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 108 - protein, 219 aa, synthetic construct
QIVLTQSPATLSLSPGERATLSCSASSKHTNLYWSRHMYWYQQKPGQAPRLLIYLTSNRATGIPARFSGSGSGTDFTLTISSLEPEDFAVYYCQQWSSNPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 121 aa, synthetic construct
QVQLVESGGGVVQPGRSLRLSCAASGFTFSSYAMHWVRQAPGKGLEWVAVISYAGEVKYYADSVKGRFTISRDNSKNTLYLQMNSLRAEDTAVYYCARDPLPLHYYGMDVWGQGTTVTVSS

SEQ ID NO: 110 - protein, 110 aa, synthetic construct
QSVLTQPPSASGTPGQRVTISCSGSSSNIGRRPVNWYQQLPGTAPKLLIYSQNQRPSGVPDRFSGSQSGTSASLAISGLQSEDEADYFCAVWDDIGRVLQLGGGTQLTVL

SEQ ID NO: 111 - protein, 122 aa, synthetic construct
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSS

SEQ ID NO: 112 - protein, 112 aa, synthetic construct
QIVLTQSPATLSLSPGERATLSCSASSKHTNLYWSRHMYWYQQKPGQAPRLLIYLTSNRATGIPARFSGSGSGTDFTLTISSLEPEDFAVYYCQQWSSNPFTFGQGTKLEIK

SEQ ID NO: 113 - protein, 8 aa, synthetic construct
GFTFSSYA

SEQ ID NO: 114 - protein, 8 aa, synthetic construct
ISYAGEVK

SEQ ID NO: 115 - protein, 14 aa, synthetic construct
ARDPLPLHYYGMDV

SEQ ID NO: 116 - protein, 8 aa, synthetic construct
SSNIGRRP

SEQ ID NO: 117 - skipped

SEQ ID NO: 118 - protein, 11 aa, synthetic construct
AVWDDIGRVLQ

SEQ ID NO: 119 - protein, 8 aa, synthetic construct
GFTFSDYG

SEQ ID NO: 120 - protein, 8 aa, synthetic construct
ISSGSYTI

SEQ ID NO: 121 - protein, 15 aa, synthetic construct
ARRAPNSFYEYYFDY

SEQ ID NO: 122 - protein, 11 aa, synthetic construct
SKHTNLYWSRH

SEQ ID NO: 123 - skipped

SEQ ID NO: 124 - protein, 9 aa, synthetic construct
QQWSSNPFT

SEQ ID NO: 125 - protein, 122 aa, synthetic construct
QVQLVQSGAEVKKPGASVKVSCKASGYTFTRYYLYWVKQRPGQGLEWIGEINPNNGGTKFNEKFKSRATMTVDKSTRTTYMELSSLRSEDTAVYYCSREDDYDGTPDAMDYWGQGTLVTVSS

SEQ ID NO: 126 - protein, 107 aa, synthetic construct
DIQMTQSPSSLSASVGDRVTITCHASQNINLWLSWYQQKPGKAPKLLIYRASNLHTGVPSRFSGSGSATDFTFTISSLQPEDIATYYCQQGHSYPYTFGGGTKVDIK

SEQ ID NO: 127 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 128 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 129 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 130 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 131 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 132 - protein, 119 aa, synthetic construct
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSS

(19) United States
(12) Patent Application Publication
(10) Pub. No.: US 2026/0242479 A1
(43) Pub. Date: Aug. 20, 2026

(54) COMBINATION OF BTN3A ACTIVATING ANTIBODY AND IMMUNE CHECKPOINT INHIBITORS

(71) Applicant: Imcheck Therapeutics, Marseille (FR)

(72) Inventors: Aude DE GASSART, Marseille (FR); Kieu-Suong LE, Marseille (FR)

(21) Appl. No.: 19/158,538

(22) PCT Filed: Feb. 22, 2024
(86) PCT No.: PCT/EP2024/054490

§ 371 Date: Aug. 21, 2025

(30) Foreign Application Priority Data
Feb. 23, 2023 (EP) 23305243.0

(51) Int. Cl. C07K 16/28; A61K 38/17; A61K 39/00; A61K 39/395; A61P 35/00

(52) U.S. Cl. CPC C07K 16/2827; A61K 38/1774; A61K 39/39558; A61P 35/00; C07K 16/2803; C07K 16/2818; A61K 2039/507; A61K 2039/54; A61K 2039/545; C07K 2317/92

(57) ABSTRACT

The present application relates to a therapeutic combination of an BTN3A activating antibody and immune checkpoint inhibitors selected from LAG-3 inhibitors (anti-LAG-3 antibody and LAG-3 fusion protein), anti-TIGIT antibody or anti-TIM-3 antibody, that is particularly useful for the treatment of cancer. The present disclosure more particularly related to the combined use of a BTN3A activating antibody that specifically binds to BTN3A and activates the cytolytic function of Vγ9Vδ2 T cells and LAG-3 inhibitors which can directly bind LAG-3 molecules or their ligands, blocking the interaction between LAG-3 and their ligands, in order to promote a cooperative anti-tumor effect.

It is hereafter disclosed a therapeutic combination of a BTN3A activating antibody and immune checkpoint inhibitors selected from LAG-3 inhibitors (e.g. anti-LAG-3 antibody or LAG-3 fusion protein), anti-TIGIT antibody, or anti-TIM-3 antibody, that is particularly useful for the treatment of cancer. The present disclosure is more particularly related to the combined use of a BTN3A activating antibody that specifically binds to BTN3A and activates the cytolytic function of Vγ9Vδ2 T cells and LAG-3 inhibitors which can directly bind LAG-3 molecules or their ligands and block the interaction between LAG-3 and their ligands, in order to promote a cooperative anti-tumor effect.

### BACKGROUND

Tumors evade immune-mediated recognition through several mechanisms. Among them, immune checkpoint inhibitory pathways have been well studied (Thommen et al., 2018 Cancer Cell. April 9; 33(4):547-562), in particular the immune checkpoint receptors PD-1 and CTLA-4 that are strongly expressed on T cells rendering them unresponsive or exhausted. Immune checkpoint inhibitor therapy targeting these receptors has shown promising clinical responses across a broad range of cancers. However, many patients still do not respond to them and there is a need to identify alternative targets and approaches to improve therapeutic benefit for patients (Ribas and Wolchok 2018, Science. March 23; 359(6382):1350-1355; Postow et al 2015. J Clin Oncol. 2015 Jun. 10; 33(17):1974-82; Kong X. 2020. Adv Exp Med Biol. 1248:61-82). Beyond PD-1/PD-L1 and CTLA-4, novel immune checkpoint receptors such as LAG-3, TIM-3 and TIGIT have been well studied in cancer immunity and antibody-based therapies targeting these inhibitory receptors have been developed and investigated in clinical trials as monotherapy or in combination with other agents (Chauvin et al. 2020. J Immunother Cancer. September; 8(2):e000957; He et al 2018. Oct. 16; 11:7005-7009; Huo et al. 2022. Front Immunol. July 26; 13:956090). In 2022, Opdualag, the combination of Relatlimab, an anti-LAG-3 mAb developed by Bristol Myers Squibb (BMS), and nivolumab, an anti-PD-1 monoclonal antibody, was approved by the FDA as a single formulation for treatment of unresectable or metastatic melanoma showing an important therapeutic benefit compared to nivolumab alone (Chocarro et al 2022. Cutting-Edge: Preclinical and Clinical Development of the First Approved Lag-3 Inhibitor. Cells. July 30; 11(15):2351). This indicates that combination of several immune checkpoint inhibitors may be of therapeutic benefit for patients. Furthermore, besides MHC-restricted αβ T cells that have been largely documented during immune checkpoint inhibitor therapy, the role of immune checkpoint receptors on unconventional non-MHC-restricted T cells is not well characterized, especially given the fact that immune checkpoint receptors are also expressed by these cells.

Although these drugs represent major advances in cancer therapy, there is still an unmet medical need for a large proportion of cancer patient populations that do not respond to the currently available treatments.

Among attractive unconventional T cells, Vγ9Vδ2 T cells are emerging as promising effector cells that harbor strong cytolytic and pro-inflammatory activities and are associated with good prognosis in cancer patients (Holtmeier et al 2005. Chem Immunol Allergy. 86:151-183; Gentles et al 2015. Nat Med. August; 21(8):938-945; Tosolini et al. 2017. Oncoimmunology. February 6; 6(3):e1284723).

A number of Vγ9Vδ2 T cell-based immuno-oncology therapeutic approaches have thus been explored in a variety of tumors in recent years using either in vivo activation of Vγ9Vδ2 T cells with aminobisphosphonates (ABP) such as Zoledronate or synthetic pAg (i.e. BrHPP) in combination with IL-2, or adoptive transfer of autologous or allogeneic Vγ9Vδ2 T cells to patients following in vitro/ex vivo expansion (Kabelitz et al 2020. Cell Mol Immunol. September; 17(9):925-939). While these Vγ9Vδ2 T cell-based therapies appear to be safe, clinical responses obtained were variable among patients (Kabelitz et al 2020. Cell Mol Immunol. September; 17(9):925-939), suggesting the need to better understand the underlying mechanisms regulating Vγ9Vδ2 T responses in the tumor microenvironment to develop more effective immunotherapies. In tumor immunosurveillance, Vγ9Vδ2 T cell antitumoral activity is triggered by TCR-mediated recognition of pAg which is molecularly dependent on transmembrane butyrophilin (BTN) molecules BTN3A1 and BTN2A1 (Kabelitz et al 2020. Cell Mol Immunol. September; 17(9):925-939).

BTN3A activating antibodies have been described as potent activator of Vγ9Vδ2 T cells (Harly et al 2012. Blood, September 13; 120(11):2269-79; De Gassart et al 2021. Sci Transl Med, 2021 Oct. 20; 13(616):eabj0835). Among them, ICT01 is a first-in-class anti-BTN3A mAb activating Vγ9Vδ2 T cells and being evaluated in a Phase 1/2a clinical study in solid tumors and hematologic malignancies as single agent and in combination with PD-1 blockade (Pembrolizumab) (EVICTION, NCT04243499).

The present disclosure relies on the use of BTN3A activating antibodies, such as ICT01, in combination with LAG-3 or TIM-3 or TIGIT blockade, and optionally in combination with PD-1 inhibitors, to synergistically enhance BTN3A-mediated tumor killing and/or activation of Vγ9Vδ2 T cells and more globally enhance anti-tumor immunity mediated by immune effector cells.

### SUMMARY

The present disclosure relates to a BTN3A activating antibody, for use in the treatment of cancer in a subject in need thereof, wherein a therapeutically effective amount of said BTN3A activating antibody is administered to said subject, in combination with a therapeutically effective amount of an immune checkpoint inhibitor, wherein said immune checkpoint inhibitor is selected from the group consisting of LAG-3 inhibitors, TIGIT inhibitors and TIM-3 inhibitors, and, optionally further in combination with PD-1 inhibitors.

In preferred embodiments, said BTN3A activating antibody is ICT01.

In specific embodiments, said immune checkpoint inhibitor is a LAG-3 inhibitor. In more specific embodiments, said LAG-3 inhibitors are (i) LAG-3 Fc fusion protein such as eftilagimod alpha, (ii) anti-LAG-3 antibody molecule, such as favezelimab, (iii) anti-LAG-3/anti-PD-1 bispecific antibody such as tebotelimab, or (iv) fixed dose combination of anti-LAG-3 antibody and anti-PD-1 antibody such as opdualag (a combination of relatlimab and nivolumab).

In specific embodiments, said immune checkpoint inhibitor is a TIM-3 inhibitor. In more specific embodiments, said TIM-3 inhibitors are (i) anti-TIM-3 antibody molecule, such as sabatolimab, or (ii) anti-LAG-3/anti-PD-1 bispecific antibody such as RO7121661.

In specific embodiments, said immune checkpoint inhibitor is a TIGIT inhibitor. In more specific embodiments, said TIGIT inhibitors are (i) anti-TIGIT antibody molecule, such as tiragolumab, or (ii) anti-TIGIT/anti-PD-1 bispecific antibody such as AZD2936.

### LEGENDS OF THE FIGURES

FIG. 1: Expression of LAG-3, TIM-3 and TIGIT on circulating Vγ9Vδ2 T cells determined by Flow cytometry
A. LAG-3, TIM-3 and TIGIT surface expression on circulating Vγ9Vδ2 T cells in ICT01-stimulated Healthy Donor (HD) hu-PBMC in vitro
B. LAG-3, TIM-3 and TIGIT surface expression on circulating Vγ9Vδ2 T cells in solid tumor patients treated with ICT01 plus Pembrolizumab

Two-way ANOVA and Holm-Sidak's multiple comparisons tests were used: *(p<0.05), **(p<0.01) ***(p<0.001) and ****(p<0.0001)

FIG. 2: Expression of PD-1, LAG-3, TIM-3 and TIGIT on tumor infiltrating Vγ9Vδ2 T cells and CD8+ T cells determined by Flow cytometry
A. Representative flow cytometry profiles for PD-1, LAG-3, TIM-3 and TIGIT surface expression on tumor infiltrating Vγ9Vδ2 T cells and CD8+ T cells from dissociated tumor cells from bladder cancer origin.
B. Percentage of cells positive for PD-1, LAG-3, TIM-3 and TIGIT on tumor-infiltrating Vγ9Vδ2 T cells (left panel) and on tumor-infiltrating CD8+ T cells (right panel) from different solid cancers (bladder cancer (n=15-16), head and neck cancer (n=6-9) and melanoma (n=4)).

FIG. 3: Density of intratumoral CD8 T cells coexpressing PD-1, TIM-3 and LAG-3 assessed by multiplex IHC on Formalin fixed and paraffin-embedded (FFPE) biopsies collected on EVICTION trial patients.

Number of intratumoral CD3+CD8+PD1+LAG3+TIM3+ cells per mm2 of tissue, quantified after multiplex IHC staining performed on FFPE biopsies collected on cancer patients from EVICTION trial before treatment (Pre) and 28 days post treatment initiation (Post). The data from patients treated with ICT01 monotherapy (group A) or ICT01 plus Pembrolizumab (group C) are shown. Symbols associated with a line correspond to pre- and post-treatment biopsies from the same patient. Quantification of CD3+CD8+PD1+LAG3+TIM3+ cells was done on whole tumor (left panel), in tumor area (middle panel) and in stroma (right panel).

FIG. 4: Effect of blocking anti-LAG-3, -TIM-3 and -TIGIT mAbs on ICT01-mediated activation and expansion of Vγ9Vδ2 T cells in Hu-PBMC
A. Activation (MFI CD25) and expansion (absolute count) of Vγ9Vδ2 T cells, as determined by flow cytometry at day 3 and day 6 respectively in non-activated (NA) and ICT01-stimulated HD Hu-PBMC in presence of 10 μg/mL of blocking mAbs (anti-LAG-3 or anti TIM-3 or anti TIGIT mAb) or corresponding isotype.
B. IFN-γ and Granzyme-B secretion in culture supernatant as determined by Luminex in NA and ICT01-stimulated HD Hu-PBMC in presence of 10 μg/mL of anti-LAG-3 mAb or corresponding isotype.

Wilcoxon tests were used to compare two paired groups *(p<0.05), **(p<0.01) ***(p<0.001) and ****(p<0.0001)

FIG. 5: Effect of blocking anti-LAG-3, -TIM-3 and -TIGIT mAbs on ICT01-mediated killing of tumor cells (SKOV-3) by in vitro expanded Vγ9Vδ2 T cells
A. LAG-3, TIM-3 and TIGIT surface expression on expanded Vγ9Vδ2 T cells determined by Flow cytometry
B. Tumor cell killing by expanded Vγ9Vδ2 T cells as determined by percentage of live target cells by flow cytometry after overnight coculture with unstimulated (NA) and ICT01-activated Vγ9Vδ2 T cells in presence of 10 μg/mL of blocking mAbs (anti-LAG-3 or anti TIM-3 or anti TIGIT mAb) or corresponding isotype (mIgG1)

Wilcoxon tests were used to compare two paired groups *(p<0.05), **(p<0.01) ***(p<0.001) and ****(p<0.0001)

FIG. 6: Effect of blocking anti-LAG-3, anti-TIM-3 and anti-TIGIT mAbs on ICT01-mediated killing of tumor cells (THP1) by in vitro expanded Vγ9Vδ2 T cells
A. Representative flow cytometry profiles of LAG-3, TIM-3 and TIGIT surface expression on expanded Vγ9Vδ2 T cells (upper panel). Representative flow cytometry profiles of BTN3A and LAG-3's ligands (MHC class II), TIM-3's ligands (Galectin 9) and TIGIT's ligands (PVR and Nectin 2) on THP1.
B. Tumor cell killing by in vitro expanded Vγ9Vδ2 T cells as determined by flow cytometry analysis of the percentage of live target cells after 4 hours of coculture with unstimulated (NA) and ICT01-activated Vγ9Vδ2 T cells. The co-cultures were performed in presence of 10 μg/mL of indicated immune-checkpoint blocking mAbs (anti-LAG-3 or anti-TIM-3 or anti-TIGIT) or corresponding isotype control (mIgG1). Wilcoxon tests were used to compare two paired groups *(p<0.05)

FIG. 7: Effect of blocking anti-LAG-3 and anti-TIM-3 mAbs on ICT01-mediated killing of SKOV3 by HD hu-PBMC
A. Tumor cells killing as shown by number of SKOV3 (mean) monitored over time upon coculture with HD hu-PBMC (n=8) performed in presence of ICT01 used at 0.1 μg/mL, or humanized anti-LAG-3 (Relatlimab; 10 μg/mL) versus its corresponding isotype (hIgG4), or ICT01 plus Relatlimab combination.
B. Tumor cells killing as shown by number of SKOV3 (mean) monitored over time upon coculture with HD hu-PBMC (n=8) performed in presence of ICT01 used at 0.1 μg/mL, or humanized anti-TIM-3 (Sabatolimab; 10 μg/mL) versus its corresponding isotype (hIgG4), or ICT01 plus Sabatolimab combination.
   Two-way ANOVA and Holm-Sidak's multiple comparisons tests were used: ****(p<0.0001)

FIG. 8: Effect of soluble LAG-3 Fc on ICT01-mediated activation and expansion of Vγ9Vδ2 T cells in HD hu-PBMC
A. Activation (MFI CD25, upper panel) and expansion (absolute count, lower panel) of Vγ9Vδ2 T cells, as determined by flow cytometry at day 3 and day 6 respectively in non-activated (NA) and ICT01 (0.1 μg/mL)-stimulated HD hu-PBMC in presence of 10 μg/mL of LAG-3 Fc or corresponding control (Ig Fc). Data are shown for each individual donor (n=5).
B. Activation (MFI CD25) of Vγ9Vδ2 T cells as determined by flow cytometry at day 2 in non-activated (NA) and ICT01 (0.03 and 0.1 μg/mL)-stimulated HD hu-PBMC (n=3) cultured with increasing concentrations of LAG-3 Fc (0-10 μg/mL) or its corresponding control (Ig Fc). Data were normalized to the non-treated condition for each individual donor. Individual donor data and Mean±SEM are shown (left panel). The mean fold changes of CD25 MFIs relative to non-treated condition are shown on the heatmap in the right panel.
C. IFN-γ (left panel) and TNF-α (right panel) measured in culture supernatant of day 2 as determined by Luminex in non-activated (NA) and ICT01 (0.03 and 0.1 μg/mL)-stimulated HD hu-PBMC (n=3) cultured with increasing concentration of LAG-3 Fc (0-10 g/mL) or its corresponding control (Ig Fc). Mean values of 3 donors are shown.
D. Activation (MFI CD25) of NK cells, CD4+ and CD8+ T cells as determined by flow cytometry at day 2 in non-activated (NA) and ICT01 (0.1 μg/mL)-stimulated HD hu-PBMC (n=3) cultured with increasing concentrations of LAG-3 Fc (0-10 μg/mL) or its corresponding control (Ig Fc).

FIG. 9: Effect of soluble LAG-3 Fc on ICT01-mediated cytolysis of tumor cells by in vitro expanded Vγ9Vδ2 T cells
A. Representative flow cytometry profiles for MHC class II expression on tumor cell lines (THP1 and SKOV3) and in vitro expanded Vγ9Vδ2 T cells.
B. Flow cytometry assessment of Vγ9Vδ2 T cell degranulation (CD107) after coculture of non-activated or ICT01 (1 μg/mL)-stimulated Vγ9Vδ2 T cells with THP1 in presence of LAG-3 Fc (1 μg/mL) or its corresponding control (Ig Fc). Data is shown for each individual donor (n=6). Wilcoxon tests were used to compare two paired groups *(p<0.05)
C. Flow cytometry assessment of Vγ9Vδ2 T cell degranulation (CD107) after coculture of non-activated or ICT01 (0.1 μg/mL)-stimulated Vγ9Vδ2 T cells with SKOV3 in presence of LAG-3 Fc (1 μg/mL) or its corresponding control (Ig Fc). Data is shown for each individual donor (n=6). Wilcoxon tests were used to compare two paired groups *(p<0.05)

FIG. 10: assessment of direct activity of soluble LAG-3 Fc on MHC class II expressing Vγ9Vδ2 T cells.
A. Activation (MFI CD25) and MHC class II expression as determined by flow cytometry on Vγ9Vδ2 T cells from non-preactivated (NA) or ICT01 (0.1 μg/mL)-preactivated hu-PBMC. Data is shown for each individual donor using different symbols.
B. Activation (MFI CD25) of Vγ9Vδ2 T cells as determined by flow cytometry after a 24-hours culture of total T cells sorted from non-preactivated (NA) and ICT01-preactivated hu-PBMC (n=3) cultured with LAG-3 Fc (1 μg/mL) or its corresponding control (Ig Fc). Data is shown for each individual donor.
C. Representative flow cytometry profiles for MHC class II expression on in vitro expanded Vγ9Vδ2 T cells from 3 donors (left panel), and degranulation (% CD107) of Vγ9Vδ2 T cells after incubation with LAG-3 Fc (1 μg/mL) or corresponding control (Ig Fc). Data is shown for each individual donor.

### DETAILED DESCRIPTION

### Definitions

In order that the present disclosure may be more readily understood, certain terms are first defined. Additional definitions are set forth throughout the detailed description.

The terms “polypeptide”, “protein” or “peptide” as used herein refer to any chain of amino acid residues, regardless of its length or post-translational modification (such as glycosylation).

As used herein, the term “BTN3A” has its general meaning in the art. In specific embodiments, it refers to human BTN3A polypeptides including either BTN3A1 of SEQ ID NO:24, BTN3A2 of SEQ ID NO:25 or BTN3A3 of SEQ ID NO:26.

As used herein, the term “PD-1” has its general meaning in the art and refers to the programmed death-1 receptor. The term “PD-1” also refers to a type I transmembrane protein, belonging to the CD28-B7 signalling family of receptors that includes CD28, cytotoxic T-lymphocyte-associated antigen 4 (CTLA-4), inducible costimulator (ICOS), and B- and T-lymphocyte attenuator (BTLA). In specific embodiments, it refers to human PD-1 of SEQ ID NO: 130 (also found under UniprotKB Accession No. Q15116).

As used herein, PD-L1 (Programmed Death Ligand-1) is one of two cell surface glycoprotein ligands for PD-1 (the other being PD-L2) that downregulate T cell activation and cytokine secretion upon binding to PD-1. In specific embodiments, it refers to human PD-L1 of SEQ ID NO: 131 (also found under UniprotKB Accession No. Q9NZQ7-1).

As used herein, the term “LAG-3” (also named CD223 or FDC protein) is an immune checkpoint inhibitor. In specific embodiments, it refers to human LAG-3 receptor of SEQ ID NO: 127 (also found under UniprotKB Accession No. P18627-1).

As used herein, the term “TIM-3” (also named HAVCR2) is an immune checkpoint inhibitor. In specific embodiments, it refers to human TIM-3 receptor of SEQ ID NO: 128 (also found under UniprotKB Accession No. Q8TDQ0-1).

As used herein, the term “TIGIT” (also called T cell immunoreceptor with Ig and ITIM domains) is an immune checkpoint inhibitor. In specific embodiments, it refers to human TIGIT receptor of SEQ ID NO: 129 (also found under UniprotKB Accession No. Q495A1-1).

The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies. The term “antibody” as used herein also includes bispecific or multispecific molecules. An antibody can be derivatized or linked to another functional molecule, e.g., another peptide or protein (e.g., another antibody or ligand for a receptor) to generate a bispecific molecule that binds to at least two different binding sites or target molecules. The antibody may in fact be derivatized or linked to more than one other functional molecule to generate multi-specific molecules that bind to more than two different binding sites and/or target molecules; such multi-specific molecules are also intended to be encompassed by the term “bispecific molecule” as used herein. To create a bispecific molecule, an antibody of the disclosure can be functionally linked (e.g., by chemical coupling, genetic fusion, noncovalent association or otherwise) to one or more other binding molecules, such as another antibody, antibody fragment, peptide, or binding mimetic, such that a bispecific molecule results. Additionally, for the embodiment in which the bispecific molecule is multi-specific, the molecule can further include a third binding specificity, in addition to the first and second target epitope.

In natural antibodies of rodents and primates, two heavy chains are linked to each other by disulfide bonds, and each heavy chain is linked to a light chain by a disulfide bond. There are two types of light chains, lambda (λ) and kappa (κ). There are five main heavy chain classes (or isotypes) which determine the functional activity of an antibody molecule: IgM, IgD, IgG, IgA and IgE. Each chain contains distinct sequence domains. In typical IgG antibodies, the light chain includes two domains, a variable domain (VL) and a constant domain (CL). The heavy chain includes four domains, a variable domain (VH) and three constant domains (CH1, CH2 and CH3, collectively referred to as CH). The constant region domains of the light (CL) and heavy (CH) chains confer important biological properties such as antibody chain association, secretion, trans-placental mobility, complement binding, and binding to Fc receptors (FcR).

The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate in the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively.

An antigen-binding site, therefore, typically includes six CDRs, comprising the CDRs set from each of a heavy and a light chain V region. Framework Regions (FRs) refer to amino acid sequences interposed between CDRs. Accordingly, the variable regions of the light and heavy chains typically comprise 4 framework regions and 3 CDRs of the following sequence: FR1-CDR1-FR2-CDR2-FR3-CDR3-FR4.

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (Kabat et al., 1992, hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may be used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid residing at the framework positions at which the amino acid is predicted to have a side chain atom within 3A of the CDRs in a three-dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization. In some humanized forms of antibodies, some, most or all of the amino acids outside the CDR regions can be replaced with amino acids from human immunoglobulin molecules but where some, most or all amino acids within one or more CDR regions are unchanged. Small additions, deletions, insertions, substitutions, or modifications of amino acids are permissible as long as they would not abrogate the desired property of the parent antibody.

Suitable human immunoglobulin molecules would include IgG1, IgG2, IgG3, IgG4, IgA and IgM molecules. A “humanized” antibody preferably retains a similar antigenic specificity, such as binding affinity, as the original antibody. However, using certain methods of humanization, the affinity and/or specificity of binding of the antibody may be increased using methods of “directed evolution”, as described by Wu et al., Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

The term “antigen-binding fragment” of an antibody (or simply “antibody fragment”), as used herein, refers to full length or to one or more fragments of an antibody that retain the ability to specifically bind to an antigen (e.g., a BTN3A protein, a LAG-3 receptor, a TIM-3 receptor, or a TIGIT receptor, as above defined). In specific embodiments, an antibody provided herein is an antibody fragment, and more particularly any protein including an antigen-binding domain of an antibody as disclosed herein. Well known antibody fragments comprise: a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989 Nature 341:544-546), which consists of a VH domain, or any fusion proteins comprising such antigen-binding fragments; a diabody, which refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Furthermore, although the two domains of the Fv fragment, VL and VH, are coded for by separate genes, they can be joined, using recombinant methods, by a synthetic linker that enables them to be made as a single chain protein in which the VL and VH regions pair to form monovalent molecules (known as single chain Fv (scFv); see e.g., Bird et al., 1988 Science 242:423-426; and Huston et al., 1988 Proc. Natl. Acad. Sci. 85:5879-5883). Such single chain antibodies are also intended to be encompassed within the term “antigen-binding fragment” of an antibody (also shortly named herein antibody fragment). More generally antibody fragments as herein intended also encompass single-domain antibodies that are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1). These antibody fragments are obtained using conventional techniques known to those of skill in the art, and the fragments are screened for utility in the same manner as are intact antibodies. Well-suited antibody fragments include, but are not limited to, Fv, Fab, F(ab′)2, Fab′, dsFv, scFv, sc(Fv)2 and diabodies. Antibody fragments can be made by various techniques, including but not limited to proteolytic digestion of an intact antibody as well as production by recombinant host cells as described herein.

The term “monoclonal antibody” as used herein refers to a preparation of antibody molecules of single specificity. A monoclonal antibody displays a single binding specificity and affinity for a particular epitope. Accordingly, the term “human monoclonal antibody” refers to an antibody displaying a single binding specificity which has variable and constant regions derived from or based on human germline immunoglobulin sequences or derived from completely synthetic sequences. The method of preparing the monoclonal antibody is not relevant for the binding specificity.

“Recombinant antibodies” are antibodies which are produced, expressed, generated or isolated by recombinant means, such as antibodies which are expressed using a recombinant expression vector transfected into a host cell; antibodies isolated from a recombinant combinatorial antibody library; antibodies isolated from an animal (e.g. a mouse) which is transgenic due to human immunoglobulin genes; or antibodies which are produced, expressed, generated or isolated in any other way in which particular immunoglobulin gene sequences (such as human immunoglobulin gene sequences) are assembled with other DNA sequences. Recombinant antibodies include, for example, chimeric and humanized antibodies. In some embodiments a recombinant human antibody of this disclosure has the same amino acid sequence as the corresponding naturally occurring human antibody but differs structurally from said naturally occurring human antibody. For example, in some embodiments the glycosylation pattern is different as a result of the recombinant production of the recombinant human antibody. In some embodiments the recombinant human antibody is chemically modified by addition or subtraction of at least one covalent chemical bond relative to the structure of the human antibody that occurs naturally in humans.

An “isolated antibody”, as used herein, refers to an antibody that is substantially free of other antibodies having different antigenic specificities (e.g., an isolated antibody that specifically binds to BTN3A is substantially free of antibodies that specifically bind to other antigens than BTN3A). An isolated antibody that specifically binds to BTN3A may, however, have cross-reactivity to other antigens, such as related BTN3A molecules from other species. Moreover, an isolated antibody may be substantially free of other cellular material and/or chemicals.

The phrases “an antibody recognizing an antigen” and “an antibody having specificity for an antigen” are used interchangeably herein with the term “an antibody which binds specifically to an antigen”. The term “anti-LAG-3 antibody” are also shortly used herein with the meaning of “an antibody recognizing LAG-3”. The term “BTN3A antibody” are also shortly used herein with the meaning of “an antibody recognizing BTN3A”.

As used herein, the term “activating antibody” refers to an antibody able to directly or indirectly induce immune functions of effector cells. In particular, as used herein, a BTN3A activating antibody has at least the capacity to induce the activation of γδ T cells, typically Vγ9Vδ2 T cells, in co-culture with BTN3A expressing cells, with an EC50 below 5 μg/ml, preferably of 1 μg/ml or below, as measured in a degranulation assay (see for detailed assay WO2020/025703).

As used herein, the term “binding” in the context of the binding of an antibody to a predetermined antigen or epitope, notably BTN3A, LAG-3, TIM-3 or TIGIT, means typically a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less when determined by for instance surface plasmon resonance (SPR) technology in a BIAcore 3000 instrument using typically a soluble form of the antigen as the ligand and the antibody as the analyte. BIACORE® (GE Healthcare, Piscaataway, NJ) is one of a variety of surface plasmon resonance assay formats that are routinely used to epitope bin panels of monoclonal antibodies. Typically, an antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} for binding to a non-specific antigen (e.g., BSA, casein), which is not identical or closely related to the predetermined antigen. When the K_{D} of the antibody is very low (that is, the antibody has a high affinity), then the K_{D} with which it binds the antigen is typically at least 10,000-fold lower than its K_{D} for a non-specific antigen.

The term “affinity”, as used herein in the context of an antibody, means the strength of the binding of an antibody to an epitope.

The term “Kₒₙ” or “Kass” (Kₐ), as used herein, is intended to refer to the association rate of a particular antibody-antigen interaction, whereas the term “K_{dis}” (Kd) or “K_{off},” as used herein, is intended to refer to the dissociation rate of a particular antibody-antigen interaction.

The term “K_{D}”, as used herein, is intended to refer to the equilibrium dissociation constant, which is obtained from the ratio of k_{off} to kₒₙ (i.e. k_{off}/kₒₙ) and is expressed as a molar concentration (M). The K_{D} value relates to the concentration of antibody (the amount of antibody needed for a particular experiment) and so the lower the K_{D} value (lower concentration) and thus the higher the affinity of the antibody. K_{D} values for antibodies can be determined using methods well established in the art. Preferred methods for determining the K_{D} values of mAbs can be found in Harlow, et al., Antibodies: A Laboratory Manual, Cold Spring Harbor Laboratory Press, Cold Spring Harbor, N.Y., (1988), Coligan et al., eds., Current Protocols in Immunology, Greene Publishing Assoc, and Wiley Interscience, N.Y., (1992, 1993), and Muller, Meth. Enzymol. 92:589-601 (1983), which references are entirely incorporated herein by reference. A method for determining the K_{D} of an antibody is by using surface plasmon resonance, or by using a biosensor system such as a Biacore® (see also for detailed information regarding affinity assessment Rich R L, Day Y S, Morton T A, Myszka D G. High-resolution and high-throughput protocols for measuring drug/human serum albumin interactions using BIACORE®. Anal Biochem. 2001 Sep. 15; 296(2):197-207) or Octet® systems. The Octet® platform is based on bio-layer interferometry (BLI) technology. The principle of BLI technology is based on the optical interference pattern of white light reflected from two surfaces—a layer of immobilized protein and an internal reference layer. The binding between a ligand immobilized on the biosensor tip surface and an analyte in solution produces an increase in optical thickness at the biosensor tip, which results in a shift in the interference pattern measured in nanometers. The wavelength shift (AA) is a direct measure of the change in optical thickness of the biological layer, when this shift is measured over a period of time and its magnitude plotted as a function of time, a classic association/dissociation curve is obtained. This interaction is measured in real-time, allowing to monitor binding specificity, association rate and dissociation rate, and concentration (see Abdiche et al. 2008 but also the details in the results). Affinity measurements are typically performed at 25° C.

As used herein, the term “specificity” refers to the ability of an antibody to detectably bind an epitope presented on an antigen, such as a BTN3A, LAG-3, TIM-3 or TIGIT.

In some embodiments, an antibody having specificity for BTN3A is intended to refer to an antibody that binds to human BTN3A as expressed on peripheral blood mononuclear cells (PBMCs), preferably with an EC₅₀ below 50 μg/ml and more preferably below 10 μg/ml as determined in the assays disclosed in WO2020/025703 which content is incorporated herein by reference, in particular with reference to Table 4. In other embodiments, it binds to an antigen recombinant polypeptide with a K_{D} of 100 nM or less, 10 nM or less, 1 nM or less, 100 μM or less, or 10 μM or less, as measured by SPR measurements as mentioned above but see also for details Table 4 of WO2020/025703).

An antibody that “cross-reacts with an antigen other than BTN3A” is intended to refer to an antibody that binds that antigen other than BTN3A with a K_{D} of 10 nM or less, 1 nM or less, or 100 μM or less. An antibody that “does not cross-react with a particular antigen” is intended to refer to an antibody that binds to that antigen, with a K_{D} of 1 μM or greater, or a K_{D} of 10 μM or greater. In certain embodiments, such antibodies that do not cross-react with the antigen exhibit essentially undetectable binding against these proteins in standard binding assays. In specific embodiments, the humanized antibody of the present disclosure, e.g., mAb1, cross-reacts with cynomolgus BTN3A1, BTN3A2 and BTN3A3 of SEQ ID NO:27, SEQ ID NO:28 and SEQ ID NO:29 respectively for example as measured in Biacore assay (see notably the related assay exemplified in WO/2020/025703 with reference to Table 26).

The term “inhibition,” “inhibitor,” or “antagonist” includes a reduction in a certain parameter, e.g., an activity, of a given molecule, e.g., an immune checkpoint inhibitor. For example, inhibition of an activity, e.g., an activity of a given molecule, e.g., an inhibitory molecule, of at least or about 5%, 10%, 20%, 30%, 40%, 50%, 60%, 70%, 80%, 90%, 95%, or more is included by this term. Thus, inhibition needs to not be 100%.

A “fusion protein” and a “fusion polypeptide” refer to a protein or polypeptide having at least two portions covalently linked together, where each of the portions is a polypeptide having a different property. The property may be a biological property, such as activity in vitro or in vivo. The property can also be a simple chemical or physical property, such as binding to a target molecule, catalysis of a reaction, etc. The two portions can be linked directly by a single peptide bond or through a peptide linker, but are in reading frame with each other. Examples of fusion protein includes in particular receptor binding domain fused to immunoglobulin constant part (such as Fc or Ig fusion protein).

Specificity can further be exhibited by, e.g., an about 10:1, about 20:1, about 50:1, about 100:1, 10.000:1 or greater ratio of affinity/avidity in binding to the specific antigen versus nonspecific binding to other irrelevant molecules (in this case the specific antigen is a BTN3A polypeptide, LAG-3, TIM-3 or TIGIT receptor respectively or alternatively MHC class II in the case of soluble hLAG-3 Ig molecule).

As used herein, the term “Avidity” refers to an informative measure of the overall stability or strength of the antibody-antigen complex. It is controlled by three major factors: antibody epitope affinity; the valence of both the antigen and antibody; and the structural arrangement of the interacting parts. Ultimately these factors define the specificity of the antibody, that is, the likelihood that the particular antibody is binding to a precise antigen epitope.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc.

As used herein, the term, “optimized” means that a nucleotide sequence has been altered to encode an amino acid sequence using codons that are preferred in the production cell or organism, generally a eukaryotic cell, for example, a Chinese Hamster Ovary cell (CHO) or a human cell. The optimized nucleotide sequence is engineered to retain completely or as much as possible the amino acid sequence originally encoded by the starting nucleotide sequence. The amino acid sequences encoded by optimized nucleotide sequences are also referred to as optimized.

The term “identity” as used herein in reference to polypeptide sequences, refers to the amino acid sequence identity between two molecules. When an amino acid position in both molecules is occupied by the same amino acid, then the molecules are identical at that position. The identity between two polypeptides is a direct function of the number of identical positions. In general, the sequences are aligned so that the highest order match is obtained (including gaps if necessary). The percent identity between the two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two amino acid sequences can be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. Alternatively, the percent identity between two amino acid sequences can be determined using published techniques and widely available computer programs, such as BLASTP, FASTA (Atschul et al., J. Molecular Biol. 215:403, 1990), or the Needleman and Wunsch (J. Mol, Biol. 48:444-453, 1970) algorithm which has been incorporated into the GAP program in the GCG software package (Devereux et al., Nucleic Acids Res. 12:387, 1984, typically available at http://www.gcg.com), using either a Blossom 62 matrix or a PAM250 matrix, and a gap weight of 16, 14, 12, 10, 8, 6, or 4 and a length weight of 1, 2, 3, 4, 5, or 6.

The percent identity between two nucleotide amino acid sequences may also be determined using for example algorithms such as the BLASTN program for nucleic acid sequences using as defaults a word length (W) of 11, an expectation (E) of 10, M=5, N=4, and a comparison of both strands.

Additional antibodies can be identified based on their ability to cross-compete (e.g., to competitively inhibit the binding of, in a statistically significant manner) with other antibodies of the disclosure in standard antigen binding assays such as an ELISA binding assay. The ability of a test antibody to inhibit the binding of antibodies of the present disclosure to the target demonstrates that the test antibody can compete with that antibody for binding to the target; such an antibody may, according to non-limiting theory, bind to the same or a related (e.g., a structurally similar or spatially proximal) epitope on the target as the antibody with which it competes. Thus, another aspect of the disclosure provides antibodies that bind to the same antigen as, and compete with, the antibodies disclosed herein. As used herein, an antibody “competes” for binding when the competing antibody inhibits the target binding of an antibody or antigen binding fragment of the disclosure by more than 50, 51, 52, 53,54,55,56,57,58, 59,60,61,62,63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% in the presence of an equimolar concentration of competing antibody.

“combination therapy”, “co-administration”, “combined administration” or “concomitant administration” refers to a combined administration of at least two therapeutic agents, where a first agent, typically a BTN3A activating compound is administered at the same time or separately within time intervals, with a second agent, e.g. an immune checkpoint inhibitor selected from LAG-3 inhibitors, TIM-3 inhibitors or TIGIT inhibitors, or their combinations, and optionally a third agent, e.g. PD-1/PD-L1 inhibitor, or IL2 or IL15 cytokine or their derivatives, in the same subject in need thereof, where these time intervals allow that the combined partners show a cooperative or synergistic effect for treating a disorder, e.g. cancer.

Examples of cancers that may be treated using the methods of the invention include skin cancer, cutaneous or intraocular malignant melanoma, head or neck cancer, breast cancer, lung cancer, renal cancer, bladder cancer, liver cancer, bone cancer, pancreatic cancer, uterine cancer, ovarian cancer, colorectal cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the kidney or ureter, cancer of the penis, neoplasm of the central nervous system (CNS), brain stem glioma, pituitary adenoma, Kaposi's sarcoma, epidermoid cancer, solid tumors of childhood, hematologic malignancies including for example Hodgkin lymphoma/primary mediastinal B-cell lymphoma, non-Hodgkin's lymphoma including B-cell lymphoma, multiple myeloma, follicular lymphoma, diffuse large B-cell lymphoma, Burkitt's lymphoma, immunoblastic large cell lymphoma, precursor B-lymphoblastic lymphoma, mantle cell lymphoma, T-cell lymphoma for example mycosis fungoides, anaplastic large cell lymphoma, precursor T-lymphoblastic lymphoma, chronic or acute leukemias including acute myeloid leukemia, chronic myeloid leukemia, acute lymphoblastic leukemia, chronic lymphocytic leukemia, and any combinations of said cancers.

The present invention is also applicable to treatment of metastatic, refractory or recurrent malignancies. It is not intended to imply that the therapeutic agents must be administered at the same time and/or formulated for delivery together although these methods of delivery are within the scope described herein. The BTN3A activating antibody, e.g. ICT01 as herein disclosed, can be administered concurrently with or prior to, or subsequent to one or more other additional therapies or therapeutic agents. The terms are also meant to encompass treatment regimens in which the agents are not necessarily administered by the same route of administration.

As used herein the term synergy or synergistic effect when used in connection with a description of the efficacy of a combination of agents, means any measured effect of the combination which is greater than the effect predicted from a sum of the effects of the individual agents (i.e., greater than an additive effect). In some embodiments, the rate of tumor growth or tumor size (e.g., the rate of change of the size (e.g., volume, mass) of the tumor) is used to determine whether a combination of drugs is synergistic (e.g., the combination of drugs is synergistic when the rate of tumor growth is slower than would be expected if the combination of drugs produced an additive effect). In some embodiments, median overall survival time (e.g. of <12 months) is used to determine whether a combination of drugs is synergistic (e.g., a combination of drugs is synergistic when the median overall survival time of a subject or population of subjects is longer than would be expected if the combination of drugs produced an additive effect). In some embodiments, γδ T cell activation can also be used to determine whether a combination of drugs is synergistic (e.g., a combination of drugs is synergistic when the activation rate (determined as increased expression of activation markers compared to a baseline value) of a specific γδ T cell subset (typically Vγ9Vδ2 T cell subset) is higher than would be expected if the combination of drugs produced an additive effect). In some embodiments, γδ T cell proliferation and expansion can also be used to determine whether a combination of drugs is synergistic (e.g., a combination of drugs is synergistic when the proliferation or expansion rate (determined as increased percentage of the population or increased absolute cell number compared to a baseline value) of a specific γδ T cell subset (typically Vγ9δ2 T cell subset) is higher than would be expected if the combination of drugs produced an additive effect). In some embodiments, γδ T cell cytotoxicity can also be used to determine whether a combination of drugs is synergistic (e.g., a combination of drugs is synergistic when the cytotoxic capacity (determined as increased percentage of degranulation in effector cells or increased percentage of apoptosis in target cells or decreased percentage and/or absolute cell number of live cells in target cells compared to a baseline value) of a specific T cell subset (typically Vγ9δ2 T cell subset) is higher than would be expected if the combination of drugs produced an additive effect).

### BTN3A Activating Antibody

A BTN3A activating antibody according to the present disclosure typically exhibits one or more of the following properties:
(i) it binds to BTN3A with a K_{D} of 10 nM or lower, preferably with a K_{D} of 1 nM or lower, as measured by SPR, for example as described in the Examples of patent application WO2020/025703;
(ii) it binds to human PBMCs with an EC₅₀ of 50 μg/ml or below, preferably of 10 μg/ml or below, as measured in a flow cytometry assay as described in the Examples of patent application WO2020/025703;
(iii) it induces the activation of γδ T cells, typically Vγ9Vδ2 T cells, in co-culture with BTN3A expressing cells, with an EC₅₀ below 5 μg/ml, preferably of 1 μg/ml or below, as described in the Examples of patent application WO2020/025703; and/or,
(iv) it induces in vitro activation of Vγ9Vδ2 T cells in human peripheral blood mononuclear cells (PBMC), with an EC₅₀ below 0.1 μg/mL, preferably of 0.01 μg/mL or below, e.g., between 100 pg/mL and 0.1 μg/mL, as measured by surface expression of the activation markers CD69, and,
(v) optionally, it cross-reacts to cynomolgus BTN3A with a K_{D} of 100 nM or lower, preferably with a K_{D} of 10 nM or lower, as measured by SPR, for example as described in the Examples of patent application WO2020/025703.

Preferably, as used herein, the BTN3A activating antibodies for use according to the present disclosure have at least the above properties (i) and (iv).

Specific examples of BTN3A activating antibodies are described in the paragraphs below. In some embodiments, a BTN3A activating antibody is selected from the group consisting of BTN3A antibodies which are described in the International Patent Applications WO2012080769; WO2012080351, and WO2020025703. In some particular embodiments, a BTN3 activating antibody is selected from the humanized antibodies described in WO2020025703 or is a humanized version of the BTN3A agonist antibodies described in WO2012080769 and WO2012080351. In some embodiments, the BTN3A antibody is selected from mAb 20.1, and mAb 7.2, which are obtainable from one of the hybridomas accessible under CNCM deposit number 1-4401, and I-4402 such as described in WO2012080769 and WO2012080351 or humanized version thereof, as well as from humanized mAbs 1-6 described in WO2020/025703.

In some embodiments, the BTN3A activating antibody comprises the six CDRs (CDR1 (also called HCDR1), VH CDR2 (also called HCDR2), VH CDR3 (also called HCDR3), VL CDR1 (also called LCDR1), VL CDR2 (also called LCDR2), VL CDR3 (also called LCDR3)) of the antibodies 20.1, or 7.2 described in WO2012/080769 and WO2012080351 or mAbs 1-6 as described in WO2020/025703. In particular embodiments, the BTN3A activating antibody comprises HCDR1, HCDR2, HCDR3, LCDR1, LCDR2, and LCDR3 as shown in Table 1 below:

TABLE 1
CDR regions of mAb1, mAb2, mAb4 and mAb5, parental murine mAb
7.2 and murine mAb 20.1 antibody according to Kabat numbering as
defined in WO2020025703.

| Original | | | | | | |
|---|---|---|---|---|---|---|
| antibody | HCDR1 | HCDR2 | HCDR3 | LCDR1 | LCDR2 | LCDR3 |
| mAb 7.2 | SEQ ID | SEQ ID | SEQ ID | SEQ ID | SEQ ID | SEQ ID |
| ICT01 (mAb1) | NO: 12 | NO: 13 | NO: 14 | NO: 15 | NO: 16 | NO: 17 |
| mAb2 | | | | | | |
| mAb4 | | | | | | |
| mAb5 | | | | | | |
| mAb 20.1 | SEQ ID | SEQ ID | SEQ ID | SEQ ID | SEQ ID | SEQ ID |
| | NO: 18 | NO: 19 | NO: 20 | NO: 21 | NO: 22 | NO: 23 |

In some embodiments of antibodies as herein disclosed, the 6 CDR regions are 100% identical to the 6 CDR regions of the antibodies 20.1, or 7.2 described in WO2012/080769 and WO2012/080351 or of mAbs 1-6 described in WO2020/025703, notably in some embodiments, the 6 CDR regions of antibodies as herein disclosed are 100% identical to the 6 CDR regions of Table 1, notably of mAb 7.2, mAb 1, mAb 2, mAb 4, and mAb 5.

Other antibodies as disclosed herein include those having amino acids that have been mutated by amino acid deletion, insertion or substitution, yet have at least 60, 70, 80, 90, 95, 96, 97, 98, 99 or 100 percent identity in the CDR regions as compared to the 6 CDR regions of the antibodies 20.1, or 7.2 described in WO2012/080769 and WO2012/080351 or of mAbs 1-6 described in WO2020/025703, notably as compared to the 6 CDR regions defined in Table 1. In some embodiments, as per the present disclosure, antibodies may have between 1, 2, 3 or 4 amino acid variations (including deletion, insertion, or substitution) in one or more CDRs, as compared to the CDR sequences of the antibodies 20.1, or 7.2 described in WO2012/080769 and WO2012/080351 or of mAbs 1-6 described in WO2020/025703, notably as compared to the CDR sequences of Table 1, more particularly as compared to the CDR sequences of mAb 7.2, mAb1, mAb2, mAb4 and mAb5.

Antibodies of the present disclosure include also those having at least 90%, notably at least 95, 96, 97, 98, 99 or 100% identity with the VH and VL regions as defined in Table 2. More particularly, antibodies of the disclosure include the selected humanized recombinant antibodies mAb1, mAb2, mAb4 and mAb5, which are structurally characterized by their variable heavy and light chain amino acid sequences and human constant regions (isotypes) as described in the Table 2 below:

TABLE 2
Variable heavy and light chain amino acid sequences of mAb1-mAb6

| | VH | VL | |
|---|---|---|---|
| | Amino acid | Amino acid | |
| Antibody | sequence | sequence | Isotype constant region |
| ICT01 | SEQ ID NO: 1 | SEQ ID NO: 2 | Silent IgG1 |
| (mAb1) | (VH2 7.2) | (Vk1 7.2) | L247F/L248E/P350S |
| mAb2 | SEQ ID NO: 1 | SEQ ID NO: 3 | Silent IgG1 |
| | (VH2 7.2) | (Vk2 7.2) | L247F/L248E/P350S |
| mAb3 | SEQ ID NO: 125 | SEQ ID NO: 126 | Silent IgG1 |
| | (VH humanized | (VL humanized | L247F/L248E/P350S |
| | mab 20.1) | mab 20.1 | |
| mAb4 | SEQ ID NO: 1 | SEQ ID NO: 2 | IgG4 S241P/L248E |
| | (VH2 7.2) | (Vk1 7.2) | |
| mAb5 | SEQ ID NO: 1 | SEQ ID NO: 3 | IgG4 S241P/L248E |
| | (VH2 7.2) | (Vk2 7.2) | |
| mAb6 | SEQ ID NO: 125 | SEQ ID NO: 126 | IgG4 S241P/L248E |
| | (VH humanized | (VL humanized | |
| | mab 20.1) | mab 20.1 | |

mAb3 and mAb6 are humanized antibodies of parental murine BTN3A activating antibody, referred as mAb 20.1 described in WO2012/080351.

The corresponding amino acid and nucleotide coding sequence of the constant isotype regions of IgG1, IgG4 and their mutant versions IgG1 L247F/L248E/P350S and IgG4 S241P/L248E used for generating mAb1 to mAb6 are well-known in the art (Oganesyan et al., Acta Crystallogr D Biol Crystallogr. 2008 June; 64 (Pt 6): 700-4; Reddy et al., J Immunol. 2000 Feb. 15; 164(4):1925-33). The C-terminal lysine found in IgG may be naturally cleaved off and this modification does not affect the properties of the antibody; so, this residue may additionally be deleted in the constructs of mAb1 to mAb6.

Full length light and heavy chains and corresponding coding sequences of mAb1, mAb2, mAb4 and mAb 5 are shown in the Table 3 below.

TABLE 3
Full length heavy and light chain DNA coding sequences

| Antibody | Amino acid sequence | DNA coding sequence |
|---|---|---|
| ICT01 | Heavy Chain: SEQ ID NO: 4 | Heavy Chain: SEQ ID NO: 8 |
| (mAb1) | Light Chain: SEQ ID NO: 6 | Light Chain: SEQ ID NO: 10 |
| mAb2 | Heavy Chain: SEQ ID NO: 4 | Heavy Chain: SEQ ID NO: 8 |
| | Light Chain: SEQ ID NO: 7 | Light Chain: SEQ ID NO: 11 |
| mAb4 | Heavy Chain: SEQ ID NO: 5 | Heavy Chain: SEQ ID NO: 9 |
| | Light Chain: SEQ ID NO: 6 | Light Chain: SEQ ID NO: 10 |
| mAb5 | Heavy Chain: SEQ ID NO: 5 | Heavy Chain: SEQ ID NO: 9 |
| | Light Chain: SEQ ID NO: 7 | Light Chain: SEQ ID NO: 11 |

Other BTN3A activating antibodies, in particular variant of mAb20.1 activating antibodies are disclosed in WO2023/161457 (Evobright GmbH) which content is incorporated herein in its entirety.

A preferred exemplary BTN3A activating antibody for use according to the present disclosure is ICT01, as defined by its heavy chain of SEQ ID NO:4 and its light chain of SEQ ID NO:6.

In certain embodiments that may be combined with the previous embodiments, an antibody provided herein is an antibody fragment of the above-defined antibodies. Antibody fragments include for example, but are not limited to, Fab, Fab′, Fab′-SH, F(ab′) 2, Fv, Unibody, and scFv fragments, diabodies, single domain or nanobodies and other fragments. Preferably, it is a monovalent antibody, such as a Fab of scFv fragments.

In some embodiments, the antibodies of the present disclosure compete for binding to BTN3 antibodies described above, in particular an antibody of the present disclosure competes for binding with an antibody selected from mAb 20.1, and mAb 7.2, which are obtainable from one of the hybridomas accessible under CNCM deposit number 1-4401, and I-4402 such as described in WO2012080769 and WO2012080351, as well as from mAbs 1-6 described in WO2020025703. In more particular embodiments, the antibodies of the present disclosure compete for binding with an antibody selected from mAb 7.2 as produced by the hybridomas deposited at the CNCM under deposit number I-4402, and an ICT01 antibody having a heavy chain of SEQ ID NO:4 and a light chain of SEQ ID NO:6.

In some embodiments, antibodies for use according to the present disclosure are chimeric, humanized, or human antibodies. In preferred embodiments of the present disclosure, the BTN3A antibody is a humanized antibody. Typically, a non-human antibody is humanized to reduce immunogenicity to humans, while having at least the same affinity (or superior affinity) of the parental non-human antibody. More particularly the BTN3A antibody is a humanized form of the murine antibodies mAb 20.1, or 7.2 disclosed in WO2012/080351. In preferred embodiments, the BTN3A antibodies for use of the present disclosure are humanized antibodies of the parent antibody mAb 7.2 as disclosed in WO2012/080351.

Generally, a humanized antibody comprises one or more variable domains in which, CDRs, (or portions thereof) are derived from a non-human antibody, e.g., the murine mAbs 7.2, and FRs (or portions thereof) are derived from the murine antibody sequences with mutations to reduce immunogenicity. A humanized antibody optionally will also comprise at least a portion of a human constant region. Preferably, the recombinant antibody for use according to the disclosure is a humanized silent antibody, typically a humanized silent IgG1 or IgG4 antibody. Well-suited humanized anti-BTN3A antibodies for use according to the present disclosure are typically described in WO2020/025703 and include mAbs having VH/VL polypeptides sequences of Table 2 and mAbs having light/heavy chains of Table 3.

As used herein, the term “silent” antibody refers to an antibody that exhibits no or low FcγR binding and/or C1q binding as measured in binding assays such as those described in WO2020/025703. In one embodiment, the term “no or low FcγR and/or C1q binding” means that the silent antibody exhibits an FcγR and/or C1q binding that is at least below 50%, for example below 80% of the FcγR and/or C1q binding that is observed with the corresponding antibody with wild type human IgG1 or IgG4 isotype.

### LAG-3 Inhibitors

In certain embodiments, the combination therapy comprises administering a therapeutically effective amount of a BTN3A activating antibody as described above, e.g., ICT01, in combination with a therapeutically effective amount of a LAG-3 inhibitor.

LAG-3 inhibitors which may be used in the combination therapy as disclosed herein include without limitation anti-LAG-3 antibodies or recombinant LAG-3 proteins.

In some embodiments, an anti-LAG-3 antibody molecule is intended to refer to a monospecific antibody molecule or a bispecific antibody molecule which exhibits one or more of the following properties:
(i) it specifically binds to LAG-3, e.g. human LAG-3. The binding specificity of Ab can be determined in flow cytometry assays by monitoring binding of the antibody to cells expressing LAG-3 protein (e.g. transfected cells or primary immune cells) preferably with an EC50 below 100 nM and more preferably below 10 nM. Additionally, or alternatively, the antibody binds to a recombinant human LAG-3 protein with high affinity, with a K_{D} of 100 nM or less, 10 nM or less, 1 nM or less or 100 μM or less, as measured by SPR, BLI or other suitable binding assays;
(ii) it inhibits binding of LAG-3 to its ligands, e.g. major histocompatibility-class (MHC) II molecules with an IC50 below 100 nM and more preferably below 10 nM;
(iii) it modulates (e.g. stimulates, enhances, or restores) an immune response. The ability of the antibody to modulate an immune response can be indicated by one or more of: enhancing antigen-specific T cell responses; enhancing T cell activation and/or proliferation; enhancing cytokine (e.g; IL-2, IFN-g) and/or chemokine secretion, enhancing T cell homeostasis, enhancing tumor infiltrating lymphocytes; reducing suppressor activity of Tregs or delaying tumor growth (e.g. in an in vivo model).
(iv) Optionally, it may also bind to a non-human primate LAG-3, e.g., cynomolgus LAG-3.

In one embodiment, the LAG-3 inhibitor for use in the combination therapy is an anti-LAG-3 antibody molecule. In some embodiments, the LAG-3 inhibitor is chosen from Relatlimab. In some embodiments, the LAG-3 inhibitor is chosen from Favezelimab (also named as MK-4280) (Merck Sharp & Dohme). In some embodiments, the anti-LAG-3 antibody comprises the VH of SEQ ID NO:52 and the VL of SEQ ID NO:53 or functional variants with VH and VL having at least 90%, 95%, or at least 98% identity to SEQ ID NO:52 and SEQ ID NO:53, respectively In some embodiments, the anti-LAG-3 antibody comprises a heavy chain of SEQ ID NO:50 and a light chain of SEQ ID NO:51, or functional variants with heavy and light chains having at least 90%, 95%, or at least 98% identity to SEQ ID NO:50 and SEQ ID NO:51, respectively. Other anti-LAG-3 antibody molecules are disclosed in WO2016/028672 (which content is incorporated herein in its entirety). In some embodiments, the anti-LAG-3 antibody comprises the 6 CDRs (HCDR1, HCDR2, HCDR3, LCDR1, LCDR2, LCDR3) of SEQ ID NO:54-59 respectively.

Other anti-LAG-3 antibody molecules are disclosed in WO2016/028672 (which content is incorporated herein in its entirety).

In one embodiment, the LAG-3 inhibitor for use in the combination therapy with BTN3A activating antibody, e.g. ICT01, is a fixed dose combination of an anti-LAG-3 antibody and anti-PD-1 antibody. In some embodiments, the LAG-3 inhibitor is chosen from Nivolumab/Relatlimab (sold under the brand name of Opdualag, BMS), a fixed dose combination of nivolumab (anti-PD-1 antibody) and relatlimab (anti-LAG-3 antibody). Other examples of fixed dose combinations are disclosed in WO2020/081928. In some embodiments, the LAG-3 inhibitor is a fixed dose combination of anti-LAG-3 antibody with heavy and light chains of SEQ ID NO:33 and 34, or their variant sequences with at least 90%, 95% or at least 98% identity to SEQ ID NO:33 and SEQ ID NO: 34 respectively and an anti-PD-1 antibody with heavy and light chains of SEQ ID NO:31 and 32, or their variant sequences with at least 90%, 95% or at least 98% identity to SEQ ID NO:31 and SEQ ID NO:32 respectively. In some embodiments, the anti-LAG-3 antibody comprises the VH and VL of SEQ ID NO: 37 and SEQ ID NO:38 respectively, or related anti-LAG-3 antibody with VH and VL having at least 90%, 95% or at least 98% identity to SEQ ID NO:37 and SEQ ID NO:38, respectively.

In some embodiments, the LAG-3 inhibitor is an anti-LAG-3 antibody. In certain embodiments, the anti-LAG-3 antibody is a full-length antibody. In certain embodiments, the antibody is a multispecific antibody, e.g., a dual-affinity re-targeting antibody (DART), a DVD-Ig, or bispecific antibody. In some embodiments, the anti-LAG-3 antibody is BMS-986016, IMP731 (H5L7BW), MK-4280 (favezelimab), REGN3767, GSK2831781, humanized BAP050, IMP-701 (LAG-525), aLAG3 (0414), aLAG3 (0416), Sym022, TSR-033, TSR-075, XmAb22841, MGD013, B1754111, FS118, P 13B02-30, or AVA-017.

In one embodiment, the LAG-3 inhibitor for use in the combination therapy with BTN3A activating antibody, e.g. ICT01, is tebotelimab (Macrogenics), a bispecific DART molecule designed to independently or coordinately block PD-1 and LAG-3 checkpoint molecules. In some embodiments, related bispecific DART molecules are described in WO2016/200782 (which content is incorporated herein by reference in its entirety). In some embodiments, the anti-LAG-3 antibody is a bispecific DART molecule comprising a first polypeptide of SEQ ID NO: 60 and a second polypeptide of SEQ ID NO:61, or their variant sequences with at least 90%, 95% or at least 98% identity to SEQ ID NO:60 and SEQ ID NO:61 respectively. In some embodiments, the anti-LAG-3 antibody is a bispecific DART molecule comprising a first VH sequence of SEQ ID NO:62 and second VH sequence of SEQ ID NO: 64, and a first VL sequence of SEQ ID NO:63 and a second VL sequence of SEQ ID NO: 132, or their variant sequences with at least 90%, 95% or at least 98% identity to SEQ ID NO:62, SEQ ID NO:63, SEQ ID NO:64 and SEQ ID NO: 132 respectively. In some embodiments, the anti-LAG-3 antibody is a bispecific DART molecule comprising a first VH sequence with 3 CDRs of SEQ ID NOS: 65-67 respectively, a first VL sequence with 3 CDRs of SEQ ID NO:68-70 respectively, a second VH sequence with 3 CDRs of SEQ ID NO:71-73, and a second VL sequence with 3 CDRs of SEQ ID NO:74-76.

In some embodiments, a recombinant LAG-3 protein for use in the combination therapy as herein disclosed, refers to the whole LAG-3 protein or derivatives comprising mutants, variants or fragments of LAG-3 or a recombinant soluble human LAG-3-Ig fusion protein (also referred as LAG-3 Fc fusion protein), provided that said recombinant LAG-3 protein exhibits one or more of the following properties:
(i) it specifically binds to human and murine MHC class II. The binding specificity can be determined in flow cytometry assays by monitoring binding of the recombinant LAG-3 protein or derivatives to antigen presenting cells from human or murine origin expressing MHC class II (e.g. transfected cells or primary immune cells) preferably with an EC50 below 100 nM and more preferably below 10 nM. Additionally, or alternatively, the soluble LAG-3-Ig fusion protein, e.g. hLAG-3 Ig molecule, binds MHC class II positive cells such as DAUDI B cells at 37° C. with a KD of 100 nM and more preferably below 10 nM as defined by Scatchard analysis;
(ii) It induces phenotypic maturation of human monocytes or dendritic cells. Maturation can be monitored by an increase in the expression of cell surface markers such as CD40, CD80 and CD86, and the induction of CD83 expression. Alternatively, activity on myeloid cells can be measured by increased production of cytokines such as TNFα or CCL4;
(iii) It modulates (e.g. stimulates, enhances, or restores) an immune response. The ability of the soluble hLAG-3 Ig molecule to modulate an immune response can be indicated by one or more of: enhancing activation and/or proliferation and/or survival of T and/or NK cells; enhancing cytokine (e g. IL-2, IFN-γ, TNF-α) secretion by T and/or NK cells; enhancing cytotoxic activity of T and/or NK cells against a target cell (e.g., in an in vitro assay).

In one embodiment, the LAG-3 inhibitor for use in the combination therapy is a recombinant LAG-3 protein or related Ig fusion protein. In some embodiments, the LAG-3 inhibitor is chosen from Eftilagimod alpha/IMP321 (Immutep). Eftilagimod alpha is a recombinant soluble version of LAG-3 consisting of the four extracellular domains of LAG-3 fused to the Fc region of an IgG1 (LAG-3-Ig). In some embodiments, the recombinant LAG-3 protein or related Ig fusion protein comprises the polypeptide sequence of SEQ ID NO:30 or their functional variants having at least 90%, 95%, or at least 98% identity to SEQ ID NO:30. Other recombinant LAG-3 proteins or related Ig fusion proteins are disclosed in WO2009/044273 which discloses recombinant LAG-3 proteins and corresponding fusion proteins (which content is incorporated herein by reference in its entirety).

### Exemplary LAG-3 Inhibitors

The following Table 4 discloses specific exemplary LAG-3 inhibitors which may be used in the combination therapy with BTN3A activating antibodies, e.g. ICT01.

TABLE 4

| LAG-3 inhibitors | Company |
|---|---|
| Eftilagimod alpha (IMP321) | Immutep |
| Leramilimab (LAG525, IMP701) | Novartis/Prima Biomed |
| Favezelimab (MK-4280) | Merck Sharp & Dohme |
| Fianlimab (REGN3767) | Regeneron Pharmaceuticals |
| Relatlimab (BMS-986016) | Bristol-Myers Squibb |
| BI 754111 | Boehringer Ingelheim |
| TSR-033 | Tesaro |
| INCAGN2385 | Incyte |
| Sym022 | Symphogen |
| FS118 | F-star Therapeutics |
| Tebotelimab (MGD013) | MacroGenics |
| XmAb22841 | Xencor |

### TIM-3 Inhibitors

In certain embodiments, the combination therapy comprises administering a therapeutically effective amount of a BTN3A activating antibody as described above, e.g., ICT01, in combination with a therapeutically effective amount of a TIM-3 inhibitor.

TIM-3 inhibitors which may be used in the combination therapy as disclosed herein include without limitation anti-TIM-3 antibodies.

In some embodiments, the anti-TIM-3 antibody molecule is a monospecific antibody molecule or a bispecific antibody molecule which exhibits one or more of the following properties
(i) it specifically binds to TIM-3, e.g. human TIM-3. The binding specificity of Ab can be determined in flow cytometry assays by monitoring binding of the antibody to cells expressing TIM-3 protein (e.g. transfected cells or primary immune cells), preferably with an EC50 below 100 nM and more preferably below 10 nM. Additionally, or alternatively, the antibody binds to a recombinant human TIM-3 protein with high affinity, with a K_{D} of 100 nM or less, 10 nM or less, 1 nM or less or 100 μM or less, as measured as measured by SPR, BLI or other suitable binding assays;
(ii) it inhibits binding of TIM-3 to a TIM-3 ligand, e.g., phosphatidylserine (PtdSer), HMGB1, or CEACAM-1 or Galectin-9 with an IC50 below 100 nM and more preferably below 10 nM; and/or,
(iii) it modulates (e.g. stimulates, enhances, or restores) an immune response. The ability of the antibody to modulate an immune response can be indicated by one or more of: enhancing activation and/or proliferation and/or survival of T cells; enhancing cytokine (e g. IL-2, IFN-γ) secretion by T cells; enhancing cytotoxic activity of T and/or NK cells against a target cell (e.g., in an in vitro assay); enhancing capacity of macrophages or antigen presenting cells to stimulate T cell response or delaying tumor growth (e.g. in an in vivo model), and
(iv) optionally, it binds to a non-human primate TIM-3, e.g., cynomolgus TIM-3.

In some embodiments, the TIM-3 inhibitor comprises an anti-TIM-3 antibody molecule. In some embodiments, the TIM-3 inhibitor comprises Sabatolimab (MBG453), TSR-022, LY3321367, Sym023, BGB-A425, INCAGN02390, BMS-986258, RO7121661, BC-3402, SHR-1702, or LY-3415244. In some embodiments, the TIM-3 inhibitor comprises Sabatolimab.

In some embodiments, the anti-TIM-3 antibody molecule comprises the variable heavy chain VH of SEQ ID NO:79 and the variable light chain VL of SEQ ID NO:80 or their functional variants having at least 90%, 95%, or at least 98% identity to SEQ ID NO:79 and SEQ ID NO: 80 respectively. In some embodiments, the anti-TIM-3 antibody molecule comprises the 6 CDRs (HCDR1, HCDR2, HCDR3, LCDR1, LCDR2, LCDR3) of SEQ ID NO: 81-86 respectively. In some embodiments, the anti-TIM-3 antibody molecule comprises the heavy chain of SEQ ID NO: 77 and the light chain of SEQ ID NO:78 or their functional variants having at least 90%, 95%, or at least 98% identity to SEQ ID NO:77 and SEQ ID NO:78 respectively. Other TIM-3 antibody molecules are disclosed in WO2021123902 (which content is incorporated herein by reference in its entirety).

In other embodiments, the anti-TIM-3 antibody molecule is a bispecific molecule with TIM-3 and PD-1 binding specificities. In more specific embodiments, said TIM-3/PD-1 bispecific antibody comprises the VH polypeptides of SEQ ID NO:91 and 93 respectively, and VL polypeptides of SEQ ID NO:92 and 94 respectively, or their functional variants having at least 90%, 95%, or at least 98% identity to SEQ ID NO:91-94 respectively.

In more specific embodiments, said TIM-3/PD-1 bispecific antibody comprises the polypeptides of SEQ ID NO:87-90 or their functional variants having at least 90%, 95%, or at least 98% identity to SEQ ID NO:87-90 respectively. Typically, said TIM-3/PD-1 bispecific antibody for use in the combination therapy is RO7121661 or other related bispecific antibodies as disclosed in WO2017055404 (which content is incorporated herein by reference in its entirety).

### Exemplary TIM-3 Inhibitors

The following Table 5 discloses specific exemplary TIM-3 inhibitors which may be used in the combination therapy with BTN3A activating antibodies, e.g. ICT01.

TABLE 5

| TIM-3 inhibitors | Company |
|---|---|
| Sabatolimab (MBG453) | Novartis Pharmaceuticals |
| TSR-022 | Tesaro |
| SHR-1702 | Jiangsu HengRui Medicine |
| LY3321367 | Eli Lilly and Company |
| BMS-986258 | Bristol Myers Squibb |
| ICAGN02390 | Incyte Corporation |
| Sym023 | Symphogen AS |
| BGBA425 | Beigene |
| RO7121661 | Hoffmann-La Roche |

### TIGIT Inhibitors

In certain embodiments, the combination therapy comprises administering a therapeutically effective amount of a BTN3A activating antibody as described above, e.g., ICT01, in combination with a therapeutically effective amount of a TIGIT inhibitor.

TIGIT inhibitors which may be used in the combination therapy as disclosed herein include without limitation anti-TIGIT antibodies.

In some embodiments, the anti-TIGIT antibody molecule is a monospecific antibody molecule or a bispecific antibody molecule which exhibits one or more of the following properties
(i) it specifically binds to TIGIT, e.g. human TIGIT. The binding specificity of Ab can be determined in flow cytometry assays by monitoring binding of the antibody to cells expressing TIGIT protein (e.g. transfected cells or primary immune cells), preferably with an EC50 below 100 nM and more preferably below 10 nM. Additionally, or alternatively, the antibody binds to a recombinant human TIGIT protein with high affinity, with a K_{D} of 100 nM or less, 10 nM or less, 1 nM or less or 100 μM or less, as measured as measured by SPR, BLI or other suitable binding assays; and/or,
(ii) it inhibits or blocks interaction of TIGIT with a TIGIT ligand, e.g., PVR (CD155) or CD112/PVRL2 or CD113/PVRL3 or inhibits intracellular signaling mediated by TIGIT binding to PVR with an IC50 below 100 nM and more preferably below 10 nM;
(iii) modulates (e.g. stimulates, enhances, or restores) an immune response. The ability of the antibody to modulate an immune response can be indicated by one or more of: enhancing activation and/or proliferation of T cells, enhancing cytokine (e g. IL-2, IFN-g) secretion by T cells; enhancing cytotoxic activity of T and/or NK cells against a target cell (e.g., in an in vitro assay); reducing suppressor activity of Tregs or delaying tumor growth (e.g. in an in vivo model); and,
(iv) optionally, it binds to a non-human primate TIGIT, e.g., cynomolgus TIGIT;

In some embodiments, the TIGIT inhibitor comprises an anti-TIGIT antibody molecule. In some embodiments, the TIGIT inhibitor comprises BMS-986207, Tiragolumab, MK-7684, AB154, COM902, IBI939, BGB-A1217, ASP8374, M6223, HLX301 (TIGIT×PDL1 bispecific), AZD2936 (anti-TIGIT/anti-PD-1 bispecific).

In some embodiments, the TIGIT inhibitor comprises Tiragolumab.

In some embodiments, the anti-TIGIT antibody molecule comprises the variable heavy chain VH of SEQ ID NO:97 and the variable light chain VL of SEQ ID NO:98 or their functional variants having at least 90%, 95%, or at least 98% identity to SEQ ID NO:97 and SEQ ID NO: 98 respectively. In some embodiments, the anti-TIGIT antibody molecule comprises the 6 CDRs (HCDR1, HCDR2, HCDR3, LCDR1, LCDR2, LCDR3) of SEQ ID NO: 99-104 respectively. In some embodiments, the anti-TIGIT antibody molecule comprises the heavy chain of SEQ ID NO: 95 and the light chain of SEQ ID NO: 96 or their functional variants having at least 90%, 95%, or at least 98% identity to SEQ ID NO:95 and SEQ ID NO:96 respectively. Other TIGIT antibody molecules are disclosed in WO2017053748 (which content is incorporated herein by reference in its entirety).

In other embodiments, the anti-TIGIT antibody molecule is a bispecific molecule with TIGIT and PD-1 binding specificities. In more specific embodiments, said TIGIT/PD-1 bispecific antibody comprises the VH polypeptides of SEQ ID NO: 109 and 111 respectively, and VL polypeptides of SEQ ID NO:110 and 112 respectively, or their functional variants having at least 90%, 95%, or at least 98% identity to SEQ ID NO:109-112 respectively. In some embodiments, the anti-TIGIT antibody is a bispecific TIGIT/PD-1 molecule comprising a first VH sequence with 3 CDRs of SEQ ID NO:113-115 respectively, a first VL sequence with 3 CDRs of SEQ ID NO:116-118 respectively, a second VH sequence with 3 CDRs of SEQ ID NO: 119-121, and a second VL sequence with 3 CDRs of SEQ ID NO: 122-124.

In more specific embodiments, said TIM-3/PD-1 bispecific antibody comprises the polypeptides of SEQ ID NO: 105-108 or their functional variants having at least 90%, 95%, or at least 98% identity to SEQ ID NO: 105-108 respectively. Typically, said TIM-3/PD-1 bispecific antibody for use in the combination therapy is AZD2936 or other related bispecific antibodies are disclosed in WO2022/229919 (which content is incorporated herein by reference in its entirety).

### Exemplary TIGIT Inhibitors

The following Table 6 discloses specific exemplary TIGIT inhibitors which may be used in the combination therapy with BTN3A activating antibodies, e.g. ICT01.

TABLE 6

| TIGIT inhibitors | Company |
|---|---|
| Tiragolumab | Hoffmann-La Roche |
| (MTIG7192A; RG6058) | |
| BMS-986207 | Bristol Myers Squibb |
| MK-7684 | Merck |
| AB154 | Arcus Biosciences |
| COM902 | Compugen |
| IBI939 | Innovent Biologics |
| BGB-A1217 | BeiGene |
| ASP8374 | Astellas Pharma |
| M6223 | EMD Serono |
| AZD2936 | AstraZeneca |
| HLX301 | Shanghai Henlius Biotech |

### PD-1 Inhibitors

The disclosed combination therapies may also be used further in combination with PD-1 inhibitors, e.g. a combined administration of ICT01, anti-LAG-3 inhibitors and PD-1 inhibitors.

As used herein, the term “PD-1 inhibitor” includes, but is not limited to, PD-1 binding agents, PD-L1 binding agent and PD-L2 binding agents. PD-1 binding agents include antibodies that specifically bind to PD-1. PD-L1 and PD-L2 binding agents include antibodies that specifically bind to PD-L1 and/or PD-L2, as well as soluble PD-1 polypeptides that bind to PD-L1 and/or PD-L2. In some embodiments, PD-1 pathway inhibitor is a PD-1-binding agent, for example an anti-PD-1 antibody. In some embodiments, the PD-1 inhibitor is a PD-L1-binding agent, for example, an anti-PD-L1 antibody. In further embodiments, the PD-L1-binding agent is a soluble PD-1 polypeptide, for example, a PD-1-Fc fusion polypeptide capable of binding to PD-L1. In further embodiments, the PD-L2-binding agent is a soluble PD-1 polypeptide, for example, a PD-1-Fc fusion polypeptide capable of binding to PD-L2.

In specific embodiments, art recognized anti-PD-1 or anti-PD-L1 antibodies can be used.

In some embodiments, the anti-PD-1 antibody molecule is a monospecific antibody molecule or a bispecific antibody molecule which exhibits one or more of the following properties:
(i) it binds to human PD-1 or human PD-L1 with a K_{D} of 10⁻⁷M or less,
(ii) it inhibits the binding of PD-L1 and/or PD-L2 to PD-1
(iii) it does not bind substantially to human CD28, CTLA-4 or ICOS,
(iv) it increases T-cell proliferation in a Mixed Lymphocyte Reaction (MLR) assay, and/or,
(v) it increases interferon-gamma production in a MLR assay.

In some embodiments, the anti-PD-L1 antibody molecule is a monospecific antibody molecule or a bispecific antibody molecule which exhibits one or more of the following properties:
(i) it binds to human PD-L1 with a K_{D} of 10⁻⁷ M or less,
(ii) it inhibits the binding of PD-L1 to PD-1
(iii) it increases T-cell proliferation in a Mixed Lymphocyte Reaction (MLR) assay, and/or,
(iv) it increases interferon-gamma production in a MLR assay.

In specific embodiments, the anti-PD-1 antibody is selected from the group consisting of nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), PDR001 (Novartis; also known as spartalizumab; see WO 2015/112900), MEDI-0680 (AstraZeneca; also known as AMP-514; see WO 2012/145493), cemiplimab (Regeneron; also known as REGN-2810; see WO 2015/112800), JS001 (TAIZHOU JUNSHI PHARMA; see Si-Yang Liu et al., J. Hematol. Oncol. 70: 136 (2017)), BGB-A317 (“Tislelizumab” Beigene; see WO 2015/35606 and US 2015/0079109), INCSHR1210 (Jiangsu Hengrui Medicine; also known as SHR-1210; see WO 2015/085847; Si-Yang Liu et al., ./. Hematol. Oncol. 70: 136 (2017)), TSR-042 (Tesaro Biopharmaceutical; also known as ANB011; see WO2014/179664), GLS-010 (Wuxi/Harbin Gloria Pharmaceuticals; also known as WBP3055; see Si-Yang Liu et al., J. Hematol. Oncol. 70: 136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), IBI308 (Innovent; see WO 2017/024465, WO 2017/025016, WO 2017/132825, and WO 2017/133540), and BCD-100 (Biocad).

In specific embodiments, anti-PD-1 antibody for use in the combination therapy, in particular with anti-LAG-3 inhibitors and ICT01, include without limitation nivolumab (BMS), pembrolizumab (Merck&Co), avelumab (EMD Serono), durvalumab (AstraZeneca), cemiplimab (Regeneron), pidilizumab (Pfizer), dostarlimab (GlaxoSmithKline), or atezolizumab (Roche).

In specific embodiments, anti-PD-1 antibody for use in the combination therapy, in particular with anti-LAG-3 inhibitors and ICT01, is an antibody having a VH of SEQ ID NO:35 and a VL of SEQ ID NO:36, or their functional variants having at least 90%, 95% or 98% identity to SEQ ID NO: 35 and SEQ ID NO:36 respectively. In specific embodiments, anti-PD-1 antibody for use in the combination therapy, in particular with anti-LAG-3 inhibitors and ICT01, is an antibody having HCDR1 of SEQ ID NO:39, HCDR2 of SEQ ID NO:40, HCDR3 of SEQ ID NO: 41, LCDR1 of SEQ ID NO:42, LCDR2 of SEQ ID NO:43, LCDR3 of SEQ ID NO:44,

In other specific embodiments, anti-PD-1 antibody for use in the combination therapy, in particular in bispecific molecule with additional binding specificity to LAG-3, TIM-3, or TIGIT includes the 6 CDRs of nivolumab of SEQ ID NO:39-44 respectively (HCDR1, HCDR2, HCDR3, LCDR1, LCDR2, LCDR3 respectively).

### Framework or Fc Engineering

The BTN3A activating antibodies for use in the combination therapy of the disclosure can include modifications made to framework residues within VH and VL, to decrease its immunogenicity.

In some specific embodiments, the antibody of the disclosure is a humanized monoclonal antibody of the parent murine antibody mAb 7.2, including at least the following amino acid mutations in the VH framework regions: V5Q, V11L, K12V, R66K, S74F, I75S, E81Q, S82AR, R82BS, R83T, D85E, T87S, L108S, and at least the following amino acid mutations in the VK framework regions: T5N, V15L, R18T, V19I, K42N, A43I, D70G, F73L, Q100G.

In other specific embodiments, the antibody of the disclosure is a humanized monoclonal antibody of the parent murine antibody mAb 7.2, including at least the following amino acid mutations in the VH framework regions as compared to mAb 7.2: V5Q, V11L, K12V, R66K, S74F, I75S, E81Q, S82AR, R82BS, R83T, D85E, T87S, L108S, and at least the following amino acid mutations in the VK framework regions: T5N, V15L, R18T, V19I, K42N, A43I, S63T, D70G, F73L, Q100G.

In addition to modifications made within the framework regions, the antibodies of the disclosure may be engineered to include modifications within the Fc region, typically to alter one or more functional properties of the antibody, such as serum half-life, complement fixation, Fc receptor binding, and/or antigen-dependent cellular cytotoxicity.

Furthermore, an antibody for use in the combination therapy of the disclosure may be chemically modified (e.g., one or more chemical moieties can be attached to the antibody) or be modified to alter its glycosylation, again to alter one or more functional properties of the antibody. Each of these embodiments is described in further detail below.

As used herein, the term “isotype constant region” or “Fc region” is used interchangeably to define the C-terminal region of an immunoglobulin heavy chain, including native sequence Fc region and variant Fc regions. The human IgG heavy chain Fc region is generally defined as comprising the amino acid residue from position C226 or from P230 to the carboxyl-terminus of the IgG antibody wherein the numbering is according to the EU numbering system. The C-terminal lysine (residue K447) of the Fc region may be removed, for example, during production or purification of the antibody or its corresponding codon deleted in the recombinant constructs. Accordingly, a composition of antibodies of the disclosure may comprise antibody populations with all K447 residues removed, antibody populations with no K447 residues removed, and antibody populations having a mixture of antibodies with and without the K447 residue.

In some specific embodiments, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In other embodiments, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In yet other embodiments, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another embodiment, one or more amino acids selected from amino acid residues can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another embodiment, one or more amino acid residues are altered to thereby alter the ability of the antibody to fix complement. This approach is described further in PCT Publication WO 94/29351 by Bodmer et al.

In other embodiments, the Fc region is modified to decrease the ability of the antibody to mediate antibody dependent cellular cytotoxicity (ADCC) and/or to decrease the affinity of the antibody for an Fcγ receptor by modifying one or more amino acids. Such antibodies with decreased effector functions, and in particular decreased ADCC include silent antibodies.

In certain embodiments, the Fc domain of the IgG1 isotype is used. In some specific embodiments, a mutant variant of the IgG1 Fc fragment is used, e.g. a silent IgG1 Fc which reduces or eliminates the ability of the fusion polypeptide to mediate antibody dependent cellular cytotoxicity (ADCC) and/or to bind to an Fcγ receptor.

In certain embodiments, the Fc domain of the IgG4 isotype is used. In some specific embodiments, a mutant variant of the IgG4 Fc fragment is used, e.g. a silent IgG4 Fc which reduces or eliminates the ability of the fusion polypeptide to mediate antibody dependent cellular cytotoxicity (ADCC) and/or to bind to an Fcγ receptor.

Silenced effector functions can be obtained by mutation in the Fc constant part of the antibodies and have been described in the Art (Baudino L et al., J Immunol. 2008 Sep. 15; 181(6):4107-12; Strohl W R. Curr Opin Biotechnol. 2009 December; 20(6):685-91). Examples of silent IgG1 antibodies comprise the triple mutant variant IgG1 L247F L248E P350S. Examples of silent IgG4 antibodies comprise the double mutant variant IgG4 S241P L248E.

In certain embodiments, the Fc domain is a silent Fc mutant preventing glycosylation at position 314 of the Fc domain. For example, the Fc domain contains an amino acid substitution of asparagine at position 314. An example of such amino acid substitution is the replacement of N314 by a glycine or an alanine.

In still other embodiments, the glycosylation of an antibody is modified. For example, an aglycosylated antibody can be made (i.e., the antibody lacks glycosylation). Glycosylation can be altered to, for example, increase the affinity of the antibody for the antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Another modification of the antibodies herein that is contemplated by the present disclosure is pegylation or hesylation or related technologies. An antibody can be pegylated to, for example, increase the biological (e.g., serum) half-life of the antibody. To pegylate an antibody, the antibody, or fragment thereof, typically is reacting with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the disclosure. See for example, EP 0 154 316 by Nishimura et al. and EP 0 401 384 by Ishikawa et al.

Another possibility is a fusion of at least the antigen-binding region of the antibody of the disclosure to proteins capable of binding to serum proteins, such human serum albumin to increase half-life of the resulting molecule. Such approach is for example described in Nygren et al., EP 0 486 525.

In certain embodiments, the C-terminal lysine commonly present on human IgG heavy chain constant domains, is engineered out to reduce heterogeneity due to the cleavage of this reduce commonly observed during manufacturing or storage. Such modifications do not perceptible change the desirable functions of these antibodies, while conferring the benefit of stability to these molecules.

### Nucleic Acid Molecules Encoding Antibodies of the Disclosure

Also disclosed herein are the nucleic acid molecules that encode the BTN3A activating antibodies for use of the present disclosure. Examples of variable light chain and heavy chain nucleotide sequences are those encoding the variable light chain and heavy chain amino acid sequences of any one of mAb1 (ICT01), mAb2, mAb4 and mAb5, the latter sequences being easily derived from the Table 1 and Table 2, and using the genetic code and, optionally taking into account the codon bias depending on the host cell species.

The nucleic acid molecules that derive from the latter sequences having been optimized for protein expression in mammalian cells, for example, CHO cell lines.

The nucleic acids may be present in whole cells, in a cell lysate, or may be nucleic acids in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art (Ausubel et al., 1988, Current Protocols in Molecular Biology (John Wiley & Sons). A nucleic acid of the disclosure can be, for example, DNA or RNA and may or may not contain intronic sequences. In an embodiment, the nucleic acid may be present in a vector such as a phage display vector, or in a recombinant plasmid vector.

Nucleic acids of the disclosure can be obtained using standard molecular biology techniques. Once DNA fragments encoding, for example, VH and VL segments are obtained, these DNA fragments can be further manipulated by standard recombinant DNA techniques, for example to convert the variable region genes to full-length antibody chain genes, to Fab fragment genes or to an scFv gene. In these manipulations, a VL- or VH-encoding DNA fragment (for example VL and VH as defined in Table 1) is operatively linked to another DNA molecule, or to a fragment encoding another protein, such as an antibody constant region or a flexible linker. The term “operatively linked”, as used in this context, is intended to mean that the two DNA fragments are joined in a functional manner, for example, such that the amino acid sequences encoded by the two DNA fragments remain in-frame, or such that the protein is expressed under control of a desired promoter.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (Kabat et al., K. S. (1992). Sequences of Proteins of Immunological Interest (DIANE Publishing)) and DNA fragments encompassing these regions can be obtained by standard PCR amplification. The heavy chain constant region can be an IgG1, IgG2, IgG3, IgG4, IgA, IgE, IgM or IgD constant region. In some embodiments, the heavy chain constant region is selected among IgG1 isotypes, for example human IgG1 isotype. In other embodiments, the heavy chain constant region is selected among IgG4 isotypes, for example human IgG4 isotype. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as to a Fab light chain gene) by operatively linking the VL-encoding DNA to another DNA molecule encoding the light chain constant region, CL. The sequences of human light chain constant region genes are known in the art (Kabat et al., 1992, see supra) and DNA fragments encompassing these regions can be obtained by standard PCR amplification. The light chain constant region can be a kappa or a lambda constant region.

To create an scFv gene, the VH- and VL-encoding DNA fragments are operatively linked to another fragment encoding a flexible linker, e.g., encoding the amino acid sequence (Gly4-Ser)₃, such that the VH and VL sequences can be expressed as a contiguous single-chain protein, with the VL and VH regions joined by the flexible linker (Bird et al., 1988 see supra; Huston et al., 1988, see supra; McCafferty et al., 1990, McCafferty, J., et al, 1990. Nature 348, 552-554).

### Generation of Transfectomas Producing Monoclonal Antibodies

Antibodies of the present disclosure can be produced in a host cell transfectoma using, for example, a combination of recombinant DNA techniques and gene transfection methods as is well known in the art (Morrison, 1985; Science 229, 1202-1207).

For example, to express the antibodies, or antibody fragments thereof, DNAs encoding partial or full-length light and heavy chains can be obtained by standard molecular biology or biochemistry techniques (e.g., DNA chemical synthesis, PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the VH segment is operatively linked to the CH segment(s) within the vector and the VL segment is operatively linked to the CL segment within the vector. Additionally, or alternatively, the recombinant expression vector can encode a signal peptide that facilitates secretion of the antibody chain from a host cell. The antibody chain gene can be cloned into the vector such that the signal peptide is linked in frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors disclosed herein carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel's publication (Goeddel, D. V. (1990). [1] Systems for heterologous gene expression. In Methods in Enzymology, (Academic Press), pp. 3-7). It will be appreciated by those skilled in the art that the design of the expression vector, including the selection of regulatory sequences, may depend on such factors as the choice of the host cell to be transformed, the level of expression of protein desired, etc. Regulatory sequences for mammalian host cell expression include viral elements that direct high levels of protein expression in mammalian cells, such as promoters and/or enhancers derived from cytomegalovirus (CMV), Simian Virus 40 (SV40), adenovirus (e.g., the adenovirus major late promoter (AdMLP)), and polyoma. Alternatively, nonviral regulatory sequences may be used, such as the ubiquitin promoter or P-globin promoter. Still further, regulatory elements composed of sequences from different sources, such as the SRa promoter system, which contains sequences from the SV40 early promoter and the long terminal repeat of human T cell leukemia virus type 1 (Takebe et al., 1988, Mol. Cell. Biol. 8, 466-472).

In addition to the antibody chain genes and regulatory sequences, the recombinant expression vectors of the present disclosure may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. It is theoretically possible to express the antibodies of the present disclosure in either prokaryotic or eukaryotic host cells. Expression of antibodies in eukaryotic cells, for example mammalian host cells, yeast or filamentous fungi, is discussed because such eukaryotic cells, and in particular mammalian cells, are more likely than prokaryotic cells to assemble and secrete a properly folded and immunologically active antibody.

In one specific embodiment, a cloning or expression vector according to the disclosure comprises one of the coding sequences of the heavy and light chains of any one of mAb1, mAb2, mAb4 and mAb5 operatively linked to suitable promoter sequences.

Mammalian host cells for expressing the recombinant antibodies of the disclosure include Chinese Hamster Ovary (CHO cells) including dhfr-CHO cells (described in Urlaub and Chasin, 1980) used with a DHFR selectable marker (as described in Kaufman and Sharp, 1982), CHOK1 dhfr+ cell lines, NSO myeloma cells, COS cells and SP2 cells, for example GS CHO cell lines together with GS Xceed™ gene expression system (Lonza). When recombinant expression vectors encoding antibody genes are introduced into mammalian host cells, the antibodies are produced by culturing the host cells for a period of time sufficient for expression of the antibody in the host cells and, optionally, secretion of the antibody into the culture medium in which the host cells are grown. Antibodies can be recovered and purified for example from the culture medium after their secretion using standard protein purification methods (Shukla et al., 2007, J. Chromatogr. B 848, 28-39).

In one specific embodiment, the host cell of the disclosure is a host cell transfected with an expression vector having the coding sequences suitable for the expression of the heavy and light chains of the antibodies or recombinant proteins for use as disclosed herein, operatively linked to suitable promoter sequences.

For example, the present disclosure relates to a host cell comprising at least the nucleic acids of SEQ ID NO:8 and SEQ ID NO: 10 encoding respectively the heavy and light chains of ICT01.

The latter host cells may then be further cultured under suitable conditions for the expression and production of an antibody for use as disclosed herein.

Alternatively, cell free expression systems may be used for the production of any of the antibodies or recombinant proteins. Typically, methods of cell-free expression of proteins or antibodies are already described (Stech et al., 2017, Sci. Rep. 7, 12030).

### Combination Kits and Compositions

### Combination Kits

The combination of the present disclosure comprising a BTN3A antibody and an immune checkpoint inhibitor as previously defined, typically anti-LAG-3 antibody, anti-TIM-3 antibody and/or anti-TIGIT antibody, may be presented as a combination kit.

By the term “combination kit” “or kit of parts” as used herein is meant the pharmaceutical composition or compositions that are used to administer, the BTN3A activating antibody and the immune checkpoint inhibitor according to the disclosure.

When both compounds are administered simultaneously, the combination kit can contain, for example, the components, suitably the BTN3A activating antibody and the immune checkpoint inhibitor in separate pharmaceutical compositions. When the components, suitably the BTN3A activating antibody and the immune checkpoint inhibitor are not administered simultaneously, the combination kit will contain the active principles in separate pharmaceutical compositions either in a single package or in separate pharmaceutical compositions in separate packages.

In one aspect there is provided a kit of parts comprising
the BTN3A activating antibody in association with pharmaceutically acceptable excipients, diluents or carrier, typically a composition comprising the BTN3A activating antibody as previously defined; and,
the immune checkpoint inhibitor in association with pharmaceutically acceptable excipients, diluents and/or carriers, typically a composition comprising the immune checkpoint inhibitor as previously defined; typically LAG-3 inhibitor, anti-TIM-3 antibody and/or anti-TIGIT antibody.

In one embodiment of the present disclosure the kit of parts comprises:
the BTN3A activating antibody in association with pharmaceutically acceptable excipients, diluents and/or carriers, typically a composition comprising the BTN3A activating antibody as previously defined; and,
the immune checkpoint inhibitor in association with pharmaceutically acceptable excipients, diluents and/or carriers, typically a composition comprising the immune checkpoint inhibitor as previously defined; typically LAG-3 inhibitor, anti-TIM-3 antibody and/or anti-TIGIT antibody,
   wherein the components are provided in a form which is suitable for sequential, separate and/or simultaneous administration.

In one embodiment the kit of parts comprises:
a first container comprising the BTN3A activating antibody in association with pharmaceutically acceptable excipients, diluents and/or carrier, typically a composition comprising the BTN3A activating antibody as previously defined; and
a second container comprising the immune checkpoint inhibitor in association with pharmaceutically acceptable excipients, diluents and/or carriers, typically a composition comprising the immune checkpoint inhibitor as previously defined; typically anti-LAG-3 antibody, anti-TIM-3 antibody and/or anti-TIGIT antibody, and a container means for containing said first and second containers.

The combination kit can also be provided by instruction, such as dosage and administration instructions. Such dosage and administration instructions can be of the kind that is provided to a doctor, or they can be of the kind that are provided by a doctor, such as instructions to a patient.

### Pharmaceutical Compositions

Pharmaceutical compositions suitable for administration to human patients are typically formulated for parenteral administration, e.g., in a liquid carrier, or suitable for reconstitution into liquid solution or suspension for intravenous administration.

In general, such compositions typically comprise a pharmaceutically acceptable carrier. As used herein, the term “pharmaceutically acceptable” means approved by a government regulatory agency or listed in the U.S. Pharmacopeia or another generally recognized pharmacopeia for use in animals, particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil, glycerol polyethylene glycol ricinoleate, and the like. Water or aqueous solution saline and aqueous dextrose and glycerol solutions may be employed as carriers, particularly for injectable solutions (e.g., comprising an BTN3A activating antibody, an anti-LAG-3 and/or anti-PD-1 antibody).

An antibody for use in the combination therapy can be formulated into a composition as above defined in a neutral or salt form. Liquid compositions for parenteral administration can be formulated for administration by injection or continuous infusion. Routes of administration by injection or infusion include intravenous, intraperitoneal, intramuscular, intrathecal and subcutaneous. In one embodiment, the BTN3A activating, anti-LAG-3 and/or anti-PD-1 antibodies are administered intravenously (e.g., in separate formulations or together (in the same formulation or in separate formulations)).

Suitable formulation for solution for infusion or subcutaneous injection of antibodies have been described in the art and for example are reviewed in Cui et al (Drug Dev Ind Pharm 2017, 43(4): 519-530) (which content is incorporated herein in its entirety).

Compositions Comprising BTN3A Activating Antibody, Anti-LAG-3 and/or Anti-PD-1 Antibody and Dosage Regimen

In some embodiments the BTN3A activating antibody as herein defined, an anti-LAG-3 and/or anti-PD-1 antibody may thus be formulated in a composition, e.g., a pharmaceutical composition as defined above, containing either
one or a combination of BTN3A activating antibodies disclosed herein, for example, one antibody selected from the group consisting of ICT01 (mAb1), mAb2, mAb3, mAb4, mAb5 and mAb6 or their antigen-binding portions, formulated together with a pharmaceutically acceptable carrier;
one or a combination of LAG-3 inhibitors as herein defined, for example, a LAG-3 inhibitor selected from the group consisting of eftilagimod alpha, opdualag (Nivolumab/Relatlimab), tebotelimab; and,
optionally, one or a combination of PD-1 inhibitors as herein defined, for example, an anti-PD-1 antibody selected from the group consisting of nivolumab (BMS), pembrolizumab (Merck&Co), avelumab (EMD Serono), durvalumab (AstraZeneca), cemiplimab (Regeneron), pidilizumab (Pfizer), dostarlimab (GlaxoSmithKline), or atezolizumab (Roche).

In preferred embodiments, the pharmaceutical composition for use in the combination therapy comprises a first pharmaceutical composition with ICT01 formulated for intravenous infusion as above defined, and a second pharmaceutical composition including a LAG-3 inhibitor, preferably selected from eftilagimod alpha, opdualag (Nivolumab/Relatlimab), tebotelimab, also formulated for intravenous administration, and optionally, and third pharmaceutical composition including a PD-1 inhibitor, preferably selected from the group consisting of nivolumab (BMS), pembrolizumab (Merck&Co), avelumab (EMD Serono), durvalumab (AstraZeneca), cemiplimab (Regeneron), pidilizumab (Pfizer), dostarlimab (GlaxoSmithKline), or atezolizumab (Roche), also formulation for intravenous administration.

The pharmaceutical compositions comprising respectively BTN3A activating antibody (e.g ICT01), the LAG-3 inhibitors and optionally the PD-1 inhibitors, can be formulated at various concentrations.

For example, a formulation for use in the combination therapy may comprise the activating BTN3A activating antibody (e.g; ICT01) at a concentration of between 0.1 μM and 1 mM, more preferably between 1 μM and 500 μM, between 500 μM and 1 mM, between 300 μM and 700 μM, between 1 μM and 200 μM, between 100 μM and 200 μM, between 200 M and 300 μM, between 300 μM and 400 μM, between 400 μM and 500 μM, between 500 μM and 600 μM, between 600 μM and 700 μM, between 800 μM and 900 μM or between 900 μM and 1 mM. Typically, the formulation comprises the BTN3A activating antibody at a concentration of between 300 μM and 700 μM.

### Uses and Methods of the Combination of the Disclosure

The present disclosure provides a therapeutic combination (also referred as “combination therapy”) comprising a BTN3A activating antibody, e.g., ICT01, and an immune checkpoint inhibitor, e.g. a LAG-3 inhibitor, anti-TIM-3 antibody or anti-TIGIT antibody (and related bispecific molecules with anti-PD-1 molecule) as previously defined for use in the treatment of cancer, optionally further in combination with a PD-1 inhibitor, e.g. anti-PD-1 or anti-PD-L1 antibody as described in the previous sections.

The present disclosure also provides a method of treatment of cancer in a subject in need thereof comprising administering to said subject a therapeutically effective amount of a BTN3A activating antibody, e.g. ICT01, in combination, simultaneously, sequentially, or separately with a therapeutically effective amount of an immune checkpoint inhibitor selected from LAG-3 inhibitors, TIM-3 inhibitors and TIGIT inhibitors, e.g. anti-LAG-3 antibody, anti-TIM-3 antibody or anti-TIGIT antibody.

As used herein, the term “treat” “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease or reducing or alleviating one or more symptoms of the disease. In particular, with reference to the treatment of a tumor, the term “treatment” may refer to the inhibition of the growth of the tumor, or the reduction of the size of the tumor.

BTN3A activating antibodies can activate the cytolytic function, cytokine production and/or proliferation of Vγ9Vδ2 T cells, and thereby may be used to overcome the immunosuppressive mechanisms observed in cancer patients (see notably WO2012080769, WO2012080351, and WO2020025703). The results of the present disclosure now show that the present combination therapy with additional immune checkpoint inhibitor, such as LAG-3, TIM-3 or TIGIT inhibitors, promotes further synergistic and specific Vγ9Vδ2 T cell activation and cytotoxicity in human PBMCs highlighting its therapeutic interest, notably for the treatment of cancer.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

Examples of cancers include, but are not limited to, hematological malignancies such as myeloid cell lineage neoplasm including acute myeloid leukemia (AML); B-cell lymphoid neoplasm including Hodgkin lymphoma, B cell non-Hodgkin lymphoma (B-NHL), diffuse large B-cell lymphoma (DLBCL), indolent NHL, chronic lymphocytic leukemia (CLL), small lymphocytic lymphoma (SLL), mantle cell lymphoma (MCL), multiple myeloma; T-cell lymphoid neoplasm and NK-cell lymphoid neoplasm

Examples of non-hematological cancers (i.e., solid tumors) include, but are not limited to, advanced tumors, refractory or recurrent tumors, melanoma (e.g. metastatic malignant melanoma), lung cancer (e.g. non-small cell lung cancer), breast cancer, prostate cancer, head and neck cancer (e.g. squamous cell carcinoma of head and neck), renal cancer, colorectal cancer, gastric cancer, esophageal cancer, liver cancer, pancreatic cancer, glioblastoma, ovarian cancer, cervical cancer, cholangiocarcinoma, bladder cancer and cancer of the kidney or ureter and PD-L1 positive cancers.

In the combination therapy of the present disclosure, the BTN3A activating antibody and the immune checkpoint inhibitor, e.g. a LAG-3 inhibitor, anti-TIM-3 antibody or anti-TIGIT antibody, and, optionally, the PD-1 inhibitor, can be independently administered orally or parenterally, including the intravenous, intramuscular, intraperitoneal, subcutaneous, rectal, topical, and transdermal routes of administration.

Sequential administration (e.g., in separate pharmaceutical compositions) is particularly useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

In some embodiments, the BTN3A activating antibody is administered concurrently with the immune checkpoint inhibitor, e.g. a LAG-3 inhibitor, anti-TIM-3 antibody or anti-TIGIT antibody.

In other embodiments, the BTN3A activating antibody is administered before administration of the immune checkpoint inhibitor, e.g. a LAG-3 inhibitor, anti-TIM-3 antibody or anti-TIGIT antibody, while in other embodiments the BTN3A activating antibody is administered after administration of the immune checkpoint inhibitor, e.g. a LAG-3 inhibitor, anti-TIM-3 antibody or anti-TIGIT antibody.

Selecting a dosage regimen (also referred to herein as an administration regimen) for a combination therapy of the disclosure depends on several factors, including the serum or tissue turnover rate of the entity, the level of symptoms, the immunogenicity of the entity, and the accessibility of the target cells, tissue or organ in the individual being treated. Preferably, a dosage regimen maximizes the amount of each therapeutic agent delivered to the patient consistent with an acceptable level of side effects. Accordingly, the dose amount and dosing frequency of each biotherapeutic and chemotherapeutic agent in the combination depends in part on the particular therapeutic agent, the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy.

Any suitable dosage range may be used as determined by attending medical personnel. Dosage regimens can be adjusted to provide the optimum desired response (e.g., a therapeutic or prophylactic response).

In some embodiments, at least one of the therapeutic agents in the combination therapy is administered using the same dosage regimen (dose, frequency and duration of treatment) that is typically employed when the agent is used as monotherapy for treating the same cancer. In other embodiments, the patient receives a lower total amount of at least one of the therapeutic agents in the combination therapy than when the agent is used as monotherapy, e.g., smaller doses, less frequent doses, and/or shorter treatment duration.

Regarding the dosage regimen of the BTN3A activating antibody, e.g., ICT01, and of the immune checkpoint inhibitor, e.g. a LAG-3 inhibitor, anti-TIM-3 antibody or anti-TIGIT antibody, of the present combination therapy, any suitable dosage range may be used as determined by attending medical personnel.

Dosage regimens can be adjusted to provide the optimum desired response (e.g., a therapeutic or prophylactic response). The antibodies of the disclosure may be formulated within a therapeutic mixture to comprise about 0.0001 to 100.0 milligrams, or about 0.001 to 10 milligrams, or about 0.0001 to 1.0 milligrams, or about 0.001 to 0.1 milligrams, or about 0.1 to 1.0 milligrams or even 1.0 to about 10 milligrams per dose. Multiple doses can also be administered. Typically, the therapeutic dose of the BTN3A activating antibody, e.g. ICT01, in a human patient will be in the range of 100 pg to 700 mg per administration (based on a body weight of 70 kg). For example, the maximum therapeutic dose may be in the range of 0.1 to 10 mg/kg per administration, e.g., between 0.1 and 5 mg/kg or between 1 and 5 mg/kg or between 0.1 and 2 mg/kg. It will be appreciated that such a dose may be administered at different intervals, as determined by the oncologist/physician; for example, a dose may be administered daily, twice-weekly, weekly, bi-weekly, every three weeks or monthly.

Typically, the therapeutic dose of the BTN3A activating antibody (e.g; ICT01), LAG-3 inhibitors (e.g. eftilagimod alpha, opdualag (Nivolumab/Relatlimab), or tebotelimab) for use in the combination therapy in a human patient, will be in the range of 100 pg to 700 mg per administration (based on a body weight of 70 kg). For example, the maximum therapeutic dose may be in the range of 0.1 to 10 mg/kg per administration, e.g. between 0.1 and 5 mg/kg or between 1 and 5 mg/kg or between 0.1 and 2 mg/kg. It will be appreciated that such a dose may be administered at different intervals, as determined by the oncologist/physician; for example, a dose may be administered daily, twice-weekly, weekly, bi-weekly, every three weeks or monthly. The skilled person may also select the prescribed dosing regimen for approved monotherapy for LAG-3 inhibitors and/or PD-1 inhibitors.

Typically, in specific embodiments, said activating BTN3A antibody (e.g, ICT01) is administered intravenously at a dose comprised between 20 μg and 200 mg, notably between 1 mg and 200 mg or between 7 and 200 mg each dose, typically every 21 days.

In specific embodiments, said LAG-3 inhibitors, for example, an anti-LAG-3 antibody such as favelezimab, is administered intravenously at a unit dose ranging from 20 μg and 1000 mg, notably between about 100 to about 1000 mg, e.g., 800 mg, for example once every 21 days for 1 to 35 cycles, either concurrently with the BTN3A activating antibody (e.g. ICT01) or sequentially.

In other specific embodiments, said LAG-3 inhibitors, for example, the fixed dose combination Opdualag, is administered intravenously at a unit dose of 160 mg relatlimab and 480 mg of nivolumab in a single intravenous infusion, for example every 4 weeks, either concurrently with the BTN3A activating antibody (e.g. ICT01) or sequentially.

In specific embodiments, suitable unit dose for intravenous administration of an activating anti-BTN3A antibody can be selected from 1, 7, 10, 20, 50, 75, 100, 125, 150, 175 and 200 mg.

In specific embodiments suitable unit dose for subcutaneous administration of a LAG-3 recombinant protein or Ig fusion can be selected from about 0.25-30 mg (e.g. eftilagimod alpha).

In specific embodiments suitable unit dose for intravenous administration of a PD-1 inhibitor can be selected from 100 to 2000 mg (e.g. preferably selected from the group consisting of nivolumab, pembrolizumab, avelumab, durvalumab, cemiplimab, pidilizumab, dostarlimab, or atezolizumab, also formulation for intravenous administration.

In specific embodiments, a BTN3A activating antibody (preferably ICT01 (mAb1) as described herein) is administered in the present combination therapy (typically with LAG-3 inhibitors and optionally anti-PD-1 antibodies) at a unit dose of about 7 to about 200 mg, e.g., 75 mg, for example once every 21 days, for 1 to 22 cycles.

Advantageously, in specific embodiments, the combined effect of the BTN3A activating antibody (e.g., ICT01) treatment and the immune checkpoint inhibitors (e.g., LAG-3 inhibitor, TIM-3 inhibitor, or TIGIT inhibitor, and optionally the PD-1 inhibitor), increases the overall survival in subjects to at least 10%, 20%, 30%, 40%, or at least 50% as compared to monotherapy with either said BTN3A antibody, or immune checkpoint inhibitors (such as LAG-3 inhibitors, TIM-3 or TIGIT inhibitors.

“Overall survival” (OS) is defined herein as the time from date of first dose to date of death due to any cause in participants of a clinical study.

In specific embodiments, the combined effect of the BTN3A activating antibody (e.g., ICT01) treatment and the immune checkpoint inhibitors (e.g., LAG-3 inhibitor, TIM-3 inhibitor, or TIGIT inhibitor, and optionally the PD-1 inhibitor) increases the progression-free survival to at least 10%, 20%, 30%, 40%, or at least 50% as compared to monotherapy with either said BTN3A antibody, or immune checkpoint inhibitors (such as LAG-3 inhibitors, TIM-3 or TIGIT inhibitors).

The term “Progression-free survival” (PFS), as used herein, is defined as the time from the date of first dose to the date of confirmed progression.

In certain aspects, the combined therapy can inhibit, delay, and/or reduce tumor growth in the subject. In certain aspects, the growth of the tumor is delayed by at least 50%, 60%, 70% or 80% in comparison to an untreated control subject. In certain aspects, the growth of the tumor is delayed by at least 80% in comparison to an untreated control subject. In certain aspects, the growth of the tumor is delayed by at least 50%, 60%, 70% or 80% in comparison to the predicted growth of the tumor without the treatment. In certain aspects, the growth of the tumor is delayed by at least 50%, 60%, 70% or 80% in comparison to the predicted growth of the tumor without the treatment or with the corresponding monotherapy with BTN3A activating antibody or immune checkpoint inhibitor treatment (such as LAG-3, TIM-3 or TIGIT inhibitor). Assessment of the volume of the tumor may be determined by using RECIST for solid tumor patients (Eisenhauer E A et al., Eur J Cancer. 2009 January; 45(2):228-47.) and RECIL for lymphoma patients (Younes A et al., Ann Oncol. 2017 Jul. 1; 28(7):1436-1447.).

In certain aspects, the administration of combined therapy of the present disclosure can increase the length of survival of the subject. In certain aspects, the increase in survival is in comparison to an untreated control subject or control subject with standard of care treatment. In certain aspects, the increase in survival is in comparison to the predicted length of survival of the subject with the standard of care treatment. In certain aspects, the length of survival is increased by at least 3 times, 4 times, or 5 times the length in comparison to an untreated control subject or a control subject with standard of care treatment. In certain aspects, the length of survival is increased by at least one week, two weeks, one month, two months, three months, six months, one year, two years, or three years in comparison to control subject with standard of care treatment.

The combination therapy comprising the BTN3A activating antibody and the immune checkpoint inhibitor, e.g. LAG-3 inhibitors, TIM-3 inhibitors or TIGIT inhibitors, as previously defined, whether in a mixed single composition or in separate compositions, can further be administered in conjunction with other drugs e.g., for the treatment or prevention of diseases mentioned above.

In specific embodiments, the combination therapy as herein disclosed (typically ICT01 as previously described and the immune checkpoint inhibitor, e.g. LAG-3 inhibitors, TIM-3 inhibitors or TIGIT inhibitors), may be administered in combination with anti-neoplastic agents.

In other specific embodiments, the combination therapy (typically ICT01 as previously described and the immune checkpoint inhibitor, e.g. LAG-3 inhibitors, TIM-3 inhibitors or TIGIT inhibitors), as herein disclosed may be administered in combination with cell therapy (in particular γδT cell therapy).

In other specific embodiments, the combination therapy as herein disclosed (typically ICT01 as previously described and the immune checkpoint inhibitor, e.g. LAG-3 inhibitors, TIM-3 inhibitors or TIGIT inhibitors), may be administered with other immune checkpoint inhibitors (in particular, PD-1 inhibitors such as anti-PD-1 or anti-PD-L1 antibody, and anti-CTLA-4 antibody).

In other specific embodiments, the combination therapy as herein disclosed (typically ICT01 as previously described and the immune checkpoint inhibitor, e.g. LAG-3 inhibitors, TIM-3 inhibitors or TIGIT inhibitors), may be further administered with a cytokine (such as interleukin 2 (IL-2) (Choudhry H et al, 2018, Biomed Res Int. 2018 May 6), interleukin 15 (IL-15) (Patidar M et al., Cytokine Growth Factor Rev. 2016 October; 31:49-59), interleukin 21 (IL-21) (Caccamo N. et al., PLOS One. 2012; 7(7):e41940), or interleukin 33 (IL-33) (Duault C et al., J Immunol. 2016 Jan. 1; 196(1):493-502)), or their recombinant forms and their derivatives, or any cytokines or derivatives capable of inducing lymphocyte activity and/or expanding lymphocyte cells (e.g. proliferation or cytokine production or metabolic changes). The term derivative is used in particular for any cytokine modifications that can rely on PEGylation (e.g. conjugation to polyethylene glycol (PEG) chains), mutation such as amino acid deletion, substitution or insertion, or association with potentiating agents (for example IL15/IL15Ra complexes fused to an IgG1 Fc, in which IL-15 is additionally mutated (asn72asp) that further increase biological activity making this complex an IL-2 and IL-15Rβγ superagonist (Rhode P R et al, Cancer Immunol Res. 2016; 4(1):49-60)) (Barroso-Sousa R et al, Curr Oncol Rep. 2018 Nov. 15; 21(1):1).

Examples of suitable IL2 cytokines includes Proleukin.

Suitable doses of IL-2 cytokine, e.g. Proleukin, which may be administered to the subject, e.g. subcutaneously, with the combination therapy of the present disclosure, ranges from 0.5·10⁶ IU to 18·10⁶ IU per m². In specific embodiments, said IL-2 cytokine is daily administered days 1-5 every 21 days cycle of administration of BTN3A activating antibody.

The term “IL-2” has its general meaning and refers to the human interleukin-2. IL-2 is part of the body's natural immune response. IL-2 mainly regulates lymphocyte activity by binding to IL-2 receptors.

The term “IL-15” has its general meaning and refers to the human interleukin-15. Like IL-2, IL-15 binds to and signals through a complex composed of IL-2/IL-15 receptor beta chain (CD122) and the common gamma chain (gamma-C, CD132). IL-15 regulates the activation and proliferation of T and natural killer (NK) cells.

The term “IL-21” has its general meaning and refers to the human interleukin-21. IL-21 has been ascribed to pleiotropic properties, including, but not limited to, enhancing NK cell and CD8+ T cell cytotoxicity, modulating plasma cell differentiation and inhibiting Treg cells.

The term “IL-33” has its general meaning and refers to the human interleukin-33. IL-33, considered as an alarmin released upon tissue stress or damage, is a member of the IL-1 family and binds the ST2 receptor. IL-33 is known as an effective stimulator of TH1 immune cells, natural killer (NK) cells, iNKT cells, and CD8 T lymphocytes.

In other specific embodiments, the combination therapy as herein disclosed (typically ICT01 as previously described and the immune checkpoint inhibitor, e.g. anti-LAG-3 antibody, anti-TIM-3 antibody or anti-TIGIT antibody) may be administered with further immunotherapeutic drugs, such as immune checkpoint inhibitors (in particular, anti-PD-1, anti-PD-L1, and anti-CTLA-4 antibody).

As used herein, the term “cell therapy” refers to a therapy comprising the in vivo administration of at least a therapeutically effective amount of a cell composition to a subject in need thereof. The cells administered to the patient may be allogenic or autologous. The term “γδ T cell therapy” refers to a cell therapy wherein the cell composition includes, as the active principle, γδ T cells, in particular Vγ9Vδ2 T cells. In specific embodiments, said Vγ9Vδ2 T cells have been expanded and/or activated ex vivo.

A cell therapy product refers to the cell composition which is administered to said patient for therapeutic purposes. Said cell therapy product include a therapeutically effective dose of cells and optionally, additional excipients, adjuvants or other pharmaceutically acceptable carriers.

Another therapeutic strategy is based on the use of the property of the herein disclosed combination as agents which selectively expand and/or activate Vγ9Vδ2 T cells isolated from a sample of a human subject.

The disclosure thus relates to a method for treating a subject in need thereof, comprising:
(a) isolating cells comprising Vγ9Vδ2 T cells, for example PBMCs from a blood sample or tumor-infiltrating leukocytes (TILs) from tumor tissues of a subject,
(b) culturing said isolated cells in an appropriate cell culture medium with an effective amount of the BTN3A activating antibody and of the immune checkpoint inhibitor, e.g. LAG-3 inhibitors, TIM-3 inhibitors or TIGIT inhibitors, can be added simultaneously, concurrently or sequentially to the cell culture, with optionally, other tumor or accessory cells, and thereby obtaining expanded Vγ9Vδ2 T cells,
(c) collecting the expanded Vγ9Vδ2 T cells,
(d) optionally, formulating the expanded Vγ9Vδ2 T cells and administering a therapeutically effective amount of said Vγ9Vδ2 T cells to the subject.

The disclosure further relates to the use of the combination disclosed herein to selectively expand Chimeric Antigen Receptor (CAR) Vγ9Vδ2 T cells. CAR γδ T cells and their use in adoptive T cell cancer immunotherapy are described for example in Mirzaei et al (Cancer Lett 2016, 380(2): 413-423).

The disclosure also relates to the combination as herein defined for use in vivo to potentiate tumor cells in a γδ T cell therapy in a subject in need thereof, typically suffering from cancer, wherein the BTN3A activating antibody and the immune checkpoint inhibitor, e.g. LAG-3 inhibitors, TIM-3 inhibitors or TIGIT inhibitors, can be administered simultaneously, concurrently or sequentially to the subject.

As used herein, the term γδ T cell therapy refers to a therapy which comprises the administration to a subject in need thereof of at least an efficient amount of γδ T cells. Such γδ T cells may be allogeneic or autologous. In specific embodiments, the γδ T cells can be genetically engineered by deletion or knock-out or insertion or knock-in of specific genes. In specific embodiments, said γδ T cells include γδ T cells expressing chimeric antigen receptor. The γδ T cells may have been expanded and/or purified ex vivo. Alternatively, the γδ T cells may also be comprised in a cell composition comprising other blood cells, and for example other cells of the immune system. For references regarding γδ T cell therapy, please see Pauza C D. et al, Front Immunol. 2018 Jun. 8; 9:1305. doi: 10.3389, Saudemont A. et al, Front Immunol. 2018 Feb. 5; 9:153. doi: 10.3389.

The disclosure thus relates to a method of treatment of a subject suffering from cancer including solid tumors or hematological malignancies, in particular, leukemias such as acute myeloid leukemia, and having tumor cells, for example blood tumor cells, said method comprising:
i. administering in said subject an efficient amount of BTN3A activating antibodies as disclosed herein, typically ICT01, mAb2, mAb3, mAb4, mAb5 or mAb6 in combination with an efficient amount of the immune checkpoint inhibitor, e.g. LAG-3 inhibitors, TIM-3 inhibitors or TIGIT inhibitors, wherein said BTN3A activating antibody and the immune checkpoint inhibitor, e.g. LAG-3 inhibitors, TIM-3 inhibitors or TIGIT inhibitors, can be administered simultaneously, concurrently or sequentially, and,
ii. administering an efficient amount of γδ T cell composition in said subject,
   wherein the combination of said efficient amount of BTN3A activating antibodies and said efficient amount of the immune checkpoint inhibitor, e.g. LAG-3 inhibitors, TIM-3 inhibitors or TIGIT inhibitors, has the capacity to potentiate antitumor cytolysis mediated by said γδ T cell composition against said tumor cells.

The disclosure further relates to a method for treating a subject suffering from cancer with solid tumor cells, e.g. advanced tumors, refractory or recurrent tumors, melanoma (e.g. metastatic malignant melanoma), lung cancer (e.g. non-small cell lung cancer), breast cancer, prostate cancer, head and neck cancer (e.g. squamous cell carcinoma of head and neck), renal cancer, colorectal cancer, gastric cancer, esophageal cancer, liver cancer, pancreatic cancer, glioblastoma, ovarian cancer, cervical cancer, cholangiocarcinoma, bladder cancer, cancer of the kidney or ureter and PD-L1 positive cancers and hematological malignancies including for example acute myeloid leukemia (AML), Hodgkin's lymphoma, multiple myeloma, diffuse large B-cell lymphoma (DLBCL), indolent non-Hodgkin lymphoma (NHL), said method comprising:
i. administering in said subject a therapeutically effective amount of BTN3A activating antibodies as disclosed herein, typically ICT01, in combination with a therapeutically effective amount of the immune checkpoint inhibitor, e.g. anti-LAG-3 antibody, anti-TIM-3 antibody or anti-TIGIT antibody, as herein defined, wherein said BTN3A activating antibody and the immune checkpoint inhibitor, e.g. anti-LAG-3 antibody, anti-TIM-3 antibody or anti-TIGIT antibody, can be administered simultaneously, concurrently or sequentially, and,
ii. administering an efficient amount of γδ T cell composition in said subject,
   wherein the combination of said therapeutically effective amount of BTN3A activating antibodies with said therapeutically effective amount of the immune checkpoint inhibitor, e.g. LAG-3 inhibitors, TIM-3 inhibitors or TIGIT inhibitors, has the capacity to potentiate antitumor cytolysis mediated by said γδ T cell composition against said tumor cells.

### Specific Embodiments

E1. A BTN3A activating antibody, for use in the treatment of cancer in a subject in need thereof, wherein a therapeutically effective amount of said BTN3A activating antibody is administered to said subject, in combination with a therapeutically effective amount of an immune checkpoint inhibitor, wherein said immune checkpoint inhibitor is selected from the group consisting of LAG-3 inhibitors, TIGIT inhibitors and TIM-3 inhibitors, and, optionally further in combination with PD-1 inhibitors.

E2. The BTN3A activating antibody for use according to E1, wherein said BTN3A antibody binds to human BTN3A with a K_{D} of 10 nM or lower, preferably with a K_{D} of 5 nM or lower, as measured by surface plasmon resonance.

E3. The BTN3A activating antibody for use according to any one of E1 or E2, wherein said BTN3A activating antibody induces the activation of γδ T cells, typically Vγ9Vδ2 T cells, in co-culture with BTN3A expressing cells, with an EC₅₀ below 5 μg/ml, preferably of 1 μg/ml or below, as measured in a degranulation assay.

E4. The BTN3A activating antibody for use according to any one of E1-E3, wherein said BTN3A activating antibody, either:
comprises (a) a variable heavy chain (VH) polypeptide comprising an amino acid sequence that is at least about 95%, 96%, 97%, 98%, 99% or 100% identical to the SEQ ID NO: 1, and (b) a variable light chain (VL) polypeptide comprising an amino acid sequence that is at least about 95%, 96%, 97%, 98%, 99% or 100% identical to SEQ ID NO: 2 or SEQ ID NO: 3;
comprises HCDRs1-3 of SEQ ID NO: 12-14 and LCDRs1-3 of SEQ ID NO: 15-17;
comprises HCDRs1-3 of SEQ ID NO: 18-20 and LCDRs1-3 of SEQ ID NO:21-23; or,
competes for binding with an antibody selected from mAb 20.1 as produced by the hybridoma deposited at the CNCM under deposit number I-4401, mAb 7.2 as produced by the hybridoma deposited at the CNCM under deposit number I-4402, and/or an antibody having a heavy chain of SEQ ID NO:4 and a light chain of SEQ ID NO:6.

E5. The BTN3A activating antibody for use according to any one of E1-E4, wherein said BTN3A activating antibody comprises HCDRs1-3 of SEQ ID NO: 12-14 and LCDRs1-3 of SEQ ID NO: 15-17.

E6. The BTN3A activating antibody for use according to any one of E1-E5, wherein said BTN3A activating antibody comprises (a) a variable heavy chain (VH) polypeptide comprising an amino acid sequence that is at least about 95%, 96%, 97%, 98%, 99% or 100% identical to the amino acid sequence of SEQ ID NO: 1, and (b) a variable light chain (VL) polypeptide comprising an amino acid sequence that is at least about 95%, 96%, 97%, 98%, 99% or 100% identical to of SEQ ID NO: 2.

E7. The BTN3A activating antibody for use according to any one E1-E6, wherein said BTN3A activating antibody is an antibody comprising or essentially consisting of a heavy chain of SEQ ID NO: 4 and a light chain of SEQ ID NO:6.

E8. The BTN3A activating antibody for use according to any one of E1-E7, wherein said BTN3A activating antibody comprises a mutant or chemically modified IgG1 constant region, wherein said mutant or chemically modified IgG1 constant region confers no or decreased binding to Fcγ receptors when compared to a corresponding antibody with wild type IgG1 isotype constant region.

E9. The BTN3A activating antibody for use according to any one of E1-E8, wherein said mutant IgG1 constant region is the IgG1 triple mutant L247F L248E and P350S.

E10. The BTN3A activating antibody for use according to any one of E1-E9, wherein said immune checkpoint inhibitor is a LAG-3 inhibitor, preferably a monospecific antibody or bispecific antibody, or a fixed dose combination comprising an anti-LAG-3 antibody which either,
comprises (a) a variable heavy chain (VH) polypeptide comprising an amino acid sequence that is at least about 95%, 96%, 97%, 98%, 99% or 100% identical to the SEQ ID NO: 37, and (b) a variable light chain (VL) polypeptide comprising an amino acid sequence that is at least about 95%, 96%, 97%, 98%, 99% or 100% identical to SEQ ID NO: 38;
comprises HCDRs1-3 of SEQ ID NO:45-47 and LCDRs1-3 of SEQ ID NO:48, 43 and 49 or,
competes for binding to LAG-3 with an anti-LAG-3 antibody selected from relatlimab and favezelimab.

E11. The BTN3A activating antibody for use according to any one of E1-E10, wherein the BTN3A activating antibody is administered at a unit dose ranging from about 1 to about 200 mg.

E12. The BTN3A activating antibody for use according to any one of E1-E11, wherein said immune checkpoint inhibitor is a LAG-3 inhibitor, for example, an anti-LAG-3 antibody administered at a unit dose ranging from 100 to 1000 mg.

E13. The BTN3A activating antibody for use according to any one of E1-E11, wherein said immune checkpoint inhibitor is a recombinant LAG-3 protein, for example a LAG-3-Ig fusion protein.

E14. The BTN3A activating antibody for use according to any one of E1-E13, wherein said cancer is selected from the group consisting of melanoma (e.g. metastatic malignant melanoma), lung cancer (e.g. non-small cell lung cancer), breast cancer, prostate cancer, head and neck cancer (e.g. squamous cell carcinoma of head and neck), renal cancer, colorectal cancer, gastric cancer, esophageal cancer, liver cancer, pancreatic cancer, glioblastoma, ovarian cancer, cervical cancer, cholangiocarcinoma, bladder cancer, cancer of the kidney or ureter, PD-L1 positive cancers and hematological malignancies including for example, acute myeloid leukemia, Hodgkin's lymphoma, multiple myeloma, diffuse large B-cell lymphoma (DLBCL), and indolent non-Hodgkin lymphoma (NHL).

E15. The BTN3A activating antibody for use according to any of E1-E14, wherein said subject is refractory or relapse to PD-1 inhibitor therapy, for example said subject is refractory to Ipilimumab and/or nivolumab therapy.

E16. The BTN3A activating antibody for use according to any one of E1-E15, wherein said BTN3A activating antibody is administered once every three weeks, or once every four weeks.

E17. The BTN3A activating antibody for use according to any one of E1-E16, wherein the BTN3A activating antibody is administered intravenously, for example, at a unit dose of about 1 to about 200 mg, e.g., 75 mg, for example once every 21 days for 1 to 22 cycles.

E18. The BTN3A activating antibody for use according to any one of E1-E17, wherein said immune checkpoint inhibitor is an anti-LAG-3/anti-PD-1 bispecific antibody which is administered intravenously at a unit dose of about 100 to about 1000 mg, e.g., 600 mg, for example once every 21 days for 1 to 35 cycles.

E19. The BTN3A activating antibody for use according to any one of E1-E18, wherein said BTN3A antibody comprises a heavy chain of SEQ ID NO:4 and a light chain of SEQ ID NO:6, and said immune checkpoint inhibitor is nivolumab/relatlimab.

E20. A method of treating cancer in a subject in need thereof, said method comprising administering a therapeutically effective amount of a BTN3A activating antibody in combination with a therapeutically effective amount of an immune checkpoint inhibitor, wherein said immune checkpoint inhibitor is selected from the group consisting of LAG-3 inhibitors, TIGIT inhibitors and TIM-3 inhibitors, and, optionally further in combination with PD-1 inhibitors.

E21. The method of E19, wherein said BTN3A antibody binds to human BTN3A with a K_{D} of 10 nM or lower, preferably with a K_{D} of 5 nM or lower, as measured by surface plasmon resonance.

E22. The method of E20 or E21, wherein said BTN3A activating antibody induces the activation of γδ T cells, typically Vγ9Vδ2 T cells, in co-culture with BTN3A expressing cells, with an EC₅₀ below 5 μg/ml, preferably of 1 μg/ml or below, as measured in a degranulation assay.

E23. The method of E20-E22, wherein said BTN3A activating antibody, either:
comprises (a) a variable heavy chain (VH) polypeptide comprising an amino acid sequence that is at least about 95%, 96%, 97%, 98%, 99% or 100% identical to the SEQ ID NO: 1, and (b) a variable light chain (VL) polypeptide comprising an amino acid sequence that is at least about 95%, 96%, 97%, 98%, 99% or 100% identical to SEQ ID NO: 2 or SEQ ID NO: 3;
comprises HCDRs1-3 of SEQ ID NO: 12-14 and LCDRs1-3 of SEQ ID NO: 15-17;
comprises HCDRs1-3 of SEQ ID NO: 18-20 and LCDRs1-3 of SEQ ID NO:21-23; or,
competes for binding with an antibody selected from mAb 20.1 as produced by the hybridoma deposited at the CNCM under deposit number I-4401, mAb 7.2 as produced by the hybridoma deposited at the CNCM under deposit number I-4402, and/or an antibody having a heavy chain of SEQ ID NO:4 and a light chain of SEQ ID NO:6.

E24. The method of any one of E20-E23, wherein said BTN3A activating antibody comprises HCDRs1-3 of SEQ ID NO: 12-14 and LCDRs1-3 of SEQ ID NO: 15-17.

E25. The method of any one of E20-E24, wherein said BTN3A activating antibody comprises (a) a variable heavy chain (VH) polypeptide comprising an amino acid sequence that is at least about 95%, 96%, 97%, 98%, 99% or 100% identical to the amino acid sequence of SEQ ID NO: 1, and (b) a variable light chain (VL) polypeptide comprising an amino acid sequence that is at least about 95%, 96%, 97%, 98%, 99% or 100% identical to of SEQ ID NO: 2.

E26. The method of any one of E20-E25, wherein said BTN3A activating antibody is an antibody comprising or essentially consisting of a heavy chain of SEQ ID NO: 4 and a light chain of SEQ ID NO:6.

E27. The method of any one of E20-E26, wherein said BTN3A activating antibody comprises a mutant or chemically modified IgG1 constant region, wherein said mutant or chemically modified IgG1 constant region confers no or decreased binding to Fcγ receptors when compared to a corresponding antibody with wild type IgG1 isotype constant region.

E28. The method of any one of E20-E27, wherein said mutant IgG1 constant region is the IgG1 triple mutant L247F L248E and P350S.

E29. The method of any one of E20-E28, wherein said immune checkpoint inhibitor is a LAG-3 inhibitor, for example a monospecific antibody or bispecific antibody, or a fixed dose combination comprising an anti-LAG-3 antibody which: either,
comprises (a) a variable heavy chain (VH) polypeptide comprising an amino acid sequence that is at least about 95%, 96%, 97%, 98%, 99% or 100% identical to the SEQ ID NO: 37, and (b) a variable light chain (VL) polypeptide comprising an amino acid sequence that is at least about 95%, 96%, 97%, 98%, 99% or 100% identical to SEQ ID NO: 38;
comprises HCDRs1-3 of SEQ ID NO:45-47 and LCDRs1-3 of SEQ ID NO:48, 43 and 49 or,
competes for binding with an anti-LAG-3 antibody selected from relatlimab and favezelimab.

E30. The method according to any one of E20-E28, wherein said immune checkpoint inhibitor is a recombinant LAG-3 protein, for example a LAG-3-Ig fusion protein.

E31. The method of any one of E20-E30, wherein the anti-BTN3A antibody is administered at a unit dose ranging from about 1 to about 200 mg.

E32. The method of any one of E20-E31, wherein said immune checkpoint inhibitor is a LAG-3 inhibitor, for example, an anti-LAG-3 antibody administered at a dosage ranging from 100 to 1000 mg.

E33. The method of any one of E20-E31, wherein said immune checkpoint inhibitor is an anti-LAG-3/anti-PD-1 bispecific antibody, for example, tebotelimab.

E34. The method of any one of E20-E28, wherein said immune checkpoint inhibitor is a TIM-3 inhibitor, for example, sabatolimab.

E35. The method of any one of E20-E28, immune checkpoint inhibitor is an anti-TIM-3/anti-PD-1 bispecific antibody, for example, RO7121661.

E36. The method of any one of E20-E28, wherein said immune checkpoint inhibitor is a TIGIT inhibitor, for example, tiragolumab.

E37. The method of any one of E20-E28, wherein said immune checkpoint inhibitor is an anti-TIGIT/anti-PD-1 bispecific antibody, for example, AZD2936.

E38. The method of any one of E20-E37, wherein said cancer is selected from the group consisting of melanoma (e.g. metastatic malignant melanoma), lung cancer (e.g. non-small cell lung cancer), breast cancer, prostate cancer, head and neck cancer (e.g. squamous cell carcinoma of head and neck), renal cancer, colorectal cancer, gastric cancer, esophageal cancer, liver cancer, pancreatic cancer, glioblastoma, ovarian cancer, cervical cancer, cholangiocarcinoma, bladder cancer, cancer of the kidney or ureter, PD-L1 positive cancers and hematological malignancies including for example, acute myeloid leukemia, Hodgkin's lymphoma, multiple myeloma, diffuse large B-cell lymphoma (DLBCL), and indolent non-Hodgkin lymphoma (NHL).

E39. The method of any one of E20-E38, wherein said subject is refractory or relapse to PD-1 inhibitor therapy, for example subject refractory to Ipilimumab and/or nivolumab therapy.

E40. The method of any one of E20-E39, wherein said BTN3A activating antibody is administered once every three weeks, or once every four weeks.

E41. The method of any one of E20-E40, wherein the BTN3A activating antibody is administered intravenously, for example, at a unit dose of about 1 to about 200 mg, e.g., 75 mg, for example once every 21 days for 1 to 22 cycles.

E42. The method of any one of E20-E28, wherein said immune checkpoint inhibitor is an anti-LAG-3/anti-PD-1 bispecific antibody which is administered intravenously at a unit dose of about 100 to about 1000 mg, e.g., 600 mg, for example once every 21 days for 1 to 35 cycles.

E43. The method of any one of E20-E28, wherein said BTN3A antibody comprises a heavy chain of SEQ ID NO:4 and a light chain of SEQ ID NO:6, and said immune checkpoint inhibitor is eftilagimod alpha.

E44. The method of any one of E20-E28, wherein said BTN3A antibody comprises a heavy chain of SEQ ID NO:4 and a light chain of SEQ ID NO:6, and said immune checkpoint inhibitor is favezelimab.

E45. The method of any one of E20-E28, wherein said BTN3A antibody comprises a heavy chain of SEQ ID NO:4 and a light chain of SEQ ID NO:6, and said immune checkpoint inhibitor is the fixed dose combination nivolumab/relatlimab.

E46. The method of any one of E20-E28, wherein said BTN3A antibody comprises a heavy chain of SEQ ID NO:4 and a light chain of SEQ ID NO:6, and said immune checkpoint inhibitor is tebotelimab.

E47. The method of any one of E20-E28, wherein said BTN3A antibody comprises a heavy chain of SEQ ID NO:4 and a light chain of SEQ ID NO:6, and said immune checkpoint inhibitor is sabatolimab.

E48. The method of any one of E20-E28, wherein said BTN3A antibody comprises a heavy chain of SEQ ID NO:4 and a light chain of SEQ ID NO:6, and said immune checkpoint inhibitor is RO7121661.

E49. The method of any one of E20-E28, wherein said BTN3A antibody comprises a heavy chain of SEQ ID NO:4 and a light chain of SEQ ID NO:6, and said immune checkpoint inhibitor is tiragolumab.

E50. The method of any one of E20-E28, wherein said BTN3A antibody comprises a heavy chain of SEQ ID NO:4 and a light chain of SEQ ID NO:6, and said immune checkpoint inhibitor is AZD2936.

The disclosure having been fully described is now further illustrated by the following examples, which are illustrative only and are not meant to be further limiting.

TABLE 7
sequences used in the present disclosure

| SEQ ID | | Description of the | |
|---|---|---|---|
| NO: | # | sequence | sequence |
| 1 | aa | Humanized heavy | QVQLVQSGAEVKKPGASVKLSCKASGYIFTRYYMYWV |
| | | chain variable | KQRPGQGLEWIGEINPNNGGTKFNEKFKNRATLTVDK |
| | | region VH of ICT01 | SISTAYMELSRLRSDDTAVYYCSREDDYDGTPFAMDY |
| | | (mAb1) | WGQGTLVTVSS |
| 2 | aa | Humanized light | DIQMTQSPSSLSASVGDRVTITCHASQNINVWLSWYQ |
| | | chain variable | QKPGKAPKLLIYKASNLHTGVPSRFTGSGSGTDFTFTIS |
| | | region Vk1 of | SLQPEDIATYYCQQGQTYPYTFGQGTKLEIK |
| | | ICT01 (mAb1) | |
| 3 | aa | Humanized light | DIQMTQSPSSLSASVGDRVTITCHASQNINVWLSWYQ |
| | | chain variable | QKPGKAPKLLIYKASNLHTGVPSRFSGSGSGTDFTFTIS |
| | | region Vk2 of mAb2 | SLQPEDIATYYCQQGQTYPYTFGQGTKLEIK |
| 4 | aa | Full length heavy | QVQLVQSGAEVKKPGASVKLSCKASGYIFTRYYMYWV |
| | | chain of ICT01 and | KQRPGQGLEWIGEINPNNGGTKFNEKFKNRATLTVDK |
| | | mAb2 (with silent | SISTAYMELSRLRSDDTAVYYCSREDDYDGTPFAMDY |
| | | IgG1) | WGQGTLVTVSSASTKGPSVFPLAPSSKSTSGGTAALG |
| | | | CLVKDYFPEPVTVSWNSGALTSGVHTFPAVLQSSGLY |
| | | | SLSSVVTVPSSSLGTQTYICNVNHKPSNTKVDKRVEPK |
| | | | SCDKTHTCPPCPAPEFEGGPSVFLFPPKPKDTLMISRT |
| | | | PEVTCVVVDVSHEDPEVKFNWYVDGVEVHNAKTKPRE |
| | | | EQYNSTYRVVSVLTVLHQDWLNGKEYKCKVSNKALPA |
| | | | SIEKTISKAKGQPREPQVYTLPPSREEMTKNQVSLTCL |
| | | | VKGFYPSDIAVEWESNGQPENNYKTTPPVLDSDGSFF |
| | | | LYSKLTVDKSRWQQGNVFSCSVMHEALHNHYTQKSLS |
| | | | LSPG |
| 5 | aa | Full length heavy | QVQLVQSGAEVKKPGASVKLSCKASGYIFTRYYMYWV |
| | | chain of mAbs 4 | KQRPGQGLEWIGEINPNNGGTKFNEKFKNRATLTVDK |
| | | and 5 (with silent | SISTAYMELSRLRSDDTAVYYCSREDDYDGTPFAMDY |
| | | IgG4) | WGQGTLVTVSSASTKGPSVFPLAPCSRSTSESTAALG |
| | | | CLVKDYFPEPVTVSWNSGALTSGVHTFPAVLQSSGLY |
| | | | SLSSVVTVPSSSLGTKTYTCNVDHKPSNTKVDKRVESK |
| | | | YGPPCPPCPAPEFEGGPSVFLFPPKPKDTLMISRTPEV |
| | | | TCVVVDVSQEDPEVQFNWYVDGVEVHNAKTKPREEQ |
| | | | FNSTYRVVSVLTVLHQDWLNGKEYKCKVSNKGLPSSIE |
| | | | KTISKAKGQPREPQVYTLPPSQEEMTKNQVSLTCLVKG |
| | | | FYPSDIAVEWESNGQPENNYKTTPPVLDSDGSFFLYS |
| | | | RLTVDKSRWQEGNVFSCSVMHEALHNHYTQKSLSLSL |
| | | | G |
| 6 | aa | Full length light | DIQMTQSPSSLSASVGDRVTITCHASQNINVWLSWYQ |
| | | chain of ICT01 and | QKPGKAPKLLIYKASNLHTGVPSRFTGSGSGTDFTFTIS |
| | | mAb4 | SLQPEDIATYYCQQGQTYPYTFGQGTKLEIKRTVAAPS |
| | | | VFIFPPSDEQLKSGTASVVCLLNNFYPREAKVQWKVDN |
| | | | ALQSGNSQESVTEQDSKDSTYSLSSTLTLSKADYEKHK |
| | | | VYACEVTHQGLSSPVTKSFNRGEC |
| 7 | aa | Full length light | DIQMTQSPSSLSASVGDRVTITCHASQNINVWLSWYQ |
| | | chain of mAbs 2 | QKPGKAPKLLIYKASNLHTGVPSRFSGSGSGTDFTFTIS |
| | | and 5 | SLQPEDIATYYCQQGQTYPYTFGQGTKLEIKRTVAAPS |
| | | | VFIFPPSDEQLKSGTASVVCLLNNFYPREAKVQWKVDN |
| | | | ALQSGNSQESVTEQDSKDSTYSLSSTLTLSKADYEKHK |
| | | | VYACEVTHQGLSSPVTKSFNRGEC |
| 8 | nt | Full length heavy | CAGGTCCAACTGGTGCAGTCTGGGGCTGAAGTGAA |
| | | chain of ICT01 and | GAAGCCTGGGGCTTCAGTGAAGTTGTCCTGCAAGG |
| | | mAb 2 (silent IgG1) | CTTCTGGCTACATCTTCACCAGATACTATATGTATTG |
| | | | GGTGAAGCAGAGGCCTGGACAAGGCCTTGAGTGGA |
| | | | TTGGAGAGATTAATCCTAACAATGGTGGTACTAAGTT |
| | | | CAATGAGAAGTTCAAGAACAGGGCCACACTGACTGT |
| | | | AGACAAATCCATCAGCACAGCATACATGGAGCTCAG |
| | | | CAGGCTGAGATCTGACGACACGGCGGTCTATTATTG |
| | | | TTCAAGAGAGGATGATTACGACGGGACCCCCTTTGC |
| | | | TATGGACTACTGGGGTCAAGGAACCCTGGTCACCGT |
| | | | CTCCTCAGCCTCCACCAAGGGCCCATCGGTCTTCCC |
| | | | CCTGGCACCCTCCTCCAAGAGCACCTCTGGGGGCA |
| | | | CAGCGGCCCTGGGCTGCCTGGTCAAGGACTACTTC |
| | | | CCCGAACCGGTGACGGTGTCGTGGAACTCAGGCGC |
| | | | CCTGACCAGCGGCGTGCACACCTTCCCGGCTGTCC |
| | | | TACAGTCCTCAGGACTCTACTCCCTCAGCAGCGTGG |
| | | | TGACCGTGCCCTCCAGCAGCTTGGGCACCCAGACC |
| | | | TACATCTGCAACGTGAATCACAAGCCCAGCAACACC |
| | | | AAGGTGGACAAGAGAGTTGAGCCCAAATCTTGTGAC |
| | | | AAAACTCACACATGCCCACCGTGCCCAGCACCTGAA |
| | | | TTCGAGGGGGGACCGTCAGTCTTCCTCTTCCCCCCA |
| | | | AAACCCAAGGACACCCTCATGATCTCCCGGACCCCT |
| | | | GAGGTCACATGCGTGGTGGTGGACGTGAGCCACGA |
| | | | AGACCCTGAGGTCAAGTTCAACTGGTACGTGGACGG |
| | | | CGTGGAGGTGCATAATGCCAAGACAAAGCCGCGGG |
| | | | AGGAGCAGTACAACAGCACGTACCGTGTGGTCAGC |
| | | | GTCCTCACCGTCCTGCACCAGGACTGGCTGAATGG |
| | | | CAAGGAGTACAAGTGCAAGGTCTCCAACAAAGCCCT |
| | | | CCCAGCCTCCATCGAGAAAACCATCTCCAAAGCCAA |
| | | | AGGGCAGCCCCGAGAACCACAGGTGTACACCCTGC |
| | | | CCCCATCCCGGGAAGAGATGACCAAGAACCAGGTC |
| | | | AGCCTGACCTGCCTGGTCAAAGGCTTCTATCCCAGC |
| | | | GACATCGCCGTGGAGTGGGAGAGCAATGGGCAGCC |
| | | | GGAGAACAACTACAAGACCACGCCTCCCGTGCTGG |
| | | | ACTCCGACGGCTCCTTCTTCCTCTATAGCAAGCTCA |
| | | | CCGTGGACAAGAGCAGGTGGCAGCAGGGGAACGTC |
| | | | TTCTCATGCTCCGTGATGCATGAGGCTCTGCACAAC |
| | | | CACTACACGCAGAAGAGCCTCTCCCTGTCTCCGGGT |
| | | | TGA |
| 9 | nt | Full length heavy | CAGGTCCAACTGGTGCAGTCTGGGGCTGAAGTGAA |
| | | chain of mAbs 4 | GAAGCCTGGGGCTTCAGTGAAGTTGTCCTGCAAGG |
| | | and 5 (silent lgG4) | CTTCTGGCTACATCTTCACCAGATACTATATGTATTG |
| | | | GGTGAAGCAGAGGCCTGGACAAGGCCTTGAGTGGA |
| | | | TTGGAGAGATTAATCCTAACAATGGTGGTACTAAGTT |
| | | | CAATGAGAAGTTCAAGAACAGGGCCACACTGACTGT |
| | | | AGACAAATCCATCAGCACAGCATACATGGAGCTCAG |
| | | | CAGGCTGAGATCTGACGACACGGCGGTCTATTATTG |
| | | | TTCAAGAGAGGATGATTACGACGGGACCCCCTTTGC |
| | | | TATGGACTACTGGGGTCAAGGAACCCTGGTCACCGT |
| | | | CTCCTCAGCTTCCACCAAGGGCCCATCCGTCTTCCC |
| | | | CCTGGCGCCCTGCTCCAGGAGCACCTCCGAGAGCA |
| | | | CAGCCGCCCTGGGCTGCCTGGTCAAGGACTACTTC |
| | | | CCCGAACCGGTGACGGTGTCGTGGAACTCAGGCGC |
| | | | CCTGACCAGCGGCGTGCACACCTTCCCGGCTGTCC |
| | | | TACAGTCCTCAGGACTCTACTCCCTCAGCAGCGTGG |
| | | | TGACCGTGCCCTCCAGCAGCTTGGGCACGAAGACC |
| | | | TACACCTGCAATGTAGATCACAAGCCCAGCAACACC |
| | | | AAGGTGGACAAGAGAGTTGAGTCCAAATATGGTCCC |
| | | | CCATGCCCACCATGCCCAGCACCTGAGTTCGAGGG |
| | | | GGGACCATCAGTCTTCCTGTTCCCCCCAAAACCCAA |
| | | | GGACACTCTCATGATCTCCCGGACCCCTGAGGTCAC |
| | | | GTGCGTGGTGGTGGACGTGAGCCAGGAAGACCCCG |
| | | | AGGTCCAGTTCAACTGGTACGTGGATGGCGTGGAG |
| | | | GTGCATAATGCCAAGACAAAGCCGCGGGAGGAGCA |
| | | | GTTCAACAGCACGTACCGTGTGGTCAGCGTCCTCAC |
| | | | CGTCCTGCACCAGGACTGGCTGAACGGCAAGGAGT |
| | | | ACAAGTGCAAGGTCTCCAACAAAGGCCTCCCGTCCT |
| | | | CCATCGAGAAAACCATCTCCAAAGCCAAAGGGCAGC |
| | | | CCCGAGAGCCACAGGTGTACACCCTGCCCCCATCC |
| | | | CAGGAGGAGATGACCAAGAACCAGGTCAGCCTGAC |
| | | | CTGCCTGGTCAAAGGCTTCTACCCCAGCGACATCGC |
| | | | CGTGGAGTGGGAGAGCAATGGGCAGCCGGAGAACA |
| | | | ACTACAAGACCACGCCTCCCGTGCTGGACTCCGAC |
| | | | GGCTCCTTCTTCCTCTACAGCAGGCTAACCGTGGAC |
| | | | AAGAGCAGGTGGCAGGAGGGGAATGTCTTCTCATG |
| | | | CTCCGTGATGCATGAGGCTCTGCACAACCACTACAC |
| | | | ACAGAAGAGCCTCTCCCTGTCTCTGGGTTGA |
| 10 | nt | Full length light | GACATCCAGATGACCCAGTCTCCATCCAGTCTGTCT |
| | | chain of ICT01 and | GCATCCGTAGGAGACAGAGTCACCATCACTTGCCAT |
| | | mAb4 | GCCAGTCAGAACATTAATGTTTGGTTATCTTGGTACC |
| | | | AGCAGAAACCAGGAAAAGCCCCTAAACTCTTGATCT |
| | | | ATAAGGCTTCCAACTTGCACACAGGCGTCCCATCAA |
| | | | GATTTACTGGCAGTGGATCTGGAACAGATTTCACATT |
| | | | CACCATCAGCAGCCTGCAGCCTGAAGACATTGCCAC |
| | | | TTACTACTGTCAACAGGGTCAAACTTATCCATACACG |
| | | | TTCGGACAGGGGACCAAGCTGGAGATCAAACGAACT |
| | | | GTGGCTGCACCATCTGTCTTCATCTTCCCGCCATCT |
| | | | GATGAGCAGTTGAAATCTGGAACTGCCTCTGTTGTG |
| | | | TGCCTGCTGAATAACTTCTATCCCAGAGAGGCCAAA |
| | | | GTACAGTGGAAGGTGGATAACGCCCTCCAATCGGGT |
| | | | AACTCCCAGGAGAGTGTCACAGAGCAGGACAGCAA |
| | | | GGACAGCACCTACAGCCTCAGCAGCACCCTGACGC |
| | | | TGAGCAAAGCAGACTACGAGAAACACAAAGTCTACG |
| | | | CCTGCGAAGTCACCCATCAGGGCCTGAGCTCGCCC |
| | | | GTCACAAAGAGCTTCAACAGGGGAGAGTGTTAG |
| 11 | nt | Full length light | GACATCCAGATGACCCAGTCTCCATCCAGTCTGTCT |
| | | chain of mAb 2 and | GCATCCGTAGGAGACAGAGTCACCATCACTTGCCAT |
| | | 5 | GCCAGTCAGAACATTAATGTTTGGTTATCTTGGTACC |
| | | | AGCAGAAACCAGGAAAAGCCCCTAAACTCTTGATCT |
| | | | ATAAGGCTTCCAACTTGCACACAGGCGTCCCATCAA |
| | | | GATTTAGTGGCAGTGGATCTGGAACAGATTTCACATT |
| | | | CACCATCAGCAGCCTGCAGCCTGAAGACATTGCCAC |
| | | | TTACTACTGTCAACAGGGTCAAACTTATCCATACACG |
| | | | TTCGGACAGGGGACCAAGCTGGAGATCAAACGAACT |
| | | | GTGGCTGCACCATCTGTCTTCATCTTCCCGCCATCT |
| | | | GATGAGCAGTTGAAATCTGGAACTGCCTCTGTTGTG |
| | | | TGCCTGCTGAATAACTTCTATCCCAGAGAGGCCAAA |
| | | | GTACAGTGGAAGGTGGATAACGCCCTCCAATCGGGT |
| | | | AACTCCCAGGAGAGTGTCACAGAGCAGGACAGCAA |
| | | | GGACAGCACCTACAGCCTCAGCAGCACCCTGACGC |
| | | | TGAGCAAAGCAGACTACGAGAAACACAAAGTCTACG |
| | | | CCTGCGAAGTCACCCATCAGGGCCTGAGCTCGCCC |
| | | | GTCACAAAGAGCTTCAACAGGGGAGAGTGTTAG |
| 12 | aa | HCDR 1 of mAb 7.2, | RYYMY |
| | | ICT01, mAb 2, 4 | |
| | | and 5 | |
| 13 | aa | HCDR2 of mAb 7.2, | EINPNNGGTKFNEKFKN |
| | | ICT01, mAb2, | |
| | | mAb4 and mAb5 | |
| 14 | aa | HCDR3 of mAb 7.2, | EDDYDGTPFAMDY |
| | | ICT01, mAb2, | |
| | | mAb4 and mAb5 | |
| 15 | aa | LCDR1 of mAb 7.2, | HASQNINVWLS |
| | | ICT01, mAb2, | |
| | | mAb4 and mAb5 | |
| 16 | aa | LCDR2 of mAb 7.2, | KASNLHT |
| | | ICT01, mAb2, | |
| | | mAb4 and mAb5 | |
| 17 | aa | LCDR3 of mAb 7.2, | QQGQTYPYT |
| | | ICT01, mAb2, | |
| | | mAb4 and mAb5 | |
| 18 | aa | HCDR1 of mAb | RYYLY |
| | | 20.1 | |
| 19 | aa | HCDR2 of mAb | EINPNNGGTKFNEKFKS |
| | | 20.1 | |
| 20 | aa | HCDR3 of mAb | EDDYDGTPDAMDY |
| | | 20.1 | |
| 21 | aa | LCDR1 of mAb | HASQNINLWLS |
| | | 20.1 | |
| 22 | aa | LCDR2 of mAb | RASNLHT |
| | | 20.1 | |
| 23 | aa | LCDR3 of mAb | QQGHSYPYT |
| | | 20.1 | |
| 24 | aa | Human BTN3A1 | MKMASFLAFLLLNFRVCLLLLQLLMPHSAQFSVLGPSG |
| | | | PILAMVGEDADLPCHLFPTMSAETMELKWVSSSLRQV |
| | | | VNVYADGKEVEDRQSAPYRGRTSILRDGITAGKAALRI |
| | | | HNVTASDSGKYLCYFQDGDFYEKALVELKVAALGSDL |
| | | | HVDVKGYKDGGIHLECRSTGWYPQPQIQWSNNKGENI |
| | | | PTVEAPVVADGVGLYAVAASVIMRGSSGEGVSCTIRSS |
| | | | LLGLEKTASISIADPFFRSAQRWIAALAGTLPVLLLLLGG |
| | | | AGYFLWQQQEEKKTQFRKKKREQELREMAWSTMKQE |
| | | | QSTRVKLLEELRWRSIQYASRGERHSAYNEWKKALFK |
| | | | PADVILDPKTANPILLVSEDQRSVQRAKEPQDLPDNPE |
| | | | RFNWHYCVLGCESFISGRHYWEVEVGDRKEWHIGVC |
| | | | SKNVQRKGWVKMTPENGFWTMGLTDGNKYRTLTEPR |
| | | | TNLKLPKPPKKVGVFLDYETGDISFYNAVDGSHIHTFLD |
| | | | VSFSEALYPVFRILTLEPTALTICPA |
| 25 | aa | Human BTN3A2 | MKMASSLAFLLLNFHVSLLLVQLLTPCSAQFSVLGPSG |
| | | | PILAMVGEDADLPCHLFPTMSAETMELKWVSSSLRQV |
| | | | VNVYADGKEVEDRQSAPYRGRTSILRDGITAGKAALRI |
| | | | HNVTASDSGKYLCYFQDGDFYEKALVELKVAALGSNL |
| | | | HVEVKGYEDGGIHLECRSTGWYPQPQIQWSNAKGENI |
| | | | PAVEAPVVADGVGLYEVAASVIMRGGSGEGVSCIIRNS |
| | | | LLGLEKTASISIADPFFRSAQPWIAALAGTLPILLLLLAGA |
| | | | SYFLWRQQKEITALSSEIESEQEMKEMGYAATEREISL |
| | | | RESLQEELKRKKIQYLTRGEESSSDTNKSA |
| 26 | aa | Human BTN3A3 | MKMASSLAFLLLNFHVSLFLVQLLTPCSAQFSVLGPSG |
| | | | PILAMVGEDADLPCHLFPTMSAETMELRWVSSSLRQV |
| | | | VNVYADGKEVEDRQSAPYRGRTSILRDGITAGKAALRI |
| | | | HNVTASDSGKYLCYFQDGDFYEKALVELKVAALGSDL |
| | | | HIEVKGYEDGGIHLECRSTGWYPQPQIKWSDTKGENIP |
| | | | AVEAPVVADGVGLYAVAASVIMRGSSGGGVSCIIRNSL |
| | | | LGLEKTASISIADPFFRSAQPWIAALAGTLPISLLLLAGA |
| | | | SYFLWRQQKEKIALSRETEREREMKEMGYAATEQEISL |
| | | | REKLQEELKWRKIQYMARGEKSLAYHEWKMALFKPAD |
| | | | VILDPDTANAILLVSEDQRSVQRAEEPRDLPDNPERFE |
| | | | WRYCVLGCENFTSGRHYWEVEVGDRKEWHIGVCSKN |
| | | | VERKKGWVKMTPENGYWTMGLTDGNKYRALTEPRTN |
| | | | LKLPEPPRKVGIFLDYETGEISFYNATDGSHIYTFPHAS |
| | | | FSEPLYPVFRILTLEPTALTICPIPKEVESSPDPDLVPDH |
| | | | SLETPLTPGLANESGEPQAEVTSLLLPAHPGAEVSPSA |
| | | | TTNQNHKLQARTEALY |
| 27 | aa | Cynomolgus | MGSSLAFLLLSFHVCVLLLQLLMPHSAQFAVVGPPGPIL |
| | | macaque (m. | AMVGEDADLPCHLFPTMSAETMELRWVSSNLRQVVN |
| | | fascicularis) | VYADGKEVEDRQSAAYRGRTSILRDGITAGKAALRIHN |
| | | BTN3A1 | VTASDSGKYLCYFQDGDFYEKALVELKVAALGSDLHID |
| | | ectodomain used | VKGYEDGGIHLECRSTGWYPQPQIRWSNDKGENIPAV |
| | | for recombinant | EAPVFVDGVGLYAVAASVILRGSSGEGVSCTIRSSLLG |
| | | protein production | LEKTTSISIAG |
| 28 | aa | Cynomolgus | MGSSLAFLLLNFHVSFFLVQLLTPCSAQFSVLGPSGPIL |
| | | macaque (m. | AMVGEDADLPCHLFPTMSAETMELRWVSSSLRQVVN |
| | | fascicularis) | VYADGKEVEDRQSAPYRGRTSILRDDIAAGKAALRIHN |
| | | BTN3A2 | VTASDSGKYLCYFQDADFYEKALVELKVAALGSNLHVE |
| | | ectodomain used | VKGYEDGGIHLECRSTGWYPQPKIQWSNAKGQNIPAV |
| | | for recombinant | EAPVVADGVGLYAVAASVIMRGGSGESVSCIIRNSVLG |
| | | protein production | LEKTASISIAD |
| 29 | aa | Cynomolgus | MANFLAFLLLNFRVCLLLVQLLTPCSAQFAVLGPHGPIL |
| | | macaque (m. | AMVGEDVDLPCHLFPTMSAETMELRWVSSSLRQVVN |
| | | fascicularis) | VYSDGKEVEDRQSAPYRGRTSILRDGITAGKAALRIHN |
| | | BTN3A3 | VTASDSGKYLCYFQDGDFYEKALVELKVAALGSDLHIE |
| | | ectodomain used | VKGYEDGGIHLECRSTGWYPQPQIQWSNTKGQHIPAV |
| | | for recombinant | KAPVVADGVGLYAVAASVIMRGSSGEGVSCIIRNSLLG |
| | | protein production | LEKTASISITD |
| 30 | aa | Eftilagimod alpha | LQPGAEVPVVWAQEGAPAQLPCSPTIPLQDLSLLRRA |
| | | (IMP321) (Lag-3- | GVTWQHQPDSGPPAAAPGHPLAPGPHPAAPSSWGP |
| | | Fc fusion | RPRRYTVLSVGPGGLRSGRLPLQPRVQLDERGRQRG |
| | | sequence) | DFSLWLRPARRADAGEYRAAVHLRDRALSCRLRLRLG |
| | | | QASMTASPPGSLRASDWVILNCSFSRPDRPASVHWFR |
| | | | NRGQGRVPVRESPHHHLAESFLFLPQVSPMDSGPWG |
| | | | CILTYRDGFNVSIMYNLTVLGLEPPTPLTVYAGAGSRV |
| | | | GLPCRLPAGVGTRSFLTAKWTPPGGGPDLLVTGDNGD |
| | | | FTLRLEDVSQAQAGTYTCHIHLQEQQLNATVTLAIITVT |
| | | | PKSFGSPGSLGKLLCEVTPVSGQERFVWSSLDTPSQR |
| | | | SFSGPWLEAQEAQLLSQPWQCQLYQGERLLGAAVYF |
| | | | TELSSPGDDDDKGSGSGEPKSCDKTHTCPPCPAPELL |
| | | | GGPSVFLFPPKPKDTLMISRTPEVTCVVVDVSHEDPEV |
| | | | KFNWYVDGVEVHNAKTKPREEQYNSTYRVVSVLTVLH |
| | | | QDWLNGKEYKCKVSNKALPAPIEKTISKAKGQPREPQV |
| | | | YTLPPSRDELTKNQVSLTCLVKGFYPSDIAVEWESNGQ |
| | | | PENNYKTTPPVLDSDGSFFLYSKLTVDKSRWQQGNVF |
| | | | SCSVMHEALHNHYTQKSLSLSPGK |
| 31 | aa | Opdualag | QVQLVESGGGVVQPGRSLRLDCKASGITFSNSGMHW |
| | | Sequence 1 Full | VRQAPGKGLEWVAVIWYDGSKRYYADSVKGRFTISRD |
| | | length heavy chain | NSKNTLFLQMNSLRAEDTAVYYCATNDDYWGQGTLVT |
| | | of Nivolumab | VSSASTKGPSVFPLAPCSRSTSESTAALGCLVKDYFPE |
| | | | PVTVSWNSGALTSGVHTFPAVLQSSGLYSLSSVVTVP |
| | | | SSSLGTKTYTCNVDHKPSNTKVDKRVESKYGPPCPPC |
| | | | PAPEFLGGPSVFLFPPKPKDTLMISRTPEVTCVVVDVS |
| | | | QEDPEVQFNWYVDGVEVHNAKTKPREEQFNSTYRVV |
| | | | SVLTVLHQDWLNGKEYKCKVSNKGLPSSIEKTISKAKG |
| | | | QPREPQVYTLPPSQEEMTKNQVSLTCLVKGFYPSDIAV |
| | | | EWESNGQPENNYKTTPPVLDSDGSFFLYSRLTVDKSR |
| | | | WQEGNVFSCSVMHEALHNHYTQKSLSLSLGK |
| 32 | aa | Opdualag | EIVLTQSPATLSLSPGERATLSCRASQSVSSYLAWYQQ |
| | | Sequence 2 Full | KPGQAPRLLIYDASNRATGIPARFSGSGSGTDFTLTISS |
| | | length light chain of | LEPEDFAVYYCQQSSNWPRTFGQGTKVEIKRTVAAPS |
| | | Nivolumab | VFIFPPSDEQLKSGTASVVCLLNNFYPREAKVQWKVDN |
| | | | ALQSGNSQESVTEQDSKDSTYSLSSTLTLSKADYEKHK |
| | | | VYACEVTHQGLSSPVTKSFNRGEC |
| 33 | aa | Opdualag | QVQLQQWGAGLLKPSETLSLTCAVYGGSFSDYYWNW |
| | | Sequence 3 Full | IRQPPGKGLEWIGEINHRGSTNSNPSLKSRVTLSLDTS |
| | | length heavy chain | KNQFSLKLRSVTAADTAVYYCAFGYSDYEYNWFDPW |
| | | of Relatlimab | GQGTLVTVSSASTKGPSVFPLAPCSRSTSESTAALGCL |
| | | | VKDYFPEPVTVSWNSGALTSGVHTFPAVLQSSGLYSL |
| | | | SSVVTVPSSSLGTKTYTCNVDHKPSNTKVDKRVESKY |
| | | | GPPCPPCPAPEFLGGPSVFLFPPKPKDTLMISRTPEVT |
| | | | CVVVDVSQEDPEVQFNWYVDGVEVHNAKTKPREEQF |
| | | | NSTYRVVSVLTVLHQDWLNGKEYKCKVSNKGLPSSIEK |
| | | | TISKAKGQPREPQVYTLPPSQEEMTKNQVSLTCLVKGF |
| | | | YPSDIAVEWESNGQPENNYKTTPPVLDSDGSFFLYSRL |
| | | | TVDKSRWQEGNVFSCSVMHEALHNHYTQKSLSLSLGK |
| 34 | aa | Opdualag | EIVLTQSPATLSLSPGERATLSCRASQSISSYLAWYQQ |
| | | Sequence 4 Full | KPGQAPRLLIYDASNRATGIPARFSGSGSGTDFTLTISS |
| | | length light chain of | LEPEDFAVYYCQQRSNWPLTFGQGTNLEIKRTVAAPS |
| | | Relatlimab | VFIFPPSDEQLKSGTASVVCLLNNFYPREAKVQWKVDN |
| | | | ALQSGNSQESVTEQDSKDSTYSLSSTLTLSKADYEKHK |
| | | | VYACEVTHQGLSSPVTKSFNRGEC |
| 35 | aa | VH of Nivolumab | QVQLVESGGGVVQPGRSLRLDCKASGITFSNSGMHW |
| | | | VRQAPGKGLEWVAVIWYDGSKRYYADSVKGRFTISRD |
| | | | NSKNTLFLQMNSLRAEDTAVYYCATNDDYWGQGTLVT |
| | | | VSS |
| 36 | aa | VL of Nivolumab | EIVLTQSPATLSLSPGERATLSCRASQSVSSYLAWYQQ |
| | | | KPGQAPRLLIYDASNRATGIPARFSGSGSGTDFTLTISS |
| | | | LEPEDFAVYYCQQSSNWPRTFGQGTKVEIK |
| 37 | aa | VH of Relatlimab | QVQLQQWGAGLLKPSETLSLTCAVYGGSFSDYYWNW |
| | | | IRQPPGKGLEWIGEINHRGSTNSNPSLKSRVTLSLDTS |
| | | | KNQFSLKLRSVTAADTAVYYCAFGYSDYEYNWFDPW |
| | | | GQGTLVTVSS |
| 38 | aa | VL of Relatlimab | EIVLTQSPATLSLSPGERATLSCRASQSISSYLAWYQQ |
| | | | KPGQAPRLLIYDASNRATGIPARFSGSGSGTDFTLTISS |
| | | | LEPEDFAVYYCQQRSNWPLTFGQGTNLEIK |
| 39 | aa | HCDR1 of | NSGMH |
| | | Nivolumab | |
| 40 | aa | HCDR2 of | VIWYDGSKRYYADSVKG |
| | | Nivolumab | |
| 41 | aa | HCDR3 of | NDDY |
| | | Nivolumab | |
| 42 | aa | LCDR1 of | RASQSVSSYLA |
| | | Nivolumab | |
| 43 | aa | LCDR2 of | DASNRAT |
| | | Nivolumab | |
| 44 | aa | LCDR3 of | QQSSNWPRT |
| | | Nivolumab | |
| 45 | aa | HCDR 1 of | GGSFSDYY |
| | | Relatlimab | |
| 46 | aa | HCDR2 of | EINHRGSTNSNPSLKS |
| | | Relatlimab | |
| 47 | aa | HCDR3 of | GYSDYEYNWFDP |
| | | Relatlimab | |
| 48 | aa | LCDR1 of | RASQSISSYLA |
| | | Relatlimab | |
| — | aa | LCDR2 of | DASNRAT same as SEQ ID NO: 43 |
| | | Relatlimab | |
| 49 | aa | LCDR3 of | QQRSNWPLT |
| | | Relatlimab | |
| 50 | aa | MK-4280- | QMQLVQSGPEVKKPGTSVKVSCKASGYTFTDYNVDW |
| | | Favezelimab-full | VRQARGQRLEWIGDINPNDGGTIYAQKFQERVTITVDK |
| | | heavy chain | STSTAYMELSSLRSEDTAVYYCARNYRWFGAMDHWG |
| | | | QGTTVTVSSASTKGPSVFPLAPCSRSTSESTAALGCLV |
| | | | KDYFPEPVTVSWNSGALTSGVHTFPAVLQSSGLYSLS |
| | | | SVVTVPSSSLGTKTYTCNVDHKPSNTKVDKRVESKYG |
| | | | PPCPPCPAPEFLGGPSVFLFPPKPKDTLMISRTPEVTC |
| | | | VVVDVSQEDPEVQFNWYVDGVEVHNAKTKPREEQFN |
| | | | STYRVVSVLTVLHQDWLNGKEYKCKVSNKGLPSSIEKT |
| | | | ISKAKGQPREPQVYTLPPSQEEMTKNQVSLTCLVKGFY |
| | | | PSDIAVEWESNGQPENNYKTTPPVLDSDGSFFLYSRLT |
| | | | VDKSRWQEGNVFSCSVMHEALHNHYTQKSLSLSLGK |
| 51 | aa | MK-4280- | DIVMTQTPLSLSVTPGQPASISCKASQSLDYEGDSDMN |
| | | Favezelimab-full | WYLQKPGQPPQLLIYGASNLESGVPDRFSGSGSGTDF |
| | | light chain | TLKISRVEAEDVGVYYCQQSTEDPRTFGGGTKVEIKRT |
| | | | VAAPSVFIFPPSDEQLKSGTASVVCLLNNFYPREAKVQ |
| | | | WKVDNALQSGNSQESVTEQDSKDSTYSLSSTLTLSKA |
| | | | DYEKHKVYACEVTHQGLSSPVTKSFNRGEC |
| 52 | aa | MK-4280- | QMQLVQSGPEVKKPGTSVKVSCKASGYTFTDYNVDW |
| | | Favezelimab-VH | VRQARGQRLEWIGDINPNDGGTIYAQKFQERVTITVDK |
| | | | STSTAYMELSSLRSEDTAVYYCARNYRWFGAMDHWG |
| | | | QGTTVTVSS |
| 53 | aa | MK-4280- | DIVMTQTPLSLSVTPGQPASISCKASQSLDYEGDSDMN |
| | | Favezelimab-VL | WYLQKPGQPPQLLIYGASNLESGVPDRFSGSGSGTDF |
| | | | TLKISRVEAEDVGVYYCQQSTEDPRTFGGGTKVEIK |
| 54 | aa | HCDR1 of | DYNVD |
| | | Favezelimab | |
| 55 | aa | HCDR2 of | DINPNDGGTIYAQKFQE |
| | | Favezelimab | |
| 56 | aa | HCDR3 of | NYRWFGAMDH |
| | | Favezelimab | |
| 57 | aa | LCDR1 of | KASQSLDYEGDSDMN |
| | | Favezelimab | |
| 58 | aa | LCDR2 of | GASNLES |
| | | Favezelimab | |
| 59 | aa | LCDR3 of | QQSTEDPRT |
| | | Favezelimab | |
| 60 | aa | Tebotelimab- | EIVLTQSPATLSLSPGERATLSCRASESVDNYGMSFMN |
| | | Polypeptide 1 | WFQQKPGQPPKLLIHAASNQGSGVPSRFSGSGSGTD |
| | | | FTLTISSLEPEDFAVYFCQQSKEVPYTFGGGTKVEIKG |
| | | | GGSGGGGQVQLVQSGAEVKKPGASVKVSCKASGYTF |
| | | | TDYNMDWVRQAPGQGLEWMGDINPDNGVTIYNQKFE |
| | | | GRVTMTTDTSTSTAYMELRSLRSDDTAVYYCAREADY |
| | | | FYFDYWGQGTTLTVSSGGCGGGKVAACKEKVAALKE |
| | | | KVAALKEKVAALKE |
| 61 | aa | Tebotelimab- | DIQMTQSPSSLSASVGDRVTITCRASQDVSSVVAWYQ |
| | | Polypeptide 2 | QKPGKAPKLLIYSASYRYTGVPSRFSGSGSGTDFTLTIS |
| | | | SLQPEDFATYYCQQHYSTPWTFGGGTKLEIKGGGSGG |
| | | | GGQVQLVQSGAEVKKPGASVKVSCKASGYSFTSYWM |
| | | | NWVRQAPGQGLEWIGVIHPSDSETWLDQKFKDRVTIT |
| | | | VDKSTSTAYMELSSLRSEDTAVYYCAREHYGTSPFAY |
| | | | WGQGTLVTVSSGGCGGGEVAACEKEVAALEKEVAAL |
| | | | EKEVAALEKESKYGPPCPPCPAPEFLGGPSVFLFPPKP |
| | | | KDTLYITREPEVTCVVVDVSQEDPEVQFNWYVDGVEV |
| | | | HNAKTKPREEQFNSTYRVVSVLTVLHQDWLNGKEYKC |
| | | | KVSNKGLPSSIEKTISKAKGQPREPQVYTLPPSQEEMT |
| | | | KNQVSLTCLVKGFYPSDIAVEWESNGQPENNYKTTPP |
| | | | VLDSDGSFFLYSRLTVDKSRWQEGNVFSCSVMHEALH |
| | | | NHYTQKSLSLSLG |
| 62 | aa | Tebotelimab-VH | QVQLVQSGAEVKKPGASVKVSCKASGYTFTDYNMDW |
| | | Polypeptide 1 | VRQAPGQGLEWMGDINPDNGVTIYNQKFEGRVTMTT |
| | | | DTSTSTAYMELRSLRSDDTAVYYCAREADYFYFDYWG |
| | | | QGTTLTVSS |
| 63 | aa | Tebotelimab-VL | EIVLTQSPATLSLSPGERATLSCRASESVDNYGMSFMN |
| | | Polypeptide 2 | WFQQKPGQPPKLLIHAASNQGSGVPSRFSGSGSGTD |
| | | | FTLTISSLEPEDFAVYFCQQSKEVPYTFGGGTKVEIK |
| 64 | aa | Tebotelimab-VH | DIQMTQSPSSLSASVGDRVTITCRASQDVSSVVAWYQ |
| | | Polypeptide 3 | QKPGKAPKLLIYSASYRYTGVPSRFSGSGSGTDFTLTIS |
| | | | SLQPEDFATYYCQQHYSTPWTFGGGTKLEIK |
| 65 | aa | Tebotelimab- | DYNMD |
| | | HCDR1 of | |
| | | polypeptide 1 | |
| 66 | aa | Tebotelimab- | DINPDNGVTIYNQKFEG |
| | | HCDR2 of | |
| | | polypeptide 1 | |
| 67 | aa | Tebotelimab- | EADYFYFDY |
| | | HCDR3 of | |
| | | polypeptide 1 | |
| 68 | aa | Tebotelimab- | RASESVDNYGMSFMN |
| | | LCDR1 of | |
| | | polypeptide 2 | |
| 69 | aa | Tebotelimab- | AASNQGS |
| | | LCDR2 of | |
| | | polypeptide 2 | |
| 70 | aa | Tebotelimab- | QQSKEVPYT |
| | | LCDR3 of | |
| | | polypeptide 2 | |
| 71 | aa | Tebotelimab- | SYWMN |
| | | HCDR1 of | |
| | | polypeptide 3 | |
| 72 | aa | Tebotelimab- | VIHPSDSETWLDQKFKD |
| | | HCDR2of | |
| | | polypeptide 3 | |
| 73 | aa | Tebotelimab- | EHYGTSPFAY |
| | | HCDR3 of | |
| | | polypeptide 3 | |
| 74 | aa | Tebotelimab- | RASQDVSSVVA |
| | | LCDR1 of | |
| | | polypeptide 4 | |
| 75 | aa | Tebotelimab- | SASYRYT |
| | | LCDR2of | |
| | | polypeptide 4 | |
| 76 | aa | Tebotelimab- | HYSTPWT |
| | | LCDR3 of | |
| | | polypeptide 4 | |
| 77 | aa | Sabatolimab (anti- | QVQLVQSGAEVKKPGSSVKVSCKASGYTFTSYNMHW |
| | | TIM-3)-Full heavy | VRQAPGQGLEWMGDIYPGNGDTSYNQKFKGRVTITAD |
| | | chain | KSTSTVYMELSSLRSEDTAVYYCARVGGAFPMDYWG |
| | | | QGTTVTVSSASTKGPSVFPLAPCSRSTSESTAALGCLV |
| | | | KDYFPEPVTVSWNSGALTSGVHTFPAVLQSSGLYSLS |
| | | | SVVTVPSSSLGTKTYTCNVDHKPSNTKVDKRVESKYG |
| | | | PPCPPCPAPEFLGGPSVFLFPPKPKDTLMISRTPEVTC |
| | | | VVVDVSQEDPEVQFNWYVDGVEVHNAKTKPREEQFN |
| | | | STYRVVSVLTVLHQDWLNGKEYKCKVSNKGLPSSIEKT |
| | | | ISKAKGQPREPQVYTLPPSQEEMTKNQVSLTCLVKGFY |
| | | | PSDIAVEWESNGQPENNYKTTPPVLDSDGSFFLYSRLT |
| | | | VDKSRWQEGNVFSCSVMHEALHNHYTQKSLSLSLG |
| 78 | aa | Sabatolimab (anti- | AIQLTQSPSSLSASVGDRVTITCRASESVEYYGTSLMQ |
| | | TIM-3)-Full light | WYQQKPGKAPKLLIYAASNVESGVPSRFSGSGSGTDF |
| | | chain | TLTISSLQPEDFATYFCQQSRKDPSTFGGGTKVEIKRT |
| | | | VAAPSVFIFPPSDEQLKSGTASVVCLLNNFYPREAKVQ |
| | | | WKVDNALQSGNSQESVTEQDSKDSTYSLSSTLTLSKA |
| | | | DYEKHKVYACEVTHQGLSSPVTKSFNRGEC |
| 79 | aa | Sabatolimab (anti- | QVQLVQSGAEVKKPGSSVKVSCKASGYTFTSYNMHW |
| | | TIM-3)-VH | VRQAPGQGLEWMGDIYPGNGDTSYNQKFKGRVTITAD |
| | | | KSTSTVYMELSSLRSEDTAVYYCARVGGAFPMDYWG |
| | | | QGTTVTVSS |
| 80 | aa | Sabatolimab (anti- | AIQLTQSPSSLSASVGDRVTITCRASESVEYYGTSLMQ |
| | | TIM-3)-VL | WYQQKPGKAPKLLIYAASNVESGVPSRFSGSGSGTDF |
| | | | TLTISSLQPEDFATYFCQQSRKDPSTFGGGTKVEIK |
| 81 | aa | Sabatolimab- | SYNMH |
| | | HCDR1 | |
| 82 | aa | Sabatolimab- | DIYPGNGDTSYNQKFKG |
| | | HCDR2 | |
| 83 | aa | Sabatolimab- | VGGAFPMDY |
| | | HCDR3 | |
| 84 | aa | Sabatolimab- | RASESVEYYGTSLMQ |
| | | LCDR1 | |
| 85 | aa | Sabatolimab- | AASNVES |
| | | LCDR2 | |
| 86 | aa | Sabatolimab- | QQSRKDPST |
| | | LCDR3 | |
| 87 | aa | RO7121661 (TIM-3 | DIVMTQSPDSLAVSLGERATINCKASESVDTSDNSFIH |
| | | x PD-1 bispecific | WYQQKPGQSPKLLIYRSSTLESGVPDRFSGSGSGTDF |
| | | antibody) Full | TLTISSLQAEDVAVYYCQQNYDVPWTFGQGTKVEIKSS |
| | | Polypeptide 1 | ASTKGPSVFPLAPSSKSTSGGTAALGCLVKDYFPEPVT |
| | | | VSWNSGALTSGVHTFPAVLQSSGLYSLSSVVTVPSSSL |
| | | | GTQTYICNVNHKPSNTKVDKKVEPKSCDKTHTCPPCP |
| | | | APEAAGGPSVFLFPPKPKDTLMISRTPEVTCVVVDVSH |
| | | | EDPEVKFNWYVDGVEVHNAKTKPREEQYNSTYRVVS |
| | | | VLTVLHQDWLNGKEYKCKVSNKALGAPIEKTISKAKGQ |
| | | | PREPQVYTLPPCRDELTKNQVSLWCLVKGFYPSDIAVE |
| | | | WESNGQPENNYKTTPPVLDSDGSFFLYSKLTVDKSRW |
| | | | QQGNVFSCSVMHEALHNHYTQKSLSLSPGK |
| 88 | aa | RO7121661 (TIM-3 | EVQLLESGGGLVQPGGSLRLSCAASGFSFSSYTMSWV |
| | | x PD-1 bispecific | RQAPGKGLEWVATISGGGRDIYYPDSVKGRFTISRDNS |
| | | antibody) Full | KNTLYLQMNSLRAEDTAVYYCVLLTGRVYFALDSWGQ |
| | | Polypeptide 2 | GTLVTVSSASVAAPSVFIFPPSDEQLKSGTASVVCLLN |
| | | | NFYPREAKVQWKVDNALQSGNSQESVTEQDSKDSTY |
| | | | SLSSTLTLSKADYEKHKVYACEVTHQGLSSPVTKSFNR |
| | | | GEC |
| 89 | aa | RO7121661 (TIM-3 | EVQLVESGGGLVQPGGSLRLSCAASGFNIKTTYMHWV |
| | | x PD-1 bispecific | RQAPGKGLEWVGRIDPADDNTKYAPKFQGKATISADT |
| | | antibody) Full | SKNTAYLQMNSLRAEDTAVYYCVRDFGYVAWFAYWG |
| | | Polypeptide 3 | QGTLVTVSSASTKGPSVFPLAPSSKSTSGGTAALGCLV |
| | | | EDYFPEPVTVSWNSGALTSGVHTFPAVLQSSGLYSLS |
| | | | SVVTVPSSSLGTQTYICNVNHKPSNTKVDEKVEPKSCD |
| | | | KTHTCPPCPAPEAAGGPSVFLFPPKPKDTLMISRTPEV |
| | | | TCVVVDVSHEDPEVKFNWYVDGVEVHNAKTKPREEQ |
| | | | YNSTYRVVSVLTVLHQDWLNGKEYKCKVSNKALGAPIE |
| | | | KTISKAKGQPREPQVCTLPPSRDELTKNQVSLSCAVKG |
| | | | FYPSDIAVEWESNGQPENNYKTTPPVLDSDGSFFLVSK |
| | | | LTVDKSRWQQGNVFSCSVMHEALHNHYTQKSLSLSP |
| | | | GK |
| 90 | aa | RO7121661 (TIM-3 | DIVMTQSPLSLPVTPGEPASISCRASQSVDNYVAWYLQ |
| | | x PD-1 bispecific | KPGQSPQLLIYYASNRYIGVPDRFSGSGSGTDFTLKISR |
| | | antibody) Full | VEAEDVGVYYCQQHYSSPYTFGQGTKVEIKRTVAAPS |
| | | Polypeptide 4 | VFIFPPSDRKLKSGTASVVCLLNNFYPREAKVQWKVDN |
| | | | ALQSGNSQESVTEQDSKDSTYSLSSTLTLSKADYEKHK |
| | | | VYACEVTHQGLSSPVTKSFNRGEC |
| 91 | aa | RO7121661 (TIM-3 | DIVMTQSPDSLAVSLGERATINCKASESVDTSDNSFIH |
| | | x PD-1 bispecific | WYQQKPGQSPKLLIYRSSTLESGVPDRFSGSGSGTDF |
| | | antibody) VH | TLTISSLQAEDVAVYYCQQNYDVPWTFGQGTKVEIKSS |
| | | polypetide 1 | |
| 92 | aa | RO7121661 (TIM-3 | EVQLLESGGGLVQPGGSLRLSCAASGFSFSSYTMSWV |
| | | x PD-1 bispecific | RQAPGKGLEWVATISGGGRDIYYPDSVKGRFTISRDNS |
| | | antibody) VL | KNTLYLQMNSLRAEDTAVYYCVLLTGRVYFALDSWGQ |
| | | polypeptide 2 | GTLVTVSS |
| 93 | aa | RO7121661 (TIM-3 | EVQLVESGGGLVQPGGSLRLSCAASGFNIKTTYMHWV |
| | | x PD-1 bispecific | RQAPGKGLEWVGRIDPADDNTKYAPKFQGKATISADT |
| | | antibody) VH | SKNTAYLQMNSLRAEDTAVYYCVRDFGYVAWFAYWG |
| | | polypeptide 3 | QGTLVTVSS |
| 94 | aa | RO7121661 (TIM-3 | DIVMTQSPLSLPVTPGEPASISCRASQSVDNYVAWYLQ |
| | | x PD-1 bispecific | KPGQSPQLLIYYASNRYIGVPDRFSGSGSGTDFTLKISR |
| | | antibody) VL | VEAEDVGVYYCQQHYSSPYTFGQGTKVEIK |
| | | polypeptide 4 | |
| 95 | aa | Tiragolumab | EVQLQQSGPGLVKPSQTLSLTCAISGDSVSSNSAAWN |
| | | (MTIG7192A; | WIRQSPSRGLEWLGKTYYRFKWYSDYAVSVKGRITINP |
| | | RG6058) (anti- | DTSKNQFSLQLNSVTPEDTAVFYCTRESTTYDLLAGPF |
| | | TIGIT) Fully heavy | DYWGQGTLVTVSSASTKGPSVFPLAPSSKSTSGGTAA |
| | | Chain | LGCLVKDYFPEPVTVSWNSGALTSGVHTFPAVLQSSG |
| | | | LYSLSSVVTVPSSSLGTQTYICNVNHKPSNTKVDKKVE |
| | | | PKSCDKTHTCPPCPAPELLGGPSVFLFPPKPKDTLMIS |
| | | | RTPEVTCVVVDVSHEDPEVKFNWYVDGVEVHNAKTKP |
| | | | REEQYNSTYRVVSVLTVLHQDWLNGKEYKCKVSNKAL |
| | | | PAPIEKTISKAKGQPREPQVYTLPPSREEMTKNQVSLT |
| | | | CLVKGFYPSDIAVEWESNGQPENNYKTTPPVLDSDGS |
| | | | FFLYSKLTVDKSRWQQGNVFSCSVMHEALHNHYTQKS |
| | | | LSLSPGK |
| 96 | aa | Tiragolumab | DIVMTQSPDSLAVSLGERATINCKSSQTVLYSSNNKKY |
| | | (MTIG7192A; | LAWYQQKPGQPPNLLIYWASTRESGVPDRFSGSGSG |
| | | RG6058) (anti- | TDFTLTISSLQAEDVAVYYCQQYYSTPFTFGPGTKVEIK |
| | | TIGIT) Fully heavy | RTVAAPSVFIFPPSDEQLKSGTASVVCLLNNFYPREAK |
| | | Chain | VQWKVDNALQSGNSQESVTEQDSKDSTYSLSSTLTLS |
| | | | KADYEKHKVYACEVTHQGLSSPVTKSFNRGEC |
| 97 | aa | Tiragolumab | EVQLQQSGPGLVKPSQTLSLTCAISGDSVSSNSAAWN |
| | | (MTIG7192A; | WIRQSPSRGLEWLGKTYYRFKWYSDYAVSVKGRITINP |
| | | RG6058) (anti- | DTSKNQFSLQLNSVTPEDTAVFYCTRESTTYDLLAGPF |
| | | TIGIT) VH | DYWGQGTLVTVSS |
| 98 | aa | Tiragolumab | DIVMTQSPDSLAVSLGERATINCKSSQTVLYSSNNKKY |
| | | (MTIG7192A; | LAWYQQKPGQPPNLLIYWASTRESGVPDRFSGSGSG |
| | | RG6058) (anti- | TDFTLTISSLQAEDVAVYYCQQYYSTPFTFGPGTKVEIK |
| | | TIGIT) VL | |
| 99 | aa | Tiragolumab | GDSVSSNSAA |
| | | (MTIG7192A; | |
| | | RG6058) (anti- | |
| | | TIGIT) HCDR1 | |
| 100 | aa | Tiragolumab | TYYRFKWYS |
| | | (MTIG7192A; | |
| | | RG6058) (anti- | |
| | | TIGIT) HCDR2 | |
| 101 | aa | Tiragolumab | TRESTTYDLLAGPFDY |
| | | (MTIG7192A; | |
| | | RG6058) (anti- | |
| | | TIGIT) HCDR3 | |
| 102 | aa | Tiragolumab | QTVLYSSNNKKY |
| | | (MTIG7192A; | |
| | | RG6058) (anti- | |
| | | TIGIT) LCDR1 | |
| 103 | aa | Tiragolumab | WAS |
| | | (MTIG7192A; | |
| | | RG6058) (anti- | |
| | | TIGIT) LCDR2 | |
| 104 | aa | Tiragolumab | QQYYSTPFT |
| | | (MTIG7192A; | |
| | | RG6058) (anti- | |
| | | TIGIT) LCDR3 | |
| 105 | aa | AZD2936 (anti- | QVQLVESGGGVVQPGRSLRLSCAASGFTFSSYAMHW |
| | | TIGIT/Anti-PD-1 | VRQAPGKGLEWVAVISYAGEVKYYADSVKGRFTISRD |
| | | bispecific antibody) | NSKNTLYLQMNSLRAEDTAVYYCARDPLPLHYYGMDV |
| | | Full Polypeptide 1 | WGQGTTVTVSSASTKGPSVCPLAPSSKSTSGGTAALG |
| | | | CLVKDYFPEPVTVSWNSGALTSGVHTFPAVLQSSGLY |
| | | | SLSSVVTVPSSSLGTQTYICNVNHKPSNTKVDKRVEPK |
| | | | SVDKTHTCPPCPAPEFEGGPSVFLFPPKPKDTLMISRT |
| | | | PEVTCVVVDVSHEDPEVKFNWYVDGVEVHNAKTKPRE |
| | | | EQYNSTYRVVSVLTVLHQDWLNGKEYKCKVSNKALPA |
| | | | SIEKTISKAKGQPREPQVYTLPPCREEMTKNQVSLWCL |
| | | | VKGFYPSDIAVEWESNGQPENNYKTTPPVLDSDGSFF |
| | | | LYSKLTVDKSRWQQGNVFSCSVMHEALHNHYTQKSLS |
| | | | LSPGK |
| 106 | aa | AZD2936 (anti- | DIVMTQSPDSLAVSLGERATINCKSSQTVLYSSNNKKY |
| | | TIGIT/Anti-PD-1 | LAWYQQKPGQPPNLLIYWASTRESGVPDRFSGSGSG |
| | | bispecific antibody) | TDFTLTISSLQAEDVAVYYCQQYYSTPFTFGPGTKVEIK |
| | | Full Polypeptide 2 | RTVAAPSVFIFPPSDEQLKSGTASVVCLLNNFYPREAK |
| | | | VQWKVDNALQSGNSQESVTEQDSKDSTYSLSSTLTLS |
| | | | KADYEKHKVYACEVTHQGLSSPVTKSFNRGEC |
| 107 | aa | AZD2936 (anti- | EVQLVESGGGLVQPGGSLRLSCAASGFTFSDYGMHW |
| | | TIGIT/Anti-PD-1 | VRQAPGKGLEWVAYISSGSYTIYSADSVKGRFTISRDN |
| | | bispecific antibody) | AKNSLYLQMNSLRAEDTAVYYCARRAPNSFYEYYFDY |
| | | Full Polypeptide 3 | WGQGTTVTVSSASTKGPSVFPLAPSSKSTSGGTAALG |
| | | | CLVKDYFPEPVTVSWNSGALTSGVHTFPAVLQSSGLY |
| | | | SLSSVVTVPSSSLGTQTYICNVNHKPSNTKVDKRVEPK |
| | | | SCDKTHTCPPCPAPEFEGGPSVFLFPPKPKDTLMISRT |
| | | | PEVTCVVVDVSHEDPEVKFNWYVDGVEVHNAKTKPRE |
| | | | EQYNSTYRVVSVLTVLHQDWLNGKEYKCKVSNKALPA |
| | | | SIEKTISKAKGQPREPQVCTLPPSREEMTKNQVSLSCA |
| | | | VKGFYPSDIAVEWESNGQPENNYKTTPPVLDSDGSFF |
| | | | LVSKLTVDKSRWQQGNVFSCSVMHEALHNHYTQKSLS |
| | | | LSPGK |
| 108 | aa | AZD2936 (anti- | QIVLTQSPATLSLSPGERATLSCSASSKHTNLYWSRHM |
| | | TIGIT/Anti-PD-1 | YWYQQKPGQAPRLLIYLTSNRATGIPARFSGSGSGTDF |
| | | bispecific antibody) | TLTISSLEPEDFAVYYCQQWSSNPFTFGQGTKLEIKRT |
| | | Full Polypeptide 4 | VAAPSVFIFPPSDEQLKSGTASVVCLLNNFYPREAKVQ |
| | | | WKVDNALQSGNSQESVTEQDSKDSTYSLSSTLTLSKA |
| | | | DYEKHKVYACEVTHQGLSSPVTKSFNRGEC |
| 109 | aa | AZD2936 (anti- | QVQLVESGGGVVQPGRSLRLSCAASGFTFSSYAMHW |
| | | TIGIT/Anti-PD-1 | VRQAPGKGLEWVAVISYAGEVKYYADSVKGRFTISRD |
| | | bispecific antibody) | NSKNTLYLQMNSLRAEDTAVYYCARDPLPLHYYGMDV |
| | | VH Polypeptide 1 | WGQGTTVTVSS |
| 110 | aa | AZD2936 (anti- | QSVLTQPPSASGTPGQRVTISCSGSSSNIGRRPVNWY |
| | | TIGIT/Anti-PD-1 | QQLPGTAPKLLIYSQNQRPSGVPDRFSGSQSGTSASL |
| | | bispecific antibody) | AISGLQSEDEADYFCAVWDDIGRVLQLGGGTQLTVL |
| | | VL Polypeptide 2 | |
| 111 | aa | AZD2936 (anti- | EVQLVESGGGLVQPGGSLRLSCAASGFTFSDYGMHW |
| | | TIGIT/Anti-PD-1 | VRQAPGKGLEWVAYISSGSYTIYSADSVKGRFTISRDN |
| | | bispecific antibody) | AKNSLYLQMNSLRAEDTAVYYCARRAPNSFYEYYFDY |
| | | VH Polypeptide 3 | WGQGTTVTVSS |
| 112 | aa | AZD2936 (anti- | QIVLTQSPATLSLSPGERATLSCSASSKHTNLYWSRHM |
| | | TIGIT/Anti-PD-1 | YWYQQKPGQAPRLLIYLTSNRATGIPARFSGSGSGTDF |
| | | bispecific antibody) | TLTISSLEPEDFAVYYCQQWSSNPFTFGQGTKLEIK |
| | | VL Polypeptide 4 | |
| 113 | aa | AZD2936 (anti- | GFTFSSYA |
| | | TIGIT/Anti-PD-1 | |
| | | bispecific antibody) | |
| | | HCDR1 | |
| | | Polypeptide 1 | |
| 114 | aa | AZD2936 (anti- | ISYAGEVK |
| | | TIGIT/Anti-PD-1 | |
| | | bispecific antibody) | |
| | | HCDR2 | |
| | | Polypeptide 1 | |
| 115 | aa | AZD2936 (anti- | ARDPLPLHYYGMDV |
| | | TIGIT/Anti-PD-1 | |
| | | bispecific antibody) | |
| | | HCDR3 | |
| | | Polypeptide 1 | |
| 116 | aa | AZD2936 (anti- | SSNIGRRP |
| | | TIGIT/Anti-PD-1 | |
| | | bispecific antibody) | |
| | | LCDR1 | |
| | | Polypeptide 2 | |
| 117 | aa | AZD2936 (anti- | SQN |
| | | TIGIT/Anti-PD-1 | |
| | | bispecific antibody) | |
| | | LCDR2 | |
| | | Polypeptide 2 | |
| 118 | aa | AZD2936 (anti- | AVWDDIGRVLQ |
| | | TIGIT/Anti-PD-1 | |
| | | bispecific antibody) | |
| | | LCDR3 | |
| | | Polypeptide 2 | |
| 119 | aa | AZD2936 (anti- | GFTFSDYG |
| | | TIGIT/Anti-PD-1 | |
| | | bispecific antibody) | |
| | | HCDR1 | |
| | | Polypeptide 3 | |
| 120 | aa | AZD2936 (anti- | ISSGSYTI |
| | | TIGIT/Anti-PD-1 | |
| | | bispecific antibody) | |
| | | HCDR2 | |
| | | Polypeptide 3 | |
| 121 | aa | AZD2936 (anti- | ARRAPNSFYEYYFDY |
| | | TIGIT/Anti-PD-1 | |
| | | bispecific antibody) | |
| | | HCDR3 | |
| | | Polypeptide 3 | |
| 122 | aa | AZD2936 (anti- | SKHTNLYWSRH |
| | | TIGIT/Anti-PD-1 | |
| | | bispecific antibody) | |
| | | LCDR1 | |
| | | Polypeptide 4 | |
| 123 | aa | AZD2936 (anti- | LTS |
| | | TIGIT/Anti-PD-1 | |
| | | bispecific antibody) | |
| | | LCDR2 | |
| | | Polypeptide 4 | |
| 124 | aa | AZD2936 (anti- | QQWSSNPFT |
| | | TIGIT/Anti-PD-1 | |
| | | bispecific antibody) | |
| | | LCDR3 | |
| | | Polypeptide 4 | |
| 125 | aa | VH of humanized | QVQLVQSGAEVKKPGASVKVSCKASGYTFTRYYLYWV |
| | | mAb 20.1 | KQRPGQGLEWIGEINPNNGGTKFNEKFKSRATMTVDK |
| | | | STRTTYMELSSLRSEDTAVYYCSREDDYDGTPDAMDY |
| | | | WGQGTLVTVSS |
| 126 | aa | VL of humanized | DIQMTQSPSSLSASVGDRVTITCHASQNINLWLSWYQ |
| | | mAb 20.1 | QKPGKAPKLLIYRASNLHTGVPSRFSGSGSATDFTFTIS |
| | | | SLQPEDIATYYCQQGHSYPYTFGGGTKVDIK |
| 127 | aa | Human LAG-3 | MWEAQFLGLLFLQPLWVAPVKPLQPGAEVPVVWAQE |
| | | | GAPAQLPCSPTIPLQDLSLLRRAGVTWQHQPDSGPPA |
| | | | AAPGHPLAPGPHPAAPSSWGPRPRRYTVLSVGPGGL |
| | | | RSGRLPLQPRVQLDERGRQRGDFSLWLRPARRADAG |
| | | | EYRAAVHLRDRALSCRLRLRLGQASMTASPPGSLRAS |
| | | | DWVILNCSFSRPDRPASVHWFRNRGQGRVPVRESPH |
| | | | HHLAESFLFLPQVSPMDSGPWGCILTYRDGFNVSIMYN |
| | | | LTVLGLEPPTPLTVYAGAGSRVGLPCRLPAGVGTRSFL |
| | | | TAKWTPPGGGPDLLVTGDNGDFTLRLEDVSQAQAGTY |
| | | | TCHIHLQEQQLNATVTLAIITVTPKSFGSPGSLGKLLCE |
| | | | VTPVSGQERFVWSSLDTPSQRSFSGPWLEAQEAQLLS |
| | | | QPWQCQLYQGERLLGAAVYFTELSSPGAQRSGRAPG |
| | | | ALPAGHLLLFLILGVLSLLLLVTGAFGFHLWRRQWRPR |
| | | | RFSALEQGIHPPQAQSKIEELEQEPEPEPEPEPEPEPE |
| | | | PEPEQL |
| 128 | aa | Human TIM-3 | MFSHLPFDCVLLLLLLLLTRSSEVEYRAEVGQNAYLPC |
| | | | FYTPAAPGNLVPVCWGKGACPVFECGNVVLRTDERD |
| | | | VNYWTSRYWLNGDFRKGDVSLTIENVTLADSGIYCCRI |
| | | | QIPGIMNDEKFNLKLVIKPAKVTPAPTRQRDFTAAFPRM |
| | | | LTTRGHGPAETQTLGSLPDINLTQISTLANELRDSRLAN |
| | | | DLRDSGATIRIGIYIGAGICAGLALALIFGALIFKWYSHSK |
| | | | EKIQNLSLISLANLPPSGLANAVAEGIRSEENIYTIEENV |
| | | | YEVEEPNEYYCYVSSRQQPSQPLGCRFAMP |
| 129 | aa | Human TIGIT | MRWCLLLIWAQGLRQAPLASGMMTGTIETTGNISAEK |
| | | | GGSIILQCHLSSTTAQVTQVNWEQQDQLLAICNADLGW |
| | | | HISPSFKDRVAPGPGLGLTLQSLTVNDTGEYFCIYHTYP |
| | | | DGTYTGRIFLEVLESSVAEHGARFQIPLLGAMAATLVVI |
| | | | CTAVIVVVALTRKKKALRIHSVEGDLRRKSAGQEEWSP |
| | | | SAPSPPGSCVQAEAAPAGLCGEQRGEDCAELHDYFN |
| | | | VLSYRSLGNCSFFTETG |
| 130 | aa | Human PD-1 | MQIPQAPWPVVWAVLQLGWRPGWFLDSPDRPWNPP |
| | | | TFSPALLVVTEGDNATFTCSFSNTSESFVLNWYRMSPS |
| | | | NQTDKLAAFPEDRSQPGQDCRFRVTQLPNGRDFHMS |
| | | | VVRARRNDSGTYLCGAISLAPKAQIKESLRAELRVTER |
| | | | RAEVPTAHPSPSPRPAGQFQTLVVGVVGGLLGSLVLL |
| | | | VWVLAVICSRAARGTIGARRTGQPLKEDPSAVPVFSVD |
| | | | YGELDFQWREKTPEPPVPCVPEQTEYATIVFPSGMGT |
| | | | SSPARRGSADGPRSAQPLRPEDGHCSWPL |
| 131 | aa | Human PD-L1 | MRIFAVFIFMTYWHLLNAFTVTVPKDLYVVEYGSNMTIE |
| | | | CKFPVEKQLDLAALIVYWEMEDKNIIQFVHGEEDLKVQ |
| | | | HSSYRQRARLLKDQLSLGNAALQITDVKLQDAGVYRC |
| | | | MISYGGADYKRITVKVNAPYNKINQRILVVDPVTSEHEL |
| | | | TCQAEGYPKAEVIWTSSDHQVLSGKTTTTNSKREEKLF |
| | | | NVTSTLRINTTTNEIFYCTFRRLDPEENHTAELVIPELPL |
| | | | AHPPNERTHLVILGAILLCLGVALTFIFRLRKGRMMDVK |
| | | | KCGIQDTNSKKQSDTHLEET |
| 132 | aa | Tebotelimab- VL | QVQLVQSGAEVKKPGASVKVSCKASGYSFTSYWMNW |
| | | Polypeptide 4 | VRQAPGQGLEWIGVIHPSDSETWLDQKFKDRVTITVDK |
| | | | STSTAYMELSSLRSEDTAVYYCAREHYGTSPFAYWGQ |
| | | | GTLVTVSS |

### EXAMPLES

### 1. Methods to Characterize BTN3A Activating Antibodies for Use According to the Present Disclosure

### 1.1 Binding Affinity Assay: Multi-Cycle Kinetic Assay (SPR)

Multi-cycle kinetic analysis can be performed with BTN3A antibodies using a Biacore T200 (serial no. 1909913) instrument running Biacore T200 Evaluation Software V2.0.1 (Uppsala, Sweden).

Purified antibodies are diluted to a concentration of 2 μg/ml in 2% BSA/PBS. At the start of each cycle, each antibody is captured on the Protein A at a density (RL) of ~146.5 RU (theoretical value to obtain an RMax of ~50 RU). Following capture, the surface is allowed to stabilize before injection of the BTN3A1 antigen (Sino Biological cat. no. 15973-H08H). BTN3A1 is titrated in 0.1% BSA/HBS-P+ (running buffer) in a two-fold dilution range from 25 to 0.78 nM. The association phase is monitored for 400 seconds and the dissociation phase for 35 minutes (2100 seconds). Kinetic data is obtained using a flow rate of 50 μl/min to minimize any potential mass transfer effects. Regeneration of the Protein A surface is conducted using two injections of 10 mM glycine-HCL pH 1.5 at the end of each cycle. Two blanks (no BTN3A1) and a repeat of a single concentration of the analyte are performed for each tested antibody to check the stability of the surface and analyte over the kinetic cycles. The signal from the reference channel Fc1 is subtracted from that of Fc2, Fc3 and Fc4 to correct for differences in non-specific binding to a reference surface. Additionally, blank runs are subtracted for each Fc to correct any antigen-independent signal variation, such as drift. Sensorgrams are fitted using a one-to-one binding mathematical model with a global RMax parameter and no bulk signal (Constant RI=0 RU).

### 1.2 Binding Assay by Flow Cytometry on Human PBMCs

BTN3A activating antibodies for use according to the present disclosure may also be characterized for their binding to human PBMCs, isolated from blood of healthy donors. PBMCs are isolated from buffy coats using Lymphoprep (Axis-shield, Dundee, UK) density centrifugation. PBMCs are then frozen and stored at −80° C. or in liquid nitrogen until required.

100 μl cells at 1×10⁶ cells/ml are transferred to each well of a fresh U-shaped bottom 96-well plate, then the plate was centrifuged and supernatant discarded.

A serial dilution of the antibodies, 0.001 μg/ml to 150 μg/ml is prepared in PBS 2 mM EDTA. Human PBMCs were resuspended in 50 μl of the diluted test antibody titration series prepared.

After incubation for 30 minutes at 4° C. in the dark, the plate was centrifuged and washed twice with 150 μl/well of PBS 2 mM EDTA following which the wells are resuspended in 50 μl of a mix composed of goat anti-human antibody (PE labelled) diluted 1/100 and Live/dead near IR diluted 1/500 in PBS 2 mM EDTA.

After incubation for 15 minutes at 4° C. in the dark, the plate is centrifuged and washed once with 150 μl/well PBS 2 mM EDTA following which the wells are resuspended in 200 μl PBS 2 mM EDTA. Cells are analyzed on a BD LSR Fortessa Cytometer. Data is analyzed using a FlowJo software (Version 10, FlowJo, LLC, Ashland, USA).

Same protocol may be performed on cynomolgus PBMCs and on Daudi Burkitt's lymphoma cell line.

### 1.3 In Vitro Functional Efficacy: γδ-T Cell Degranulation Assay

The assay consists of measuring activating or inhibitory effect of BTN3A antibodies on γδ-T cell degranulation against Daudi Burkitt's lymphoma cell line (Harly et al., 2012). γδ-T cells are expanded from PBMCs of healthy donors by culturing with zoledronic acid (1 μM) and IL2 (200 IU/ml) for 11-13 days. IL2 is added at day 5, day 8 and every 2 days thereafter. The percentage of γδ-T cells is determined at the initiation of culture and assessed for the time of culture by flow cytometry until it reached at least 80%. Frozen or fresh γδ-T cells are then used in degranulation assays against Daudi cell line (E:T ratio of 1:1), whereby the cells are co-cultured for 4 hours at 37° C. in presence of 10 μg/ml of the 7.2 and/or 20.1 humanized variants and/or their chimeric versions. Activation by PMA (20 ng/ml) plus lonomycin (1 μg/ml) serves as positive control for γδ-T cell degranulation, and medium alone as negative control. At the end of 4 hours co-incubation, cells are analyzed by flow cytometry to evaluate the percentage of γδ-T cells positive for CD107a (LAMP-1, lysosomal-associated membrane protein-1)+CD107b (LAMP-2). CD107 is mobilized to the cell surface following activation-induced granule exocytosis, thus measurement of surface CD107 is a sensitive marker for identifying recently degranulated cytolytic T cells.

The same protocol may be performed using AML blasts isolated from patients as target cells, in place of Daudi cells.

### 1.4 In Vitro Functional Efficacy: Vγ9Vδ2 T Cell Activation in PBMC

The assay consists of measuring the activating effect of BTN3A antibodies on Vγ9Vδ2 T cells in PBMC. Human PBMCs were isolated by Ficoll density gradient centrifugation of peripheral blood (EDTA-buffy coats or heparinized whole blood). When using whole blood, RBC were depleted using 1×RBC Lysis Buffer (eBioscience) for 10 minutes at room temperature and then washed with PBS 1% FBS.

PBMCs or RBC-depleted cells were cultured in RPMI 1640 10% FBS, 1% P/S at 1.5 to 3×10⁶ cells/mL, with increasing concentrations of anti-BTN3A antibodies (dose range 0.00001 to 100 μg/mL) at 37° C., 5% CO₂ in a volume of 200 μL in 96 round-bottom well plates. Activation status was monitored after two days of culture by flow cytometry analysis of activation marker surface expression. Cells were washed in PBS 2% FBS 2 mM EDTA (FACS buffer). Cells were centrifuged at 1800 rpm for 5 minutes and then incubated 10 minutes at room temperature with 10 μl of FcR blocking Reagent (Miltenyi Biotec) before adding 30 to 50 μL of the appropriate antibody mix prepared in FACS buffer and containing at least fluorescently-conjugated anti-CD3, anti-Vg9 or -Vd2 TCR and anti-CD69 antibodies. A viability marker (LIVE/DEAD Fixable Dead Cell Stain) was added in all experiments in order to exclude dead cells from the analysis. Cells were incubated 30 minutes at 4° C. and washed 2 times in FACS buffer before fixation in Cytofix Fixation Buffer (BD Bioscience) and flow cytometry analysis. Data were analyzed with flowjo V-10.6 software. Activated Vγ9Vδ2 T cells were defined as CD3+Vd2+ (or Vg9+ or Vd2+Vg9+) CD69+.

### 2. Materials and Methods of the Experimental Part

### 2.1 PBMC Isolation and Vγ9Vδ2 T Cell Expansion

Human ethylenediaminetetraacetic acid (EDTA)-buffy coats were obtained from the Etablissement Français du Sang (EFS) Provence Alpes Côthe d'Azur (France) from healthy donors (HD). Peripheral blood mononuclear cells (PBMC) were isolated by Ficoll density gradient centrifugation of EDTA-buffy coats.

To expand γδ T cells for killing assay, healthy donors human PBMC (HD Hu-PBMC) were resuspended in complete medium (RPMI 1640 medium supplemented with 10% FBS, 1% sodium pyruvate and 1% P/S) for 12-13 days in the presence of recombinant human Interleukin-2 (rhIL-2 (ProLeukin®, Novartis) (200 International Units (IU)/mL) and aminobisphosphonates (Zoledronate (Sigma-Aldrich #SML0223), 1 μM). From day 5, rhIL-2 was replenished every 2 or 3 days by adding fresh complete culture medium supplemented with rhIL-2 and cells were kept at a concentration of 1×10⁶/mL. Expanded cells were collected at day 12-13 for phenotype and killing assay assessment.

### 2.2 Flow Cytometry Assessment for Surface Expression of Markers of Interest on Effector T Cells and Tumor Cell Lines

For assessment of LAG-3, TIM-3 and TIGIT expression on unstimulated and ICT01-stimulated Vγ9Vδ2 T cells, fresh Hu-PBMC isolated from healthy donors (HD) were resuspended in complete medium and cultured for 5 days in the presence of 1 μg/mL of ICT01 or its corresponding isotype control (hIgG1S) with or without 50 IU/ml of rhIL-2 (ProLeukin®). Before culture and at indicated times after incubation, cells were harvested for assessment of LAG-3, TIM-3 and TIGIT surface expression by flow cytometry performed on a Cytoflex LX instrument (Beckman Coulter). Briefly, cells were washed with PBS supplemented with 2% FBS and 2 mM EDTA (FACS buffer), then incubated 10 minutes at room temperature with FcR blocking Reagent (Miltenyi Biotec) before staining with a mix of conjugated mAbs (anti-CD14 APC Vio770, anti-CD19 BV650, anti-CD3 Alexa fluor 700, anti-Vd2TCR FITC, anti-TIM-3-BV785, anti-TIGIT PE-cy7 and anti-LAG-3 APC) and viability marker (LIVE/DEAD™ near IR) for 20 minutes at 4° C. Cells unstained for the markers of interest (TIM-3, LAG-3 and TIGIT) were used in parallel as control. After incubation, stained cells were washed twice in FACS buffer before acquisition.

In vitro expanded Vγ9Vδ2 T cells were analysed for LAG-3, TIM-3 and TIGIT and MHC class II surface expression by flow cytometry using conjugated mAbs (anti-CD3 Alexa fluor 700, anti-Vd2 TCR FITC, anti-LAG-3 APC, anti-TIGIT PE-cy7, anti-TIM-3 PE Dazzle594 and anti-MHC class II PE) and a viability marker (LIVE/DEAD™ near IR) using the same staining procedure as described above. A second mix of mAbs containing the respective isotype controls for anti-TIM-3, anti-LAG-3, anti-TIGIT and anti-MHC class II was done in parallel to allow proper gating.

Assessment of BTN3A and ligands of LAG-3 (MHC class II), TIM-3 (Galectin 9) and TIGIT (PVR and Nectin 2) expression on tumor target cell lines was performed by flow cytometry using conjugated Abs (anti-MHC class II PE, anti-PVR PV421, anti-BTN3A AF546), purified mAbs (anti-Nectin-2, anti-Galectin-9) and a viability marker (LIVE/DEAD™ near IR) using the same staining procedure as described above. For staining with purified Ab, a second step of staining with a labelled secondary Ab was performed. An additional antibody cocktail containing the respective isotype control of markers of interest were used in parallel to allow proper gating.

Assessment of LAG-3, TIM-3 and TIGIT expression on circulating Vγ9Vδ2 T cells of cancer patients enrolled in the EVICTION trial (as detailed in table below) at baseline and following treatment by different doses of ICT01 plus pembrolizumab was performed by flow cytometry analysis on frozen PBMC. After thawing, PBMC were counted and 1×10⁶ cells were mixed with an antibody cocktail containing anti-CD14 APC Vio770, anti-CD3 Alexa fluor 700, anti-Vd2TCR PE, anti-TIM-3 BB515, anti-TIGIT PE-Cy7 and anti-LAG-3 PE-Dazzle594 and viability marker (LIVE/DEAD™ near IR). An additional antibody cocktail containing the respective isotype controls of anti-TIM-3, anti-LAG-3 and anti-TIGIT were used in parallel.

List of cancer patients (EVICTION trial)

| Group | ICT01 dose | Number |
|---|---|---|
| C1 | 700 μg | n = 4 (Non-small Cell Lung Cancer (NSCLC) n = 2; Bladder |
| | | n = 1; urothelial carcinoma n = 1) |
| C2 | 2 mg | n = 7 (NSCLC n = 3; Melanoma n = 3; Bladder n = 1) |
| C3 | 7 mg | n = 6 (Non-small Cell Lung Cancer (NSCLC) n = 2; Bladder |
| | | n = 1; urothelial carcinoma n = 2; Melanoma n = 1) |
| C4 | 20 mg | n = 6 (NSCLC n = 4; Melanoma n = 2) |
| C5 | 75 mg | n = 7 (Lung carcinoma n = 1; Melanoma n = 2; NSCLC n = 1; |
| | | Head and neck squamous cell carcinoma (HNSCC) n = 1; |
| | | urothelial carcinoma n = 2) |
| C6 | 200 mg | n = 5 (NSCLC n = 2; urothelial carcinoma n = 1; Melanoma n = 2) |

Assessment of PD-1, LAG-3, TIM-3 and TIGIT expression on tumor infiltrating Vγ9Vδ2 T cells and CD8+ T cells of cancer patients (as detailed in table below) was performed by flow cytometry analysis on fresh and frozen dissociated tumor cells. Fresh dissociated tumor cells were isolated from fresh tumors by mechanic dissociation using gentleMACS™ Octo Dissociator (Miltenyi Biotec). Briefly, fresh and frozen dissociated tumor cells were washed with PBS supplemented with 2% FBS and 2 mM EDTA (FACS buffer), then incubated 10 minutes at room temperature with FcR blocking Reagent (Miltenyi Biotec) before staining with a mix of conjugated mAbs (anti-CD14 BUV805, anti-CD19 BUV496, anti-CD3 RedFluor710, anti-Vd2TCR PE, anti CD8 BUV395, anti CD4 BUV563, anti-PD-1 BV421, anti-LAG-3 PE-Dazzle594; anti-TIM-3 BB515 and anti-TIGIT PE-Cy7) and a viability marker (LIVE/DEAD™ near IR) for 20 minutes at 4° C. Each sample was also stained with a second mix of mAbs containing the respective isotype controls for anti-PD-1, anti-LAG-3, anti-TIM-3 and anti-TIGIT to allow proper gating. After incubation, stained cells were washed twice in FACS buffer before acquisition on a Cytek® Aurora spectral flow cytometer.

List of tumor samples used for PD-1, LAG-3, TIM-3
and TIGIT expression assessment

| | | Sample | |
|---|---|---|---|
| Indication | Sample type | number | Supplier |
| Bladder cancer | Frozen | 7 | Discovery |
| | dissociated | | Life Sciences |
| | cells | | |
| | Fresh tumor | 9 | BioIVT |
| Head and neck cancer | Fresh tumor | 9 | BioIVT |
| Melanoma | Fresh tumor | 4 | BioIVT |

2.3 BrightPlex® IHC Staining TIM-3, PD-1, LAG-3, CD8, CD3 on FFPE Tissue from Cancer Patients Enrolled in EVICTION Trial

Quantification of intratumoral CD8+ T cells expressing PD-1, LAG-3 and TIM-3 was performed by immunohistochemistry (IHC) analysis performed on FFPE biopsies of cancer patients enrolled in the EVICTION trial (as detailed in table below) before and after treatment (at day 28) with ICT01 in monotherapy or in combination with Pembrolizumab, using Veracyte BrightPlex® technology. Briefly, sequential IHC staining are performed using anti-PD-1 (Origene), anti-TIM-3 (Ozyme), anti-LAG-3 (Ozyme), anti CD3 (Veracyte) and anti CD8 (Veracyte) on a single 4 μm FFPE tissue slide. Successive staining is performed on a Leica Bond RX using MACH 2 polymer detection (Biocare), AMEC chromogen (Vector Lab) and counterstaining using Hematoxylin is then performed at the end of each staining. Coversliping is performed manually with Histodenz mounting medium to protect the stained section. Slides are let drying for a minimum of 15 minutes. Visual quality control is then performed to eliminate bubbles if needed and the slides are then cleaned to optimize efficiency of the scanning step. Each slide is scanned with the Nanozoomer XR×20/Nanozoomer S360×20. Coverslips are removed from slides using warm water bath. Slides are then AMEC destained by ethanol and staining antibody are re stripped with denaturing solution and incubation in PT LINK. The digital pathology allows the quantification of positive cells into whole tumor area, tumor cells area and stroma area. Each sample is analyzed using Veracyte Digital Pathology Platform on Halo software.

Tumor Biopsies from patients enrolled in the EVICTION trial and used to
assess PD-1, LAG-3 and TIM-3 expression on infiltrating CD8 T cells.

| | | Number of pairs |
|---|---|---|
| | | (i.e patients with |
| | | pre-and post-treatment |
| | | biopsies available) |
| Group | Indication | (Total = 16) |
| A | Melanoma | 1 |
| (ICT01 in Monotherapy) | Breast cancer | 1 |
| | Ovarian cancer | 1 |
| | Prostate cancer | 2 |
| | Colorectal cancer | 4 |
| C | Melanoma | 6 |
| (ICT01 plus Pembrolizumab) | NSCLC | 1 |

### 2.4 Vγ9Vδ2 T Cell Activation and Proliferation in HD Hu-PBMC by Flow Cytometry

Fresh HD Hu-PBMC from healthy donors were stained with Cell Trace Violet (CTV) reagent (Invitrogen) and cultured in RPMI 1640 medium supplemented with 10% FBS, 1% P/S at 2.5×10⁶ cells/mL in presence or not of 0.1 μg/mL of ICT01 and 50 IU/mL of rhIL-2 (ProLeukin®) for 3 and 6 days at 37° C., 5% CO₂. 10 μg/mL of blocking anti-TIM-3 (F38-2E2 clone, Biolegend) or anti-TIGIT (MBSA43 clone, ThermoFisher Scientific) or anti-LAG-3 (17B4 clone, Adipogen) versus corresponding control isotype (mIgG1, Miltenyi) or LAG-3 Fc (produced in CHO, Adipogen) versus corresponding control (Ig Fc, produced in CHO, Adipogen) were added in ICT01-activated HD Hu-PBMC at beginning of culture. At indicated timepoints, cells were harvested and stained with conjugated Abs prepared in FACS buffer (anti-CD3 Alexa fluor 700, anti-Vg9 TCR FITC, anti-Vd2 TCR PE, anti-CD25 APC LIVE/DEAD™ near IR) for evaluation of the frequency, activation, and proliferation of Vγ9Vδ2 T cells using Flow cytometry performed on a Cytoflex LX instrument (Beckman Coulter). For γδ T cell expansion assessment, Count Bright Absolute Counting Beads (ThermoFisher Scientific) were added in FACs buffer solution before acquisition to calculate relative cell numbers at day 6 according to manufacturer's instructions.

For evaluating the potential synergistic interaction of LAG-3 Fc and ICT01 on Vγ9Vδ2 T cell activation, fresh HD Hu-PBMC were incubated with or without ICT01 used at 0.03 and 0.1 ug/mL in RPMI 1640 medium supplemented with 10% FBS, 1% P/S at 2.5×10⁶ cells/mL and 50 IU/mL of rhIL-2 (ProLeukin®) for 2 days. LAG-3 Fc or Ig Fc (Adipogen) were added at different concentrations ranging from 0.16 to 10 μg/mL at beginning of the culture. After 2 days of incubation, cell-free culture supernatants were collected and stored for further analysis while cells were harvested and stained with conjugated Abs prepared in FACS buffer (anti-CD3 BV786, anti CD14 APC Vio770, anti CD56 PE Vio770, anti-Vd2 TCR FITC, anti-CD8 Alexa Fluor 700, anti-CD11b BV650, anti-CD25 APC, anti-CD86 PE and LIVE/DEAD™ aqua) for evaluation of the frequency and activation of Vγ9Vδ2 T cells by flow cytometry performed on a Cytoflex LX instrument (Beckman Coulter).

For evaluating the direct effect of LAG-3 Fc on the activation of MHC class II high Vγ9δ2 T cells, fresh HD Hu-PBMC were incubated with or without ICT01 used at 0.1 μg/mL in RPMI 1640 medium supplemented with 10% FBS, 1% P/S at 2.5×10⁶ cells/mL for 2 days. After 2 days of incubation, total CD3+ T cells were sorted from either untreated or ICT01-pretreated PBMC by negative selection using EasySep Human T cell enrichment Kit (STEMCELL technologies). Sorted T cells were first assessed for CD25 and MHC class II expressions on Vγ9δ2 T cells by incubating 1·10⁵ of sorted T cells with conjugated Abs prepared in FACS buffer (anti-CD3 BV786, anti CD11b BV650, anti-CD8 Alexa Fluor 700, anti-Vd2TCR FITC, anti-CD25 APC, anti-MHC II PE) for flow cytometry analysis on a Cytoflex LX instrument. Sorted T cells were then incubated with LAG-3 Fc or Ig Fc control used at 1 μg/mL in RPMI 1640 medium supplemented with 10% FBS, 1% P/S at 2.5×10⁶ cells/mL and 50 IU/mL of rhIL-2 (ProLeukin®) for one day. After 24 hours of incubation, cells were harvested and stained with conjugated Abs prepared in FACS buffer (anti-CD3 Alexa Fluor 700, anti-Vg9 TCR FITC, anti-Vd2 TCR PE, anti-CD25 APC, anti-CD69 PE Cy7 and LIVE/DEAD™ near IR) for evaluation of the activation of Vγ9Vδ2 T cells by flow cytometry performed on a Cytoflex LX instrument (Beckman Coulter).

### 2.5 Cytokine Secretion Assessment by ELISA and Luminex

Supernatants from a 3 day-culture of unstimulated and ICT01-stimulated HD Hu-PBMC treated with blocking mAbs were collected and analyzed for IFN-γ and Granzyme B amount by ThermoFisher Scientific using Luminex technology.

Supernatants from a 3 day-culture of unstimulated and ICT01-stimulated HD Hu-PBMC treated with a single dose of LAG-3 Fc or Ig Fc protein were collected and analyzed for IFN-γ and Granzyme B using a human IFN-γ uncoated ELISA kit (ThermoFisher Scientific) and a human Granzyme B Duoset ELISA (R&D Biotech) respectively according to manufacturer's instructions.

The IFN-γ and TNF-α quantification in a 2 day-culture of unstimulated and ICT01-stimulated HD Hu-PBMC treated with different doses of LAG-3 Fc or Ig Fc protein were performed by ThermoFisher Scientific using Luminex technology.

2.6 Coculture of Expanded Vγ9Vδ2 T Cells or HD Hu-PBMC with Cancer Cell Lines (Ovarian Cancer Origin (SKOV-3) or Leukemic Cell Line (THP1))

SKOV-3 cell line (human Ovarian adenocarcinoma) was purchased from the European Collection of Authenticated Cell Cultures (ECACC) and cultured at 37° C. and 5% CO₂ in McCoy's glutamax medium supplemented with 10% FBS and 1 nM Sodium Pyruvate. mKate-expressing SKOV3 cells were established by lentiviral transduction with the IncuCyte® NucLight Red Lentivirus Reagent (Sartorius) at a multiplicity of infection of 3 in the presence of 8 μg/ml of Polybrene® (Sigma-Aldrich). Selection of transduced cells was performed by adding 1 μg/mL puromycin (Invivogen) to the culture. Cells were tested for mycoplasma contamination by PCR using the MycoplasmaCheck platform (Eurofins).

THP1 cell line (human acute monocytic leukemia) was purchased from ATCC and cultured at 37° C. and 5% CO₂ in RPMI Glutamax 10% FBS 1 mM Sodium Pyruvate 2.5 g/L D-glucose 0.05 mM b-mercaptoethanol 10 mM HEPES. Cells were tested for mycoplasma contamination by PCR using the MycoplasmaCheck platform (Eurofins).

For degranulation assay against tumors cells, expanded Vγ9Vδ2 T cells were cultured for 4 hours with THP1 or overnight with SKOV3 at 1:1 ratio in presence of a mix of anti-CD107a PE and anti-CD107b PE mAbs with or without ICT01 used at 1 or 0.1 μg/mL. LAG-3 Fc or corresponding control (Ig Fc) were added at 1 μg/mL. After coculture, cells were washed and incubated for 10 minutes in FcR blocking reagent prior to staining with a mix of conjugated mAb (anti-CD3 Alexa Fluor 700, anti-Vd2 TCR FITC and viability marker (LIVE/DEAD™ near IR) for 20 minutes at 4° C. Stained cells were extensively washed before acquisition on a Cytoflex S instrument (Beckman coulter).

For expanded Vγ9Vδ2 T cells-mediated killing of THP1 and SKOV3, in vitro expanded Vγ9Vδ2 T cells were preincubated with 10 ug/mL of blocking mAbs specific for TIM-3 (F38-2E2 clone, Biolegend) or TIGIT (MBSA43 clone, ThermoFisher Scientific) or LAG-3 (17B4 clone, Adipogen) or corresponding control isotype (mIgG1; Miltenyi) for 30 minutes and then cocultured for 4 hours with Cell trace violet (CTV)-labeled THP1 or overnight with CTV-labeled SKOV3 at 1:1 ratio. After culture, cells were centrifuged and incubated for 10 minutes in FcR blocking Reagent prior to staining with a mix of anti-CD3 Alexa Fluor 700, anti-Vd2 TCR FITC mAbs and viability marker (LIVE/DEAD™ Fixable) for 20 minutes at 4° C. After incubation, cells were washed twice in FACS buffer and annexin V binding buffer before staining with annexin V-APC reagent prepared in the appropriate buffer for 20 minutes at room temperature. After incubation, cells were analysed within 4 hours post-staining by flow cytometry.

For HD Hu-PBMC-mediated killing of SKOV3, 2.5×10³ of mkate expressing SKOV3 were seeded in 96 wells plate and cultured overnight in appropriate complete medium. The day of the experiment, 12.5×10³ of fresh HD hu-PBMC were added and cultured for 120 hours in presence of single agents (0.1 ug/mL of ICT01 or 10 ug/mL of anti-LAG-3 (Relatlimab, Selleckchem) or anti-TIM-3 (Sabatolimab, Selleckchem) versus corresponding isotype (hIgG4), or the combinations of ICT01 plus anti-LAG-3 or ICT01 plus anti-TIM-3. Wells containing target cells only were used as controls of tumor cell growth and viability. The total number of tumor target cells was followed by the fluorescent signal emitted by the NucLight red dye using the Incucyte® live cell imaging system. Tumor cell growth is presented as the tumor cell count over time normalized to baseline (the tumor cell count 3 hours after the start of the co-culture).

### 2.7 Statistics

Two-way ANOVA and Holm-Sidak's multiple comparisons test were used to determine the effect of two independent variables on a continuous dependent variable. Wilcoxon tests were used to compare two paired groups. Statistics were computed using GraphPad Prism 8.0 (GraphPad Software). Differences were considered significant when the P value was less than 0.05. *(p<0.05), **(p<0.01) ***(p<0.001) and ****(p<0.0001)

### 3. Results of the Experimental Part

3.1 ICT01 Increases LAG-3, TIM-3 and TIGIT Expression In Vitro and on Circulating Vγ982 T Cells from Cancer Patients

LAG-3, TIM-3 and TIGIT expression on Vγ9Vδ2 T cells were assessed at steady state and following ICT01 treatment by incubating hu-PBMC from healthy donors (HD) with ICT01 or its corresponding isotype (hIgG1s).

As shown in FIG. 1A, LAG-3, TIM-3 and TIGIT were weakly detected on Vγ9Vδ2 T cells at steady state but rapidly upregulated following ICT01+ rhIL-2 treatment as compared to cells treated with hIgG1S+rhIL-2. A significant difference was observed over time between the ICT01 and hIgG1S treated samples for LAG-3, TIM-3 and TIGIT expression on Vγ9Vδ2 T cells with LAG-3 and TIM-3 being particularly high at day 5 in presence of ICT01. Similar results were obtained with cell cultures without rhIL-2 (data not shown).

Then, LAG-3, TIM-3 and TIGIT expression were monitored on circulating Vγ9Vδ2 T cells in PBMC of cancer patients from the EVICTION clinical trial (NCT04243499), a first-in-human, two-part, open-label, clinical study designed to assess the safety, tolerability and activity of intravenous doses of ICT01 as monotherapy and in combination with pembrolizumab in patients with advanced-stage, relapsed/refractory cancer (sample detailed in Material & Method section). A strong heterogeneity was observed for the expression of these molecules on circulating cancer patients' Vγ9Vδ2 T cells before treatment initiation. Importantly, the frequency of TIM3+, LAG3+ and TIGIT+Vγ9Vδ2 T cells was increased in most patients treated by ICT01 in combination with Pembrolizumab, with a significant increase at day 7 post dosing (FIG. 1B).

### 3.2 LAG-3, TIM-3 and TIGIT are Expressed on Tumor Infiltrating Vγ9Vδ2 T Cells

The assessment of immune checkpoint receptor expression, encompassing LAG-3, TIM-3, TIGIT, and PD-1, was conducted on tumor-infiltrating T cells obtained from dissociated tumors of bladder cancer patients, head and neck cancer, and melanoma. The expression of these receptors on infiltrating Vγ9Vδ2 T cells was highly variable across samples, with overall frequency of PD1+Vγ9Vδ2 T cells ranging from 0 to 92% (Median 29.2% SEM 3.6), overall frequency of TIM3+Vγ9Vδ2 T cells ranging from 0 to 68% (Median 11.7% SEM 3.4), overall frequency of TIGIT+Vγ9Vδ2 T cells ranging from 1.1 to 67.3% (Median 18.9% SEM 3.4), and LAG-3 being less prevalent with overall frequency of LAG-3+Vγ9Vδ2 T cells ranging from 0 to 13.5% (Median 2.4% SEM 0.76) (FIGS. 2A and B). In addition to Vγ9Vδ2 T cells, elevated levels of these immune checkpoint receptors were observed on infiltrating CD8+ T cells, in particular TIM-3 and PD-1 (FIGS. 2A and B).

3.3 Intratumoral PD-1+TIM-3+LAG-3+CD8+ T Cells are Increased in Patients Treated with ICT01

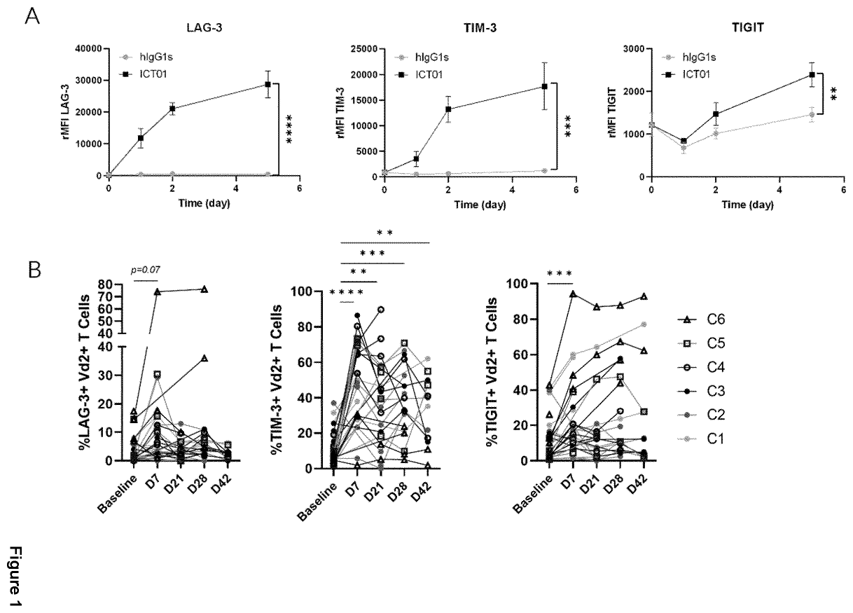

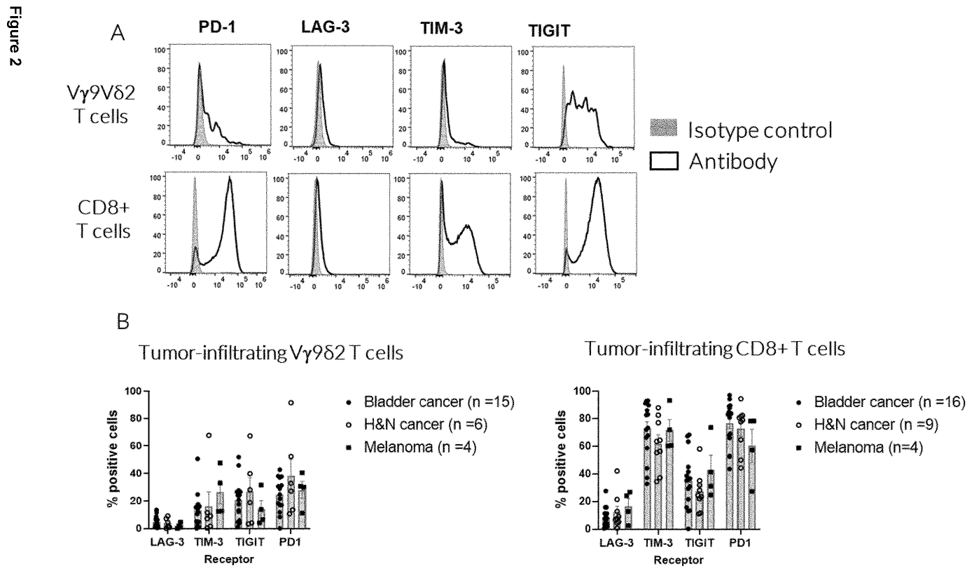

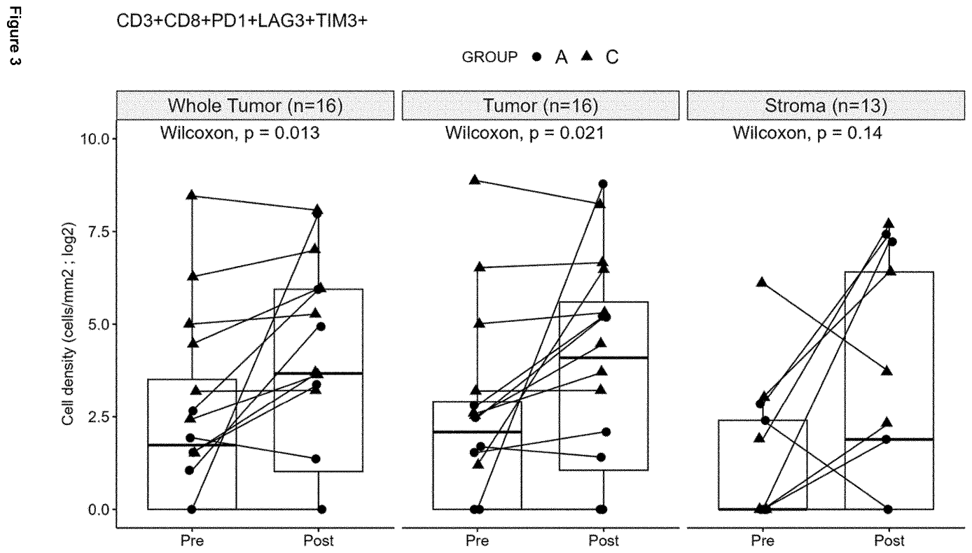

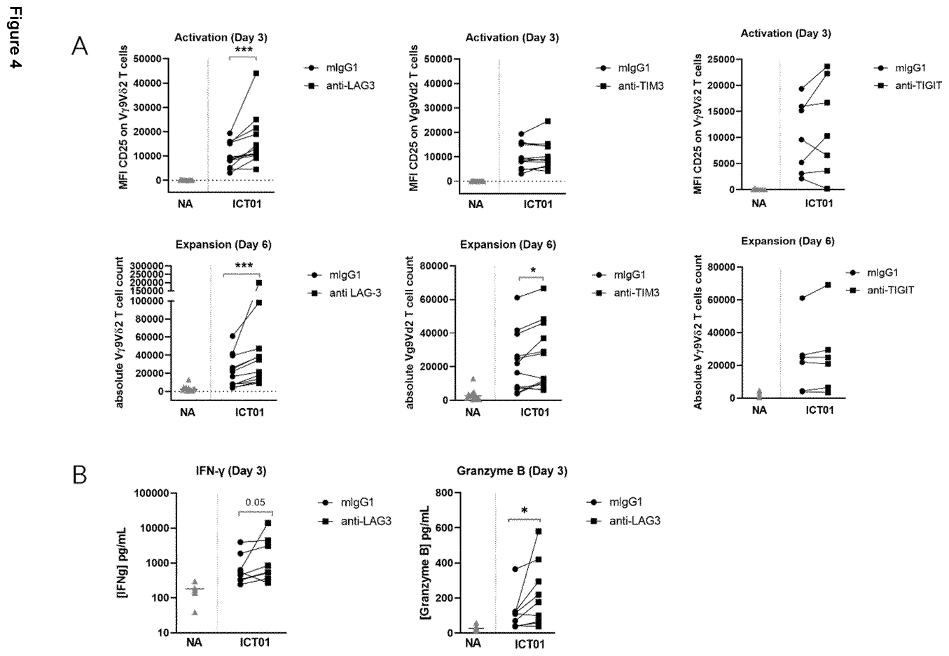

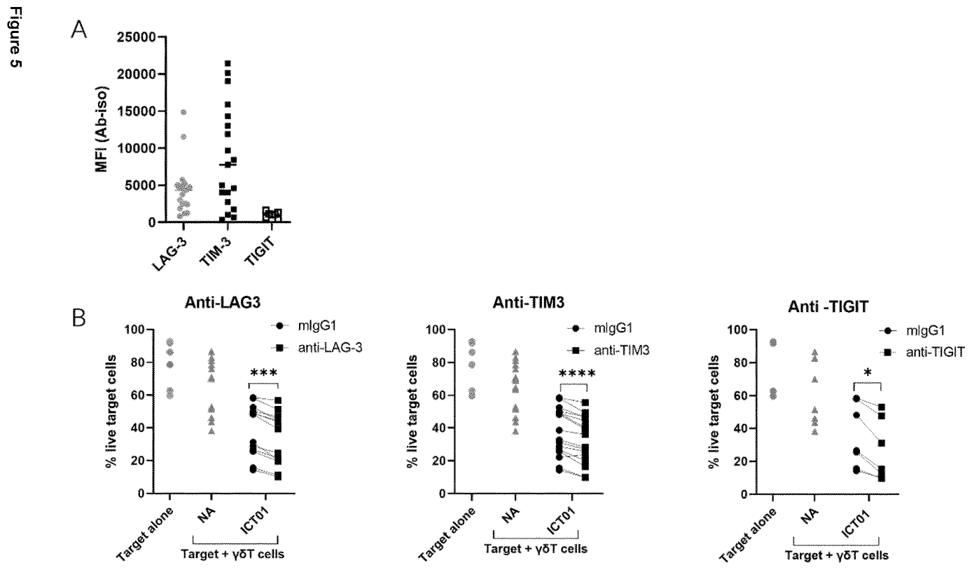

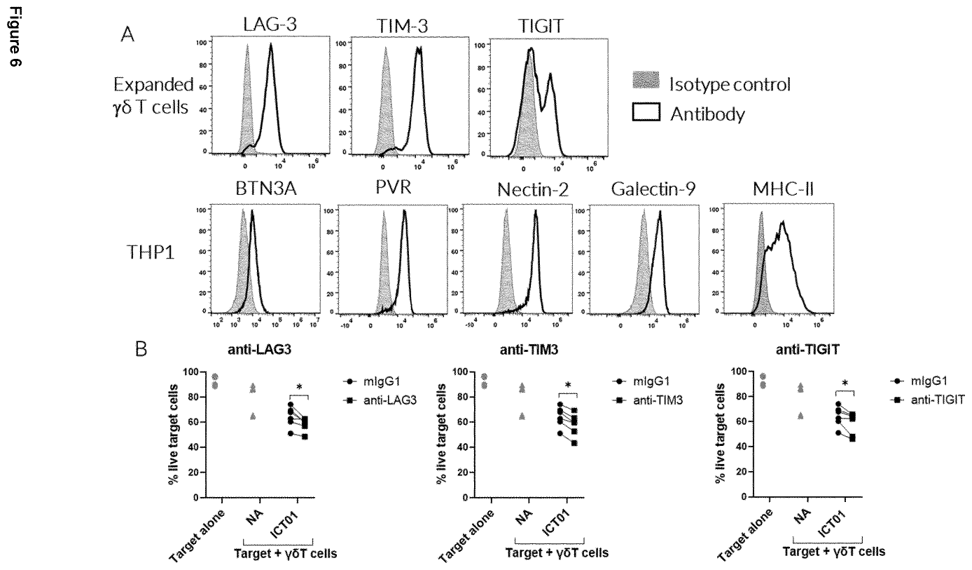

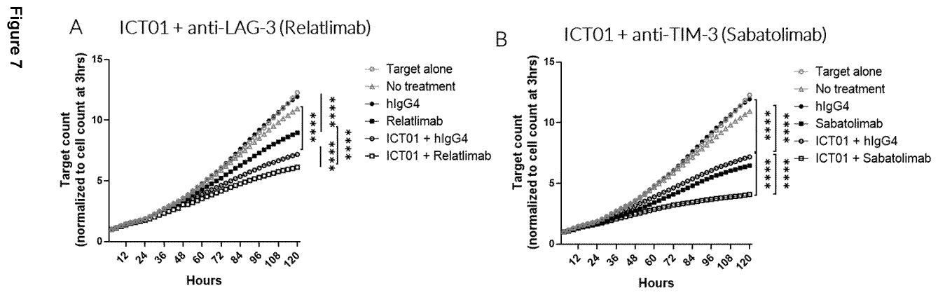

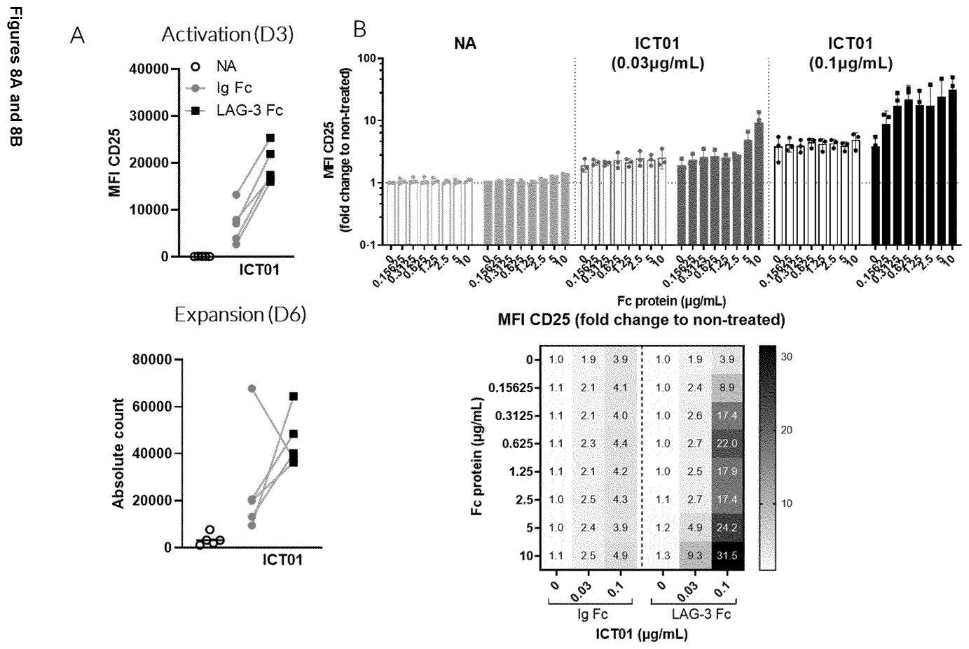

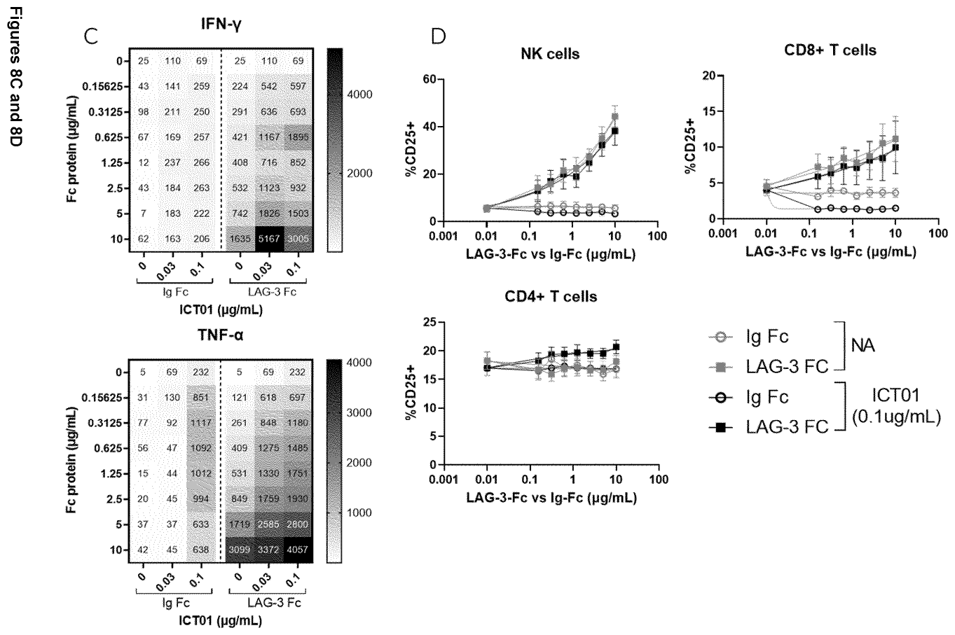

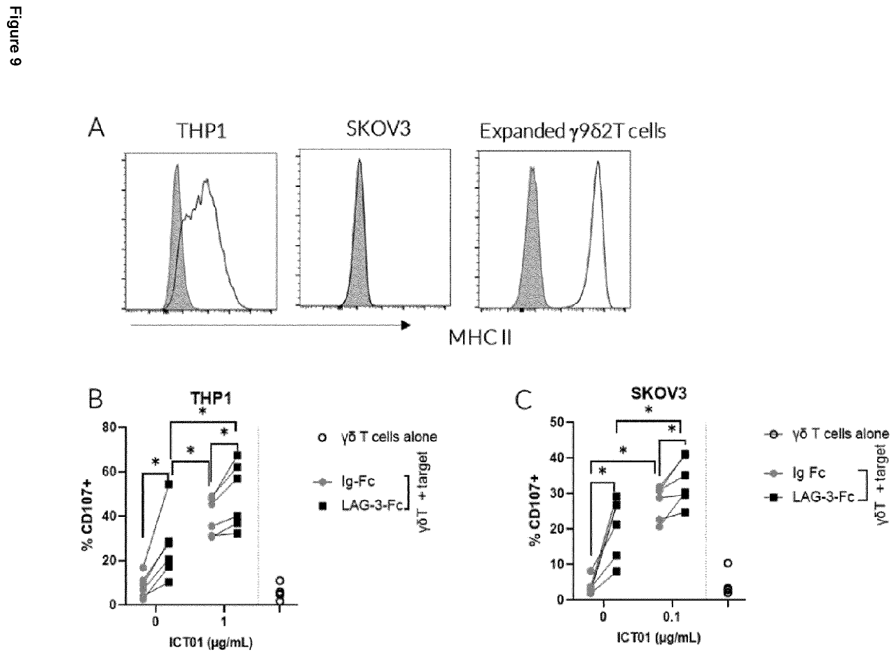

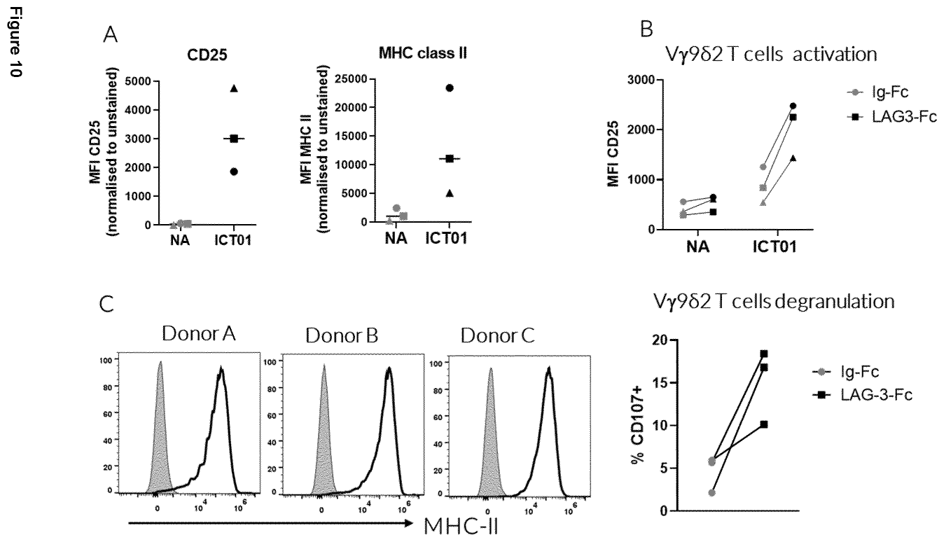

We investigated the impact of ICT01 treatment on the expression of PD-1, TIM-3, and LAG-3 on intratumoral CD8+ T cell in cancer patients enrolled in the EVICTION clinical trial (NCT04243499). Tumor biopsies were collected at baseline and on Day 28 (7 days after the second dose of ICT01) and PD-1, TIM-3, and LAG-3 were stained together with CD3 and CD8 specific antibodies using multiplex IHC method. FIG. 3 depicts a significant increase in CD8 T cells coexpressing the immune checkpoint receptors PD-1, LAG-3 and TIM-3 within the whole tumor post ICT01 treatment (p=0.013). Specific analysis of stroma and tumor areas within the biopsies confirmed the increased density of PD1+LAG3+TIM3+CD8 T cells in the tumors collected after ICT01 treatment in both patients that received ICT01 monotherapy (group A) and ICT01+pembrolizumab (group C) (FIG. 3).

These findings emphasize the rationale for combining ICT01 with other immune checkpoint therapies beyond anti-PD-1 to potentiate effector immune cell responses.

### 3.4 LAG-3 Modulates ICT01-Mediated Vγ9Vδ2 T Cell Activation and Expansion in HD Hu-PBMC

The involvement of LAG-3, TIM-3 and TIGIT on activation and expansion of Vγ9Vδ2 T cells that were sub-optimally activated with ICT01 was evaluated by incubating HD hu-PBMC with low ICT01 dose in presence of specific blocking mAbs for LAG-3, TIM-3 or TIGIT or their corresponding isotypes (mIgG1). As expected, ICT01 induced Vγ9Vδ2 T cell activation and expansion as shown by an increase of CD25 Mean Fluorescence Intensity (MFI) at day 3 and absolute count of Vγ9Vδ2 T cells at day 6 respectively. Interestingly, only LAG-3 blockade improved ICT01-mediated Vγ9Vδ2 T cell activation and expansion in vitro as compared to control isotype whereas TIM-3 and TIGIT blockade had minor impact (FIG. 4A). Additionally, LAG-3 blockade also enhanced the IFN-γ and granzyme B secretion in supernatant of ICT01-treated HD Hu-PBMC (FIG. 4B), suggesting a crucial role of LAG-3 in regulating the ability of ICT01-mediated Vγ9Vδ2 T cells to produce IFN-γ and cytolytic granules.

### 3.5 LAG-3, TIM-3 and TIGIT Modulate ICT01-Mediated Killing of Ovarian Cancer Cell Line (SKOV3) by Vγ9Vδ2 T Cells.

Next, the ability of anti-TIM-3, anti-LAG-3 and anti-TIGIT blocking mAbs to modulate tumor killing by ICT01-activated Vγ9Vδ2 T cells was tested. For this purpose, in vitro expanded γδ T cells that expressed TIM-3, LAG-3 and TIGIT at plasma membrane (as shown in FIG. 5A) were cocultured with the SKOV3 cell line. ICT01 treatment triggered the tumor killing ability of expanded Vγ9δ2 T cells as shown by a decrease of the live tumor cell percentages at the end of the co-culture (gated as negative for live/dead and annexin V staining) determined by flow cytometry (FIG. 5B). This killing ability of ICT01-activated Vγ9Vδ2 T cells was significantly enhanced by anti-TIM-3, anti-LAG-3 and anti-TIGIT blocking mAbs demonstrating the role of these immune checkpoint receptors to negatively modulate Vγ9Vδ2 T cell function (FIG. 5B).

### 3.6 LAG-3, TIM-3 and TIGIT Modulate ICT01-Mediated Killing of Leukemic Cell Line by Vγ9Vδ2 T Cells

Given the effect of blocking LAG-3, TIM-3, or TIGIT on the ICT01-mediated killing of SKOV3 cells by Vγ9Vδ2 T cells, we explored the influence of anti-TIM-3, anti-LAG-3, and anti-TIGIT blocking mAbs on the cytotoxicity of ICT01-activated Vγ9Vδ2 T cells against the leukemia cell line THP-1. In vitro expanded Vγ9Vδ2 T cells expressing TIM-3, LAG-3, and TIGIT on their plasma membrane (FIG. 6A upper panel) were co-cultured with the leukemic cell line THP1, which expresses BTN3A and ligands for LAG-3, TIM-3, and TIGIT (FIG. 6A lower panel).

The use of ICT01 during the co-culture enhanced the tumor-killing capacity of expanded Vγ9Vδ2 T cells, as evidenced by a reduction in the percentage of viable tumor cells at the end of the co-culture (live cells are gated as negative for live/dead and annexin V staining), as determined by flow cytometry. Furthermore, the tumor-killing ability of ICT01-activated Vγ9Vδ2 T cells was significantly augmented by anti-TIM-3, anti-LAG-3, and anti-TIGIT blocking mAbs (FIG. 6B). These results confirmed the ones obtained using solid tumor cell lines and underscore the involvement of these immune checkpoint receptors in negatively modulating the function of Vγ9Vδ2 T cells.

### 3.7 ICT01 Plus LAG-3- or TIM-3-Blockade Combinations Improve Tumor Cell Killing by HD Hu-PBMC Over Single Agents

Subsequently, we examined the impact of the combination of ICT01 with immune checkpoint blockade on HD Hu-PBMC-mediated cytolysis of tumor target cells. This was achieved by co-culturing the ovarian cancer cell line SKOV3 with HD Hu-PBMC in the presence of therapeutic mAbs used as single agents or in combination. The growth of cancer cells over time was monitored by live imaging using an Incucyte instrument.

Treatment with ICT01 alone induced significant tumor cell killing by HD Hu-PBMC, as evidenced by a marked decrease in the count of target cells over time compared to the untreated condition (FIG. 7). Additionally, the humanized anti-LAG-3 (Relatlimab) and anti-TIM-3 (Sabatolimab) blocking antibodies demonstrated a reduction in target cell count compared to their respective isotypes (hIgG4) (FIGS. 7A & B respectively). Importantly, both the combination of ICT01 with anti-LAG-3 (FIG. 7A) or the combination of ICT01 with anti-TIM-3 (FIG. 7B) exhibited a significantly superior effect on HD Hu-PBMC-mediated killing of tumor cells compared to single agents.

### 3.8 the Recombinant LAG-3 Fc Fusion Protein Improves ICT01-Mediated Activation/Expansion of Vγ9Vδ2 T Cells in HD Hu-PBMC

In addition to the use of anti-LAG-3 blocking mAb, we evaluated the effect of soluble LAG-3 immunoglobulin fusion protein (LAG-3 Fc), an MHC class II agonist currently undergoing clinical trials, on the modulation of Vγ9Vδ2 T cell responses.

First, we investigated the ability of LAG-3 Fc to influence ICT01-mediated activation and expansion of Vγ9Vδ2 T cells. HD Hu-PBMC were incubated with a suboptimal concentration of ICT01 (0.1 μg/mL), with or without LAG-3 Fc or the corresponding control (Ig Fc), for 3 or 6 days. ICT01 stimulation induced Vγ9Vδ2 T cell activation and expansion, as evidenced by increased CD25 expression and absolute cell count, respectively (FIG. 8A). Importantly, LAG-3 Fc significantly potentiated these effects in most tested donors, as demonstrated by enhanced CD25 expression and absolute cell count (FIG. 8A). Additionally, the secretion of IFN-γ and granzyme B, measured by ELISA in the 3-day culture supernatant, increased upon treatment with LAG-3 Fc compared to the corresponding isotype (data not shown).

Next, we explored the synergistic potential of the combination of ICT01 and LAG-3 Fc on Vγ9Vδ2 T cell activation. HD Hu-PBMC were incubated with increasing concentrations of LAG-3 Fc or the corresponding control, with or without ICT01 used at suboptimal concentrations (0.03 and 0.1 μg/mL) for 2 days. LAG-3 Fc alone had a modest effect on resting Vγ9Vδ2 T cells, inducing a 1.3-fold increase in surface CD25 expression after 2 days of stimulation at 10 μg/mL. ICT01 used at the suboptimal concentrations of 0.03 and 0.1 μg/mL induced respectively 1.9- and 3.9-fold increases in surface CD25 expression on Vγ9Vδ2 T cells. Importantly, when ICT01 used at the same suboptimal concentrations was used together with 10 μg/mL of LAG-3 Fc, we observed a 9.3- and 31.5-fold increase in CD25 expression on Vγ9Vδ2 T cells demonstrating the synergistic activity of the two compounds (FIG. 8B). Additionally, IFN-γ and TNF-α secretion in the culture supernatant increased upon treatment with LAG-3 Fc compared to the corresponding control and was further enhanced in the presence of ICT01 (FIG. 8C). Furthermore, consistent with its established biological activity described in the literature, LAG-3 Fc induced a dose-dependent activation of NK cells and CD8+ T cells in HD Hu-PBMC. These effects were not changed with ICT01 treatment, in line with its specific activity on Vγ9Vδ2 T cells (FIG. 8D).

In summary, our findings demonstrate that LAG-3 Fc induces activation of effector immune cells and can synergistically act with ICT01 to trigger activation of Vγ9Vδ2 T cells in HD Hu-PBMC.

### 3.9. LAG-3 Fc Enhances ICT01-Mediated Cytotoxic Activity of Vγ9Vδ2 T Cells

Given the synergistic enhancement of Vγ9Vδ2 T cell activation by the combination of ICT01 and LAG-3 Fc, we examined the impact of this combination on the cytolysis of tumor cells. Ovarian cancer cell line SKOV3 and leukemia cell line THP1 were co-cultured with in vitro expanded Vγ9Vδ2 T cells.

First, we characterized the expression of MHC class II, the primary target of LAG-3 Fc, on both tumor cells and expanded Vγ9Vδ2 T cells. FIG. 9A illustrates that MHC class II was prominently expressed on the plasma membrane of the leukemia cell line THP1 but not on the ovarian cancer cell line SKOV3. High levels of MHC class II were also consistently detected on in vitro expanded Vγ9Vδ2 T cells from all tested donors (FIG. 9A).

Subsequently, we investigated the ability of the ICT01 plus LAG-3 Fc combination to modulate the cytotoxicity of in vitro expanded Vγ9Vδ2 T cells against THP1 (FIG. 9B) and SKOV3 (FIG. 9C) by monitoring Vγ9Vδ2 T cell degranulation using flow cytometry. ICT01 and LAG-3 Fc, when used individually, induced Vγ9Vδ2 T cell degranulation, as evidenced by an increase in CD107 expression on Vγ9Vδ2 T cells (FIGS. 9B and C). This effect was significantly enhanced when both agents were combined both when using THP1 (FIG. 9B) and SKOV3 (FIG. 9C) targets.

Altogether, our results unveil the remarkable synergy achieved by combining ICT01 with LAG-3 Fc to trigger a strong activation of Vγ9Vδ2 T cells and a robust cytolytic activity against cancer cells.

### 3.10. LAG-3 Fc can Directly Act on Vγ9Vδ2 T Cells

Given the significant MHC class II expression on expanded Vγ9Vδ2 T cells and the ability of LAG-3 Fc to induce Vγ9Vδ2 T cell degranulation even in the absence of MHC class II on target cells such as SKOV3, we hypothesized that LAG-3 Fc may have a direct activity on Vγ9δ2 T cells.

To address this question, we conducted a series of experiments, starting with the evaluation of LAG-3 Fc's activity on MHC class II high Vγ9δ2 T cells. Since MHC class II is strongly induced by ICT01 treatment, HD Hu-PBMC were pretreated with ICT01 for two days, prompting Vγ9δ2 T cells activation as shown by increase expression of the CD25 marker, and high MHC class II expression on the surface of Vγ9δ2 T cells (FIG. 10A). Subsequently, total CD3+ T cells (that include the Vγ9δ2 T cell subpopulation) were sorted from either untreated or ICT01-pretreated PBMC to eliminate all antigen-presenting, MHC class II-positive cells. Sorted T cells were then incubated with LAG-3 Fc or Ig Fc control for one day. FIG. 10B shows that while LAG-3 Fc had no effect on MHC class II low, non-preactivated cells, it remarkably triggers activation of MHC class II high, ICT01-preactivated Vγ9δ2 T cells, suggesting a direct effect of LAG-3 Fc on Vγ9δ2 T cell activation (FIG. 10B).

We confirmed these results using in vitro expanded Vγ9δ2 T cells known to express high surface MHC class II (FIG. 10C, left panel). In vitro expanded Vγ9δ2 T cells were cultured in presence of LAG-3 Fc or an irrelevant Ig Fc in absence of any additional cells, and degranulation was assessed by flow cytometry. In these conditions, LAG-3 Fc induces Vγ9δ2 T cell degranulation as depicted by increase CD107 compared to Ig Fc condition (FIG. 10C, right panel).

Altogether, these findings strongly suggest a direct effect of LAG-3 Fc on MHC class II positive Vγ9δ2 T cells.

1. A method for treating cancer in a subject in need thereof, said method comprising administering a therapeutically effective amount of a BTN3A activating antibody to said subject, in combination with a therapeutically effective amount of an immune checkpoint inhibitor, wherein said BTN3A activating antibody, either:
comprises (a) a variable heavy chain (VH) polypeptide comprising an amino acid sequence that is at least about 95%, 96%, 97%, 98%, 99% or 100% identical to the SEQ ID NO: 1, and (b) a variable light chain (VL) polypeptide comprising an amino acid sequence that is at least about 95%, 96%, 97%, 98%, 99% or 100% identical to SEQ ID NO: 2 or SEQ ID NO: 3;
comprises HCDRs1-3 of SEQ ID NO: 12-14 and LCDRs1-3 of SEQ ID NO:15-17;
comprises HCDRs1-3 of SEQ ID NO: 18-20 and LCDRs1-3 of SEQ ID NO: 21-23; or,
competes for binding with an antibody having a heavy chain of SEQ ID NO:4 and a light chain of SEQ ID NO:6,
and wherein said immune checkpoint inhibitor is selected from the group consisting of LAG-3 inhibitors, TIGIT inhibitors and TIM-3 inhibitors, and, optionally, further in combination with PD-1 inhibitors.

2. The method of claim 1, wherein said BTN3A activating antibody induces the activation of γδ T cells.

3. (canceled)

4. The method of claim 1, wherein said BTN3A activating antibody comprises HCDRs1-3 of SEQ ID NO: 12-14 and LCDRs1-3 of SEQ ID NO: 15-17.

5. The method of claim 1, wherein said BTN3A activating antibody is an antibody comprising or essentially consisting of a heavy chain of SEQ ID NO: 4 and a light chain of SEQ ID NO: 6.

6. The method of claim 1, wherein said BTN3A activating antibody comprises an IgG1 constant region with the triple mutations L247F L248E and P350S.

7. The method of claim 1, wherein said immune checkpoint inhibitor is a LAG-3 inhibitor, or a fixed dose combination comprising an anti-LAG-3 antibody which: either,
comprises (a) a variable heavy chain (VH) polypeptide comprising an amino acid sequence that is at least about 95%, 96%, 97%, 98%, 99% or 100% identical to the SEQ ID NO: 37, and (b) a variable light chain (VL) polypeptide comprising an amino acid sequence that is at least about 95%, 96%, 97%, 98%, 99% or 100% identical to SEQ ID NO: 38;
comprises HCDRs1-3 of SEQ ID NO:45-47 and LCDRs1-3 of SEQ ID NO:48, 43 and 49 or,
competes for binding with an anti-LAG-3 antibody selected from relatlimab and favezelimab.

8. The method of claim 1, wherein said immune checkpoint inhibitor is an anti-LAG-3 antibody administered at a dosage ranging from 0.1 to 10 mg/kg.

9. The method of claim 1, wherein said cancer is selected from the group consisting of melanoma (e.g. metastatic malignant melanoma), lung cancer (e.g. non-small cell lung cancer), breast cancer, prostate cancer, head and neck cancer (e.g. squamous cell carcinoma of head and neck), renal cancer, colorectal cancer, gastric cancer, esophageal cancer, liver cancer, pancreatic cancer, glioblastoma, ovarian cancer, cervical cancer, cholangiocarcinoma, bladder cancer, cancer of the kidney or ureter, sarcoma of soft tissue, and hematological malignancies including for example, acute myeloid leukemia, Hodgkin's lymphoma, multiple myeloma, diffuse large B-cell lymphoma (DLBCL), and indolent non-Hodgkin lymphoma (NHL).

10. The method of claim 1, wherein said subject is refractory or relapsed after PD-1 inhibitor therapy.

11. The method of claim 1, wherein said BTN3A activating antibody is administered once every three weeks, or once every four weeks.

12. The method of claim 1, wherein the BTN3A activating antibody is administered intravenously, for example, at a unit dose of about 0.1 to about 200 mg.

13. The method of claim 1, wherein the BTN3A activating antibody is administered intravenously at a unit dose of about 0.1 to about 200 mg for 1 to 22 cycles.

14. The method of claim 1, wherein said immune checkpoint inhibitor is an anti-LAG-3/anti-PD-1 bispecific antibody which is administered intravenously at a unit dose of about 100 to about 1000 mg.

15. The method of claim 1, wherein said BTN3A antibody comprises a heavy chain of SEQ ID NO:4 and a light chain of SEQ ID NO:6, and said immune checkpoint inhibitor is nivolumab/relatlimab.

16. The method of claim 1, wherein said LAG3 inhibitor is a LAG-3 Fc fusion protein.

17. The method of claim 1, wherein said LAG3 inhibitor is a LAG-3 Fc fusion protein comprising the polypeptide of SEQ ID NO:30.

18. The method of claim 1, wherein said LAG3 inhibitor is eftilagimod alpha.

19. The method of claim 1, wherein said LAG3 inhibitor is selected from the group consisting of eftilagimod alpha, leramilimab, favezelimab, fianlimab, relatlimab, BI754111, TSR-033, INCAGN2385, Sym022, FS118, Tebotelimab, and XmAb22841.
